(19) United States
(12) Patent Application Publication
(10) Pub. No.: US 2025/0375512 A1
(43) Pub. Date: Dec. 11, 2025

(54) USE OF MATRIX BOUND VESICLES (MBV) AS VACCINE ADJUVANTS

(71) Applicant: University of Pittsburgh - Of the Commonwealth System of Higher Education, Pittsburgh, PA (US)

(72) Inventors: Stephen Francis Badylak, West Lafayette, IN (US); George S. Hussey, Cranberry Township, PA (US); Hector Capella, Pittsburgh, PA (US)

(73) Assignee: University of Pittsburgh - Of the Commonwealth System of Higher Education, Pittsburgh, PA (US)

(21) Appl. No.: 18/877,090

(22) PCT Filed: Jun. 22, 2023
(86) PCT No.: PCT/US2023/068905

§ 371 Date: Dec. 19, 2024

Related U.S. Application Data
(60) Provisional application No. 63/355,508, filed on Jun. 24, 2022.

(51) Int. Cl. A61K 39/09; A61K 39/00; A61K 39/12; A61K 39/39

(52) U.S. Cl. CPC A61K 39/092; A61K 39/0002; A61K 39/12; A61K 39/39; A61K 2039/521; A61K 2039/5252; A61K 2039/53; A61K 2039/55538; A61K 2039/55555; A61K 2039/55572

(57) ABSTRACT

Disclosed herein are extracellular matrix (ECM) compositions, specifically to compositions comprising matrix bound nanovesicles (MBV) and an immunogen, and the use of these compositions, e.g., in vaccination.

### CROSS REFERENCE TO RELATED APPLICATIONS

This claims the benefit of U.S. Provisional Patent Application No. 63/355,508, filed Jun. 24, 2022, which is hereby incorporated by reference in its entirety.

### FIELD OF THE DISCLOSURE

This disclosure is related to the field of extracellular matrix (ECM) compositions, specifically to compositions comprising matrix bound nanovesicles (MBV) and an immunogen, and the use of these compositions, e.g., in vaccination.

### REFERENCE TO AN ELECTRONIC SEQUENCE LISTING

The contents of the electronic sequence listing submitted herewith entitled sequence.xml having a size of 8,000 bytes, and a Date of Creation of Jun. 13, 2023, is herein incorporated by reference in its entirety.

### BACKGROUND

Vaccines generally contain at least two major components: an immunogen that serves as a target for facilitating an adaptive immune response and an adjuvant that enhances said immune response. Complete Freund's Adjuvant (CFA) is a suspension of dead mycobacteria in a liquid prepared from non-metabolizable oils. CFA is widely considered the “gold standard” to which all other adjuvants are compared because of its proven effectiveness for over 70 years in inducing adaptive immunity. However, CFA cannot be used as an adjuvant in human vaccination because of its toxic effects primarily related to its induction of unresolved granulomas and abscesses at the site of vaccination. Accordingly, there is a need for an adjuvant that is suitable for human vaccination that can also facilitate the induction of a robust immune response.

### SUMMARY OF THE DISCLOSURE

In some aspects, disclosed are method for inducing an immune response to a vaccine antigen in a subject. These methods include administering to the subject an effective amount of isolated mammalian extracellular matrix bound vesicles (MBV), and an effective amount of a vaccine comprising or encoding the vaccine antigen, wherein the MBV do not express CD63 and CD81 or are CD63^{lo}CD81^{lo}, thereby inducing the immune response to the vaccine antigen.

In more aspects, disclosed are pharmaceutical compositions including an effective amount of isolated mammalian extracellular matrix bound vesicles (MBV) that do not express CD63 and CD81 or are CD63loCD81lo, an effective amount of a vaccine comprising or encoding a vaccine antigen, and a pharmaceutically acceptable carrier. These pharmaceutical compositions are of use in the methods disclosed herein.

The foregoing and other features and advantages of the disclosure will become more apparent from the following detailed description of several aspects which proceeds with reference to the accompanying figures.

### BRIEF DESCRIPTION OF THE FIGURES

FIGS. 1A-1B. Comparison of surface markers for exosomes, bone microvesicles (MV) and MBV. The figure shows the results of EXO-CHECK™ Exosome Antibody Arrays (System Biosciences) comparing levels of the various markers noted in murine exosomes, murine bone matrix vesicles (bone MV), and murine matrix bound nanovesicles (MBV). FIG. 1A provides digital images of the arrays and FIG. 1B is a graph showing the relative expression of each of the noted markers in the exosomes versus bone MV versus MBV. The data show that MBV are different from exosomes, bone microvesicles (MV) based on the profile of surface markers. The MBV do not express or have low expression of CD63, EpCAM, ANXA5, TSG101, GM130, FLOT1, ICAM1, ALIX, and CD81, as compared to Bone MV or exosome levels of these markers as shown in the bar graphs in the lower panel.

FIG. 2. Western blot showing expression of annexin V and alkaline phosphatase Tissue Non-specific Alkaline Phosphatase (TNAP) in bone MV. The expression of the bone MV markers Annexin V, and Tissue Non-specific Alkaline Phosphatase (TNAP) were evaluated by western blot analysis. Lysate prepared from 1711A Cells was used as a positive control. The results of this experiment show that matrix bound nanovesicles (MBV) are devoid of any expression of both markers found in bone microvesicles, TNAP and Annexin V. Plasma exosomes do express Annexin V, but do not express TNAP. These results clearly distinguish MBV from both exosomes and bone microvesicles Notably, the MBV used were isolated from muscle tissue.

FIG. 3. Macrophage activation-gene expression showing the different effects of exosomes, MV and MBV. MBV have a differential immunomodulatory effect, namely they increase M2 macrophages, when compared to exosomes or bone MV which do not have this effect. Bone Marrow-Derived Macrophages (BMDM) harvested from mice were untreated (M0) or treated with the following test articles for 24 hours: IFNγ+LPS to induce an M1 phenotype (M1), IL-4 to induce an M2-like phenotype (M2), Exosomes derived from plasma, bone MV derived from 17A cells, or MBV isolated from muscle. After treatment, the fold change in the expression of the indicated genes was evaluated by qPCR. FIG. 3 shows the downregulation of the pro-inflammatory markers IL-6 and TNF-α by MBV are clearly distinguished from the downregulation of the same two inflammatory mediators by exosomes and bone MV. MBV had a potent anti-inflammatory effect; whereas exosomes and bone MV did not have this effect.

FIG. 4 is a schematic depiction of mouse immunization experiments performed in Example 1. Balb/c mice were injected with vaccine and administered MBV or methotrexate (MTX) on Day 0, with subsequent weekly administration of MBV or MTX for five weeks.

FIGS. 5A-5B are graphs showing levels of anti-pneumococcal polysaccharide titers of IgG (FIG. 5A) and IgM (FIG. 5B) measured in blood samples obtained from the Balb/c mice (n=3) 7 days after immunization with PNEUMOVAX® 23. ***, **, * indicate p<0.001, p<0.01, p<0.05 compared to sham (N=3).

FIGS. 6A-6B are graphs showing the levels of anti-pneumococcal polysaccharide titers of IgG (FIG. 6A) and IgM (FIG. 6B) measured in blood samples obtained from the Balb/c mice (n=3) 28 days after immunization with PNEUMOVAX® 23. ***, **, * indicate p<0.001, p<0.01, p<0.05 compared to sham (N=3).

FIG. 7 is a survival chart of Balb/c mice (n=6) challenged intraperitoneally with lethal, septic levels (1×10¹¹ CFU) of S. pneumoniae. Survival was quantified up to 14-days post-challenge. ** indicates p<0.01. All animals (other than sham) received the IL-12 adjuvant. Surprisingly, the animals that received the MBV had a statistically significant increase in survival compared to the sham, vaccine, and vaccine+MTX groups.

FIGS. 8A-8B are graphs showing the levels of anti-pneumococcal polysaccharide titers of IgG (FIG. 8A) and IgM (FIG. 8B) measure in blood samples obtained from the Balb/c mice (n=3) after intranasal S. pneumoniae challenge. ****, ***, **, * indicate p<0.0001, p<0.001, p<0.01, p<0.05. The animals that received the MBV produced antibodies in response to the vaccine. Accordingly, MBV did not interfere with the animals' ability to mount an immune response to the vaccine. All treated animals received IL-12.

FIGS. 9A-9D are bar graphs showing the levels of inflammatory cytokines TNF-α (FIG. 9A), IL-6 (FIG. 9B), IL-23 (FIG. 9C), and IFNγ (FIG. 9D) produced by macrophages after exposure to microbial antigens ex vivo. ****, ***, **, * indicate p<0.0001, p<0.001, p<0.01, p<0.05 (n=4). The macrophages were isolated from mice bone marrow 10 days after the mice were infected intranasally with S. pneumoniae. The results obtained using PPS show that memory for the specific antigen in the vaccine was not affected by the administration of MBV. The results obtained using LPS surprisingly show that macrophages from mice immunized with both the vaccine and MBV produced increased inflammatory response to other immunogens, indicating that the administration of MBV expanded the response of macrophages to other immunogens.

FIG. 10 is a series of graphs showing the change in RNA levels in vaccinated mice, vaccinated+MBV mice, and vaccinated+MTX mice as quantified by RT-qPCR of various inflammatory cytokines produced by macrophages isolated from day 10 post infection mice after exposure to microbial antigens ex vivo (LPS=lipopolysaccharide; PPS=pneumococcal polysaccharide). * indicates p<0.05 (n=4).

FIGS. 11A-11D are graphs showing the change in RNA levels in vaccinated mice, vaccinated+MTX mice, and vaccinated+MBV mice as quantified by RT-qPCR of RNA levels of genes for antigen presentation (CD74, MR1) and chemokines (CSF2, CxC12) produced by macrophages isolated from day 10 post infection mice after exposure to microbial antigens ex vivo (LPS=lipopolysaccharide; PPS=pneumococcal polysaccharide). * indicates p<0.05, ** indicates p<0.01, *** indicates p<0.001 (N=4). The data show that the macrophages' role in antigen presentation was not compromised by the administration of MBV.

FIGS. 12A-12E provide a histological analysis of lungs of mice at 10 days post-infection intranasally with S. pneumoniae. FIG. 12A shows micrographs of lung tissue subject to HE staining and picrosirius red staining (collagen deposition). In the 20XHE micrographs, the tissue from the Vaccine+MBV group showed less polarization. FIG. 12B provides brightfield and 5× polarized images of the lung tissue. FIG. 12 C is a graph quantifying cell surface area coverage (cells/mm²) in lung tissue samples and shows there was no different in total cells between the control and treated groups. FIG. 12D is a graph providing a machine learning quantitative analysis of cell clustering (how close together cells are) in lung tissue samples based on quantifying the number of cells (%). The data shows there were no differences in cellularity among groups. FIG. 12E is a graph quantifying the area (%) occupied by cells based on collagen staining, where % Red refers to the presence of mature collagen and % yellow and % green refer to the presence of immature collagen, and shows that administration of MBV reduced the level of fibrosis in lung tissue as compared to other groups as demonstrated by the statistically significant lower levels of mature collagen deposition (% red). Reduction in fibrosis prevents loss of lung function due to infection in the lungs.

FIG. 13A shows micrographs of immunolabeled myeloid cells (Cd11b+), T-Cells (CD4+) and cytotoxic cells (CD8+) in lung sections from mice 10 days post-infection. HE staining and picrosirius red staining (collagen deposition) were used. FIGS. 13B-13D are graphs quantifying the types of cells stained (CD11b+, CD4+, and CD8+) in the lung tissue samples. FIG. 13E is a graph showing the ratio of CD4+/CD8+ quantified in the stained tissue. FIG. 13B shows that MBV administration did not impact myeloid cell generation (e.g., macrophages) as the levels of CD11b+ cells in the vaccine and vaccine+MBV groups were not statistically different. FIG. 13C shows that there was a decrease in the number of CD4+ cells in the MBV treated group, indicating that MBV induced immune modulation (but not immunosuppression) following administration of the MBV. In FIG. 13D, the data show that administration of MBV does not impact the CD8+ T cell response induced by the vaccine. As shown in FIG. 13E, there was no difference in the CD4/CD8 ratio between the administration of the vaccine and the vaccine with the MBV, indicating that MBV did not interfere with the animals mounting an active immune response to the vaccine.

### DETAILED DESCRIPTION

MBV are an integral component of the ECM, are distinct from exosomes, and effectively redirect hyperinflammation in preclinical models (Hussey G S, et al. (2020) Sci Adv 6(12):eaay4361; van der Merwe Y, et al. (2019) Sci Rep 9(1):3482). MBV contain immunomodulatory miRNA, proteins, and lipids and are rapidly taken up by macrophages, triggering signaling cascades and modulating gene expression essential for phenotype switching.

Cytokine cargo stored within MBV support reparative and regulatory M2 macrophages and control bacterial infections and inflammation after acute lung injury (Liu Q, et al. (2019) JCI Insight 4(6)). Matrix Bound Nanovesicles (MBV) activate the M2-like reparative and anti-inflammatory macrophage phenotype. Studies have shown that MBV are a distinct class of extracellular vesicle separate from exosomes found in body fluids (Hussey G S, et al. (2020) Sci Advances. 6(12):eaay4361). As MBV survive even harsh tissue decellularization processes, they can play a fundamental role in tissue and organ development and homeostasis across species, as well as a regulatory role in the tissue response to injury. MBV may be derived from multiple, varied tissue sources.

Compared to exosomes present in body fluids, MBV are highly enriched in pro-resolving lipid mediators activated by different phospholipases dependent on the pro-/anti-inflammatory context of the extracellular environment (Hussey G S, et al. (2020) Sci Adv 6(12):eaay4361). Moreover, MBV are a rich and stable source of IL-33 that directs immune cells toward a reparative M2-like phenotype, while also stimulating repair and regulatory functions by TREG in the damaged lung (Liu Q, et al. (2019) JCI Insight 4(6)). IL-33 delivery reduces bacterial super-infections after H1N1 infections by improving bacterial clearance (Robinson K M, et al. (2018) Mucosal immunology. 11(1):199-208). Additionally, MBV are enriched in miRNA 125b-5p, 143-3p, and 145-5p. Inhibition of these miRNAs within macrophages is associated with a gene and protein expression profile more consistent with a proinflammatory rather than an anti-inflammatory/regulatory phenotype (Huleihel L, et al. (2017) Tissue Eng Part A, 23(21-22):1283-1294).

However, MBV have not previously been used with a vaccine. Disclosed herein are compositions that include MBV with a vaccine, as well as methods of vaccination that include administering MBV with a vaccine. As described herein, the systemic administration of MBV together with vaccination does not interfere with the humoral immune response and allows a robust antibody production against a pathogen. In addition, MBV administered systemically, e.g., by intramuscular injection, amplifies the immune response, triggering cross-immunity against other pathogens unspecific to the vaccination as demonstrated, for example, by higher IFNγ and IL-23 production from myeloid immune cells (e.g., macrophages). In some aspects, MBV systemic administration modulates macrophages phenotype, affecting their memory and response after exposure to pathogens, while maintaining the response to known pathogens. These responses result in a higher myeloid immune reaction and adaptative cellular response activation.

### Terms

Unless otherwise noted, technical terms are used according to conventional usage. Definitions of many common terms in molecular biology may be found in Krebs et al. (eds.), Lewin's genes XII, published by Jones & Bartlett Learning, 2017. The following explanations of terms and methods are provided to better describe the present disclosure and to guide those of ordinary skill in the art in the practice of the present disclosure.

The singular forms “a,” “an,” and “the” refer to one or more than one, unless the context clearly dictates otherwise. For example, the term “comprising a MBV” includes single or plural MBVs and is considered equivalent to the phrase “comprising at least one MBV.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.”

It is further to be understood that any and all molecular weight or molecular mass values, or proportions, given for compositions are approximate, and are provided for descriptive purposes, unless otherwise indicated. Dates of GENBANK® Accession Nos. referred to herein are the sequences available at least as early as Jun. 22, 2022. All references, patent applications and publications, and GENBANK® Accession numbers cited herein are incorporated by reference.

Unless otherwise indicated, “about” indicates within five percent. In case of conflict, the present specification, including explanations of terms, will control. In order to facilitate review of the various aspects of the disclosure, the following explanations of specific terms are provided:

Acid Protease: An enzyme that cleaves peptide bonds, wherein the enzyme has increased activity of cleaving peptide bonds in an acidic pH. For example and without limitation, acid proteases can include pepsin and trypsin.

Adjuvant: A component of an immunogenic composition used to enhance antigenicity. In some aspects, an adjuvant can include a suspension of minerals (alum, aluminum hydroxide, or phosphate) on which antigen is adsorbed; or water-in-oil emulsion, for example, in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). In some aspects, the adjuvant used in a disclosed immunogenic composition is a combination of lecithin and carbomer homopolymer (such as the ADJUPLEX™ adjuvant available from Advanced BioAdjuvants, LLC, see also Wegmann, Clin Vaccine Immunol, 22(9): 1004-1012, 2015). Additional adjuvants for use in the disclosed immunogenic compositions include the QS21 purified plant extract, Matrix M, AS01, MF59, and ALFQ adjuvants. Immunostimulatory oligonucleotides (such as those including a CpG motif) can also be used as adjuvants. Adjuvants include biological molecules (a “biological adjuvant”), such as costimulatory molecules. Exemplary adjuvants include IL-2, RANTES, GM-CSF, TNF-α, IFN-γ, G-CSF, LFA-3, CD72, B7-1, B7-2, OX-40L, 4-1BBL and toll-like receptor (TLR) agonists, such as TLR-9 agonists. The person of ordinary skill in the art is familiar with adjuvants (see, e.g., Singh (ed.) Vaccine Adjuvants and Delivery Systems. Wiley-Interscience, 2007).

Administration: The introduction of a composition (such as MBV or a pharmaceutical preparation that includes MBV) into a subject by a chosen route. The route can be local or systemic. For example, if the chosen route is intravenous, the composition is administered by introducing the composition into a vein of the subject. If the chosen route is local, the composition can be administered by introducing the composition directly into a tissue of the subject.

Animal: Living multi-cellular vertebrate organisms, a category that includes, for example, mammals and birds. The term “mammal” includes both human and non-human mammals. Similarly, the term “subject” includes both human and veterinary subjects.

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term “antigen” includes all related antigenic epitopes. “Epitope” or “antigenic determinant” refers to a site on an antigen, such as a polypeptide antigen, to which B and/or T cells respond. In one aspect, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, at least 5, at least 9, at least 10, at least 11, at least 12, or about 9-12 amino acids in a unique spatial conformation. Methods of determining spatial conformation of epitopes include, for example, x-ray crystallography and 2-dimensional nuclear magnetic resonance.

Biocompatible: Any material, that, when implanted in a mammalian subject, does not provoke an adverse response in the subject. A biocompatible material, when introduced into an individual, is able to perform its intended function, and is not toxic or injurious to that individual, nor does it induce immunological rejection of the material in the subject.

Carrier: An immunogenic molecule to which an antigen can be linked. When linked to a carrier, the antigen may become more immunogenic. Carriers are chosen to increase the immunogenicity of the antigen and/or to elicit antibodies against the carrier which are diagnostically, analytically, and/or therapeutically beneficial. Useful carriers include polymeric carriers, which can be natural (for example, proteins from bacteria or viruses), semi-synthetic or synthetic materials containing one or more functional groups to which a reactant moiety can be attached.

Centrifugation: The process whereby a centrifugal force is applied to a mixture, whereby more-dense components of the mixture migrate away from the axis of the centrifuge relative to other less-dense components in the mixture. The force that is applied to the mixture is a function of the speed of the centrifuge rotor, and the radius of the spin. In most applications, the force of the spin will result in a precipitate (a pellet) to gather at the bottom of the centrifuge tube, where the remaining solution is properly called a “supernate” or “supernatant.” In other similar applications, a density-based separation or “gradient centrifugation” technique is used to isolate a particular species from a mixture that contains components that are both more dense and less dense than the desired component.

During the circular motion of a centrifuge rotor, the force that is applied is the product of the radius and the angular velocity of the spin, where the force is traditionally expressed as an acceleration relative to “g,” the standard acceleration due to gravity at the Earth's surface. The centrifugal force that is applied is termed the “relative centrifugal force” (RCF), and is expressed in multiples of “g.”

Contacting: Placement in direct physical association, which can be in solid or liquid form.

Cytokine: The term “cytokine” is used as a generic name for a diverse group of soluble proteins and peptides that act as humoral regulators at nano- to picomolar concentrations and which, either under normal or pathological conditions, modulate the functional activities of individual cells and tissues. These proteins also mediate interactions between cells directly and regulate processes taking place in the extracellular environment. Examples of cytokines include, but are not limited to, tumor necrosis factor (TNF)-α, interleukin (IL)-6, IL-10, IL-12, IL-23, transforming growth factor, and interferon (IFN)-γ.

Detecting: To identify the existence, presence, or fact of something. General methods of detecting may be supplemented with the protocols and reagents disclosed herein. For example, included herein are methods of detecting the level of a protein in a sample or a subject.

Diagnosis: The process of identifying a disease by its signs, symptoms and results of various tests. The conclusion reached through that process is also called “a diagnosis.” Forms of diagnostic testing commonly performed include, without limitation, blood tests, medical imaging, and biopsy.

Effective amount: An amount of agent, such as an immunogen, that is sufficient to elicit a desired response, such as an immune response in a subject. It is understood that to obtain a protective immune response against an antigen of interest can require multiple administrations of a disclosed immunogen, and/or administration of a disclosed immunogen as the “prime” in a prime boost protocol wherein the boost immunogen can be different from the prime immunogen.

Accordingly, an effective amount of a disclosed immunogen can be the amount of the immunogen sufficient to elicit a priming immune response in a subject that can be subsequently boosted with the same or a different immunogen to elicit a protective immune response.

Enriched: A process whereby a component of interest, such as a nanovesicle, that is in a mixture has an increased ratio of the amount of that component to the amount of other undesired components in that mixture after the enriching process as compared to before the enriching process.

Extracellular matrix (ECM): A complex mixture of structural and functional biomolecules and/or biomacromolecules including, but not limited to, structural proteins, specialized proteins, proteoglycans, glycosaminoglycans, and growth factors that surround and support cells within tissues and, unless otherwise indicated, is acellular. ECM preparations can be considered to be “decellularized” or “acellular”, meaning the cells have been removed from the source tissue through processes described herein and known in the art. By “ECM-derived material,” such as an “ECM-derived nanovesicle,” “Matrix bound nanovesicle,” “MBV” or “nanovesicle derived from an ECM” it is meant a nanovesicle that is prepared from a natural ECM or from an in vitro source wherein the ECM is produced by cultured cells.

Exogenous: Originating from a different source.

Immunogenic conjugate: A composition composed of at least two heterologous molecules (such as an immunogen and a carrier, such as a protein carrier) linked together that stimulates or elicits an immune response to a molecule in the conjugate in a vertebrate. In some aspects where the conjugate include a viral antigen, the immune response is protective in that it enables the vertebrate animal to better resist infection from the virus from which the antigen is derived.

Immune response: A response of a cell of the immune system, such as a B cell, T cell, or monocyte, to a stimulus. In one aspect, the response is specific for a particular antigen (an “antigen-specific response”). In one aspect, an immune response is a T cell response, such as a CD4+ response or a CD8+ response. In another aspect, the response is a B cell response, and results in the production of specific antibodies. A “humoral immune response” refers to an immune response mediated by antibody molecules and is a response of the adaptive immune system. The adaptive immune system also includes “cell-mediated immune response” which refers to an adaptive immune response by T-cells to pathogens presented on the surface of cells. An “innate immune response” is not specific to a pathogen, but rather a general immune response mounted by the innate immune system which provides a first line of defense again common pathogens and involves various cells and proteins that trigger inflammation and ultimately activate the adaptive immune response. “Priming an immune response” refers to treatment of a subject with a “prime” immunogen to induce an immune response that is subsequently “boosted” with a boost immunogen. Together, the prime and boost immunizations produce the desired immune response in the subject. “Enhancing an immune response” refers to co-administration of an adjuvant, e.g., MBV, and an immunogenic agent, wherein the adjuvant increases the desired immune response to the immunogenic agent compared to administration of the immunogenic agent to the subject in the absence of the adjuvant. In some aspects, the immune response is a protective immune response. As used herein, “a protective immune response” is an immune response to protect the subject against a future infection, disease, or disorder. In some aspects, the immune response is a therapeutic immune response. As used herein, “a therapeutic immune response” is an immune response used as a therapy for a disease in a subject, e.g., cancer or a virus.

Immunogen: A protein or a portion thereof that is capable of inducing an immune response in a mammal, such as a mammal infected or at risk of infection with a pathogen. “Vaccine antigen” and “immunogen” are used interchangeably in the disclosure.

Immunogenic composition: A composition comprising a disclosed immunogen, or a nucleic acid molecule or vector encoding a disclosed immunogen, that elicits a measurable cytotoxic T lymphocyte (CTL) response against the immunogen, or elicits a measurable B cell response (such as production of antibodies) against the immunogen, when administered to a subject. It further refers to isolated nucleic acids encoding an immunogen, such as a nucleic acid that can be used to express the immunogen (and thus be used to elicit an immune response against this immunogen). For in vivo use, the immunogenic composition will typically include the protein or nucleic acid molecule in a pharmaceutically acceptable carrier and may also include other agents, such as an adjuvant.

Inhibiting or treating a disease: Inhibiting the full development of a disease or condition. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. Inhibiting a disease can include preventing or reducing the risk of the disease, such as preventing or reducing the risk of infection. The beneficial effect can be evidenced, for example, by a delayed onset of clinical symptoms of the disease in a susceptible subject, a reduction in severity of some or all clinical symptoms of the disease, a slower progression of the disease, a reduction in the viral load, an improvement in the overall health or well-being of the subject, or by other parameters that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid, protein cell, or nanovesicle) has been substantially separated or purified away from other biological components in the cell of the organism or the ECM, in which the component naturally occurs. Nucleic acids and proteins that have been “isolated” include nucleic acids and proteins purified by standard purification methods. MBV that have been isolated are removed from the fibrous materials of the ECM. The term also embraces nucleic acids and proteins prepared by recombinant expression in a host cell as well as chemically synthesized nucleic acids.

Isotonic Buffered Solution: A solution that is buffered to a pH between 7.2 and 7.8 and that has a balanced concentration of salts to promote an isotonic environment.

Lysyl oxidase (Lox): A copper-dependent enzyme that catalyzes formation of aldehydes from lysine residues in collagen and elastin precursors. These aldehydes are highly reactive, and undergo spontaneous chemical reactions with other lysyl oxidase-derived aldehyde residues, or with unmodified lysine residues. In vivo, this results in cross-linking of collagen and elastin, which plays a role in stabilization of collagen fibrils and for the integrity and elasticity of mature elastin. Complex cross-links are formed in collagen (pyridinolines derived from three lysine residues) and in elastin (desmosines derived from four lysine residues) that differ in structure. The genes encoding Lox enzymes have been cloned from a variety of organisms (Hamalainen et al., Genomics 11:508, 1991; Trackman et al., Biochemistry 29:4863, 1990; incorporated herein by reference). Residues 153-417 and residues 201-417 of the sequence of human lysyl oxidase have been shown to be important for catalytic function. There are four Lox-like isoforms, called LoxL1, LoxL2, LoxL3 and LoxL4.

Macrophage: A type of white blood cell that phagocytoses and degrades cellular debris, foreign substances, microbes, and cancer cells. In addition to their role in phagocytosis, these cells play an important role in development, tissue maintenance and repair, and in both innate and adaptive immunity in that they recruit and influence other cells including immune cells such as lymphocytes. Macrophages can exist in many phenotypes, including phenotypes that have been referred to as M1 and M2. Macrophages that perform primarily pro-inflammatory functions are called M1 macrophages (CD86⁺/CD68⁺), whereas macrophages that decrease inflammation and encourage and regulate tissue repair are called M2 macrophages (CD206⁺/CD68⁺). The markers that identify the various phenotypes of macrophages vary among species. It should be noted that macrophage phenotype is represented by a spectrum that ranges between the extremes of M1 and M2. F4/80 (encoded by the adhesion G protein coupled receptor E1 (ADGRE1) gene) is a macrophage marker, see GENBANK® Accession No. NP_001243181.1, Apr. 6, 2018, and NP_001965, Mar. 5, 2018, both incorporated herein by reference. Without wishing to be bound by theory, it is believed that MBV have the ability to modulate the phenotype of macrophages, leading to an increase in M2-like, regulatory, or pro-remodeling macrophages. The effect of MBV on macrophages is further characterized in WO 2017/151862A1, incorporated herein by reference in its entirety. In some aspects, MBV of the present invention can be used to induce an M2 phenotype in macrophages and inhibit M1 macrophages in a subject.

MicroRNA: A small non-coding RNA that is about 17 to about 25 nucleotide bases in length, that post-transcriptionally regulates gene expression by typically repressing target mRNA translation. A microRNA (“miRNA” or “miR”) can function as negative regulators, such that greater amounts of a specific miRNA will correlates with lower levels of target gene expression. There are three forms of miRNAs, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nucleotide overhang at the 3′ end. The cleavage product, the premature miRNA (pre-miRNA) is about 60 to about 110 nucleotides long with a hairpin structure formed in a fold-back manner. Pre-miRNA is transported from the nucleus to the cytoplasm by Ran-GTP and Exportin-5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nucleotides in length.

Nanovesicle: An extracellular vesicle that is a nanoparticle of about 10 to about 1,000 nm in diameter. Nanovesicles are lipid membrane bound particles that carry biologically active signaling molecules (e.g. microRNAs, proteins) among other molecules. Generally, the nanovesicle is limited by a lipid bilayer, and the biological molecules are enclosed and/or can be embedded in the bilayer. Thus, a nanovesicle includes a lumen surrounded by plasma membrane. The different types of vesicles can be distinguished based on diameter, subcellular origin, density, shape, sedimentation rate, lipid composition, protein markers, nucleic acid content and origin, such as from the extracellular matrix or secreted. A nanovesicle can be identified by its origin, such as a matrix bound nanovesicle from an ECM (see above), protein content and/or the miR content.

An “exosome” or “liquid phase extracellular vesicle (EV)” is a membranous vesicle which is secreted by a cell, and ranges in diameter from 10 to 150 nm. Generally, late endosomes or multivesicular bodies contain intralumenal vesicles which are formed by the inward budding and scission of vesicles from the limited endosomal membrane into these enclosed vesicles. These intralumenal vesicles are then released from the multivesicular body lumen into the extracellular environment, typically into a body fluid such as blood, cerebrospinal fluid or saliva, during exocytosis upon fusion with the plasma membrane. An exosome is created intracellularly when a segment of membrane invaginates and is endocytosed. The internalized segments which are broken into smaller vesicles and ultimately expelled from the cell contain proteins and RNA molecules such as mRNA and miRNA. Plasma-derived exosomes largely lack ribosomal RNA. Extra-cellular matrix derived exosomes include specific miRNA and protein components, and have been shown to be present in virtually every body fluid such as blood, urine, saliva, semen, and cerebrospinal fluid. Exosomes can express CD11c, CD63, CD81, and/or CD9, and thus can be CD11c⁺ and/or CD63⁺ and/or C81⁺ and/or CD9⁺. Exosomes do not have high levels of lysyl oxidase on their surface.

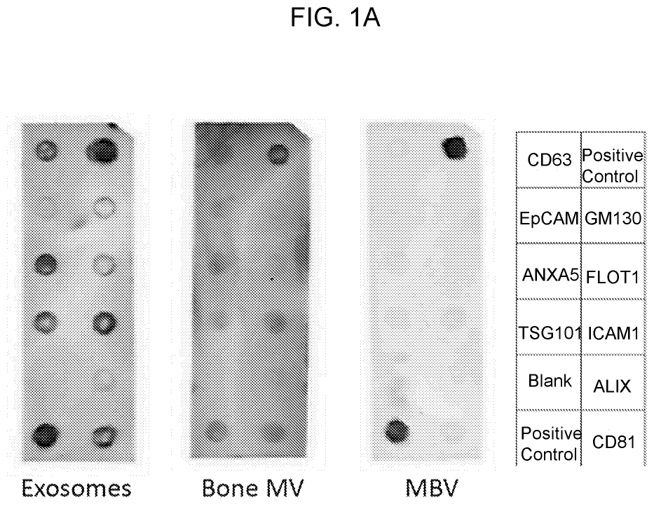

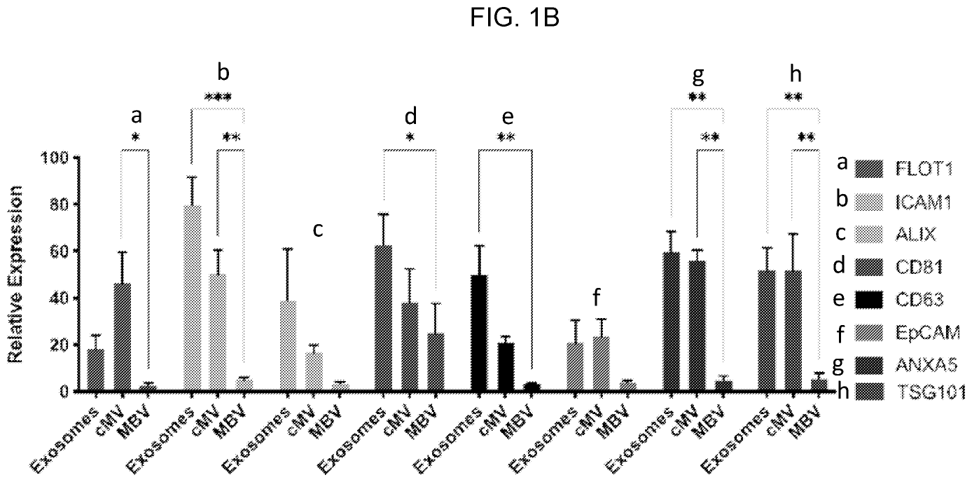

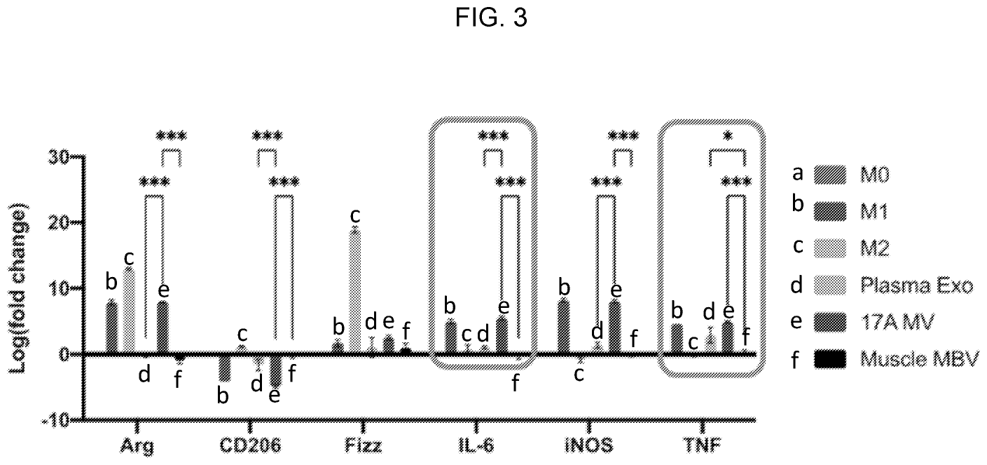

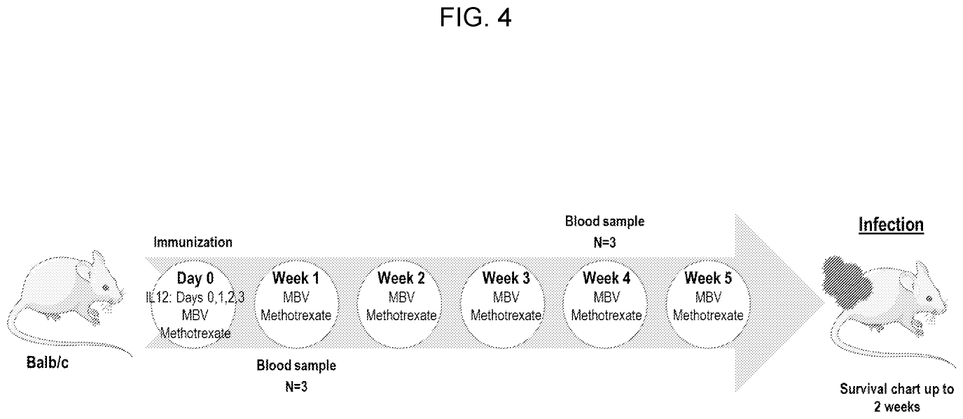

A “nanovesicle derived from an ECM,” “matrix bound nanovesicle,” “MBV” or an “ECM-derived nanovesicle” all refer to the same membrane bound particles, ranging in size from 10 nm-1000 nm, present in the extracellular matrix, which contain biologically active signaling molecules such as protein, lipids, nucleic acid, growth factors and cytokines that influence cell behavior. The terms are interchangeable and refer to the same vesicles. These nanovesicles are embedded within, and bound to, the ECM and are not simply attached to the surface or circulating freely in body fluids. These nanovesicles are resistant to harsh isolation conditions, such as freeze-thawing and digestion with proteases such as pepsin, elastase, hyaluronidase, proteinase K, and collagenase, and digestion with detergents. MBV are distinct from other extracellular vesicles including exosomes and have a phospholipid composition distinct from exosomes. MBV do not express alkaline phosphatase. In certain circumstances, MBV can also be distinguished from exosomes based on the absence of certain markers commonly attributed to exosomes. See for example, FIGS. 1-3. For example, MBV have been shown to not express or to express barely detectable levels of EpCAM, ANXA5 (Annexin V), TSG101, GM130, FLOT1, ICAM1, and/or ALIX1 compared to bone microvesicles and exosomes, thereby distinguishing them from exosomes and bone microvesicles. Accordingly, in some aspects, MBV do not express or have barely detectable levels of EpCAM, ANXA5, TSG101, GM130, FLOT1, ICAM1, and/or ALIX1.

In addition, MBV do not express tissue non-specification alkaline phosphatase, distinguishing them further from bone microvesicles.

In some aspects, MBV are characterized by expression of myeloperoxidase, while expressing little to none of the cytokines CRP, EGF, Osteoprogeterin, Pentraxin 2, RBP4 and Reg3G.

In some aspects, MBV are characterized by one or more of the following features of protein expression or lipid content:
(i) MBV do not express one or more of CD63 and/or CD81 and/or CD9 or have low or barely detectable levels of CD63 and/or CD81 and/or CD9 (CD63^{lo} and/or CD81^{lo} and/or CD9^{lo}) (see, e.g., FIG. 1) compared with other vesicles, such as exosomes. A variety of methods can be used to distinguish low, barely detectable, or absent expression of CD63 and/or CD81 and/or CD9 in MBV, for example, antibody-based methods, such as western blotting or flow cytometry (see, e.g., Bashashati and Brinkman, Adv Bioinformatics, 2009:584603). In some aspects, MBV expression of CD63 and/or CD81 and/or CD9 is considered low or barely detectable compared with other vesicles where the expression of CD63 and/or CD81 and/or CD9 in MBV is at least one standard deviation or at least two standard deviations below the mean expression of other vesicles, such as exosomes;
(ii) MBV have a phospholipid content wherein at least 55% of total phospholipids comprise phosphatidylcholine (PC) and phosphatidyl inositol (PI) in combination;
(iii) MBV have a phospholipid content wherein 10% or less of total phospholipids comprise sphingomyelin (SM);
(iv) MBV have a phospholipid content wherein 20% or less of total phospholipids comprise phosphatidylethanolamine (PE);
(v) MBV have a phospholipid content wherein 15% or greater of the total phospholipid content comprises phosphatidylinositol (PI) with the percent representing the percent of lipid concentration.

In some aspects, MBV are characterized by all of the following features:
(i) do not express one or more of CD63 and/or CD81 and/or CD9 or have low or barely detectable levels of CD63 and/or CD81 and/or CD9 (CD63^{lo} and/or CD81^{lo} and/or CD9^{lo}) (as further described above);
(ii) a phospholipid content wherein at least 55% of total phospholipids comprise phosphatidylcholine (PC) and phosphatidyl inositol (PI) in combination;
(iii) a phospholipid content wherein 10% or less of total phospholipids comprise sphingomyelin (SM);
(iv) a phospholipid content wherein 20% or less of total phospholipids comprise phosphatidylethanolamine (PE); and
(v) a phospholipid content wherein 15% or greater of the total phospholipid content is phosphatidylinositol (PI).

In some aspects, MBV are characterized by all of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some aspects, MBV are characterized by one or more of the following features:
(i) a phospholipid content wherein at least 55% of total phospholipids comprise phosphatidylcholine (PC) and phosphatidyl inositol (PI) in combination;
(ii) a phospholipid content wherein 10% or less of total phospholipids comprise sphingomyelin (SM);
(iii) a phospholipid content wherein 20% or less of total phospholipids comprise phosphatidylethanolamine (PE); and
(iv) a phospholipid content wherein 15% or greater of the total phospholipid content is phosphatidylinositol (PI).

In some aspects, MBV are characterize by one or more of the following features:
(i) do not contain detectable levels of alkaline phosphatase;
(ii) do not contain detectable levels of osteopontin;
(iii) do not contain detectable levels of osteoprogeterin;
(iv) do not contain detectable levels of complement C5; and/or
(v) do not contain detectable levels of c-reactive protein.

In some aspects, MBV carry miR-145 and/or miR-181 as cargo.

The ECM from which MBV are isolated can be an ECM from a tissue, can be produced from cells in culture, or can be purchased from a commercial source.

Pharmaceutically acceptable carriers: The pharmaceutically acceptable carriers useful in the claimed pharmaceutical preparations are conventional. Remington: The Science and Practice of Pharmacy, 22nd ed., London, UK: Pharmaceutical Press, 2013, describes compositions and formulations suitable for pharmaceutical delivery of the vaccines and MBV, as herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical preparations to be administered can contain minor amounts of non-toxic auxiliary substances, such as wetting or emulsifying agents, preservatives, and pH buffering agents and the like, for example sodium acetate or sorbitan monolaurate.

Pharmaceutical agent: A chemical compound or composition capable of inducing a desired therapeutic or prophylactic effect when properly administered to a subject or a cell.

Phospholipid: A class of lipids having a structure consisting of two hydrophobic fatty acid tails and a hydrophilic head consisting of a phosphate group. Major classes of phospholipids include phosphatidylcholine (PC), phosphatidylethanolamine (PE), phosphatidylinositol (PI), phosphatidylserine (PS), phosphatidylglycerol (PG), sphingomyelin (SM), cardiolipin (CL), phosphatidic acid (PA), and bis-monoacylglycerophosphate (BMP). Phospholipids can be measured in a variety of ways. For example, liquid chromatography-mass spectrometry (LC-MS) based global lipidomics and redox lipidomics can be used. In some aspects, specific phospholipid content is indicated as the percent concentration of the total phospholipids (such as total phospholipids in MBV), where the percent concentration is weight/weight (w/w).

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Polypeptide: Any chain of amino acids, regardless of length or post-translational modification (e.g., glycosylation or phosphorylation). “Polypeptide” applies to amino acid polymers including naturally occurring amino acid polymers and non-naturally occurring amino acid polymer as well as in which one or more amino acid residue is a non-natural amino acid, for example, an artificial chemical mimetic of a corresponding naturally occurring amino acid. A “residue” refers to an amino acid or amino acid mimetic incorporated in a polypeptide by an amide bond or amide bond mimetic. A polypeptide has an amino terminal (N-terminal) end and a carboxy terminal (C-terminal) end. “Polypeptide” is used interchangeably with peptide or protein, and is used herein to refer to a polymer of amino acid residues.

Prime-boost immunization: An immunotherapy including administration of multiple immunogens over a period of time to elicit the desired immune response.

Prophylactic: as used herein refers to a medication or a treatment designed and used to prevent a disease or disorder from occurring. As used herein, the terms “prophylactic” and “prevention” are used interchangeably.

Purified: The term “purified” does not require absolute purity; rather, it is intended as a relative term. Thus, for example, a purified nucleic acid molecule preparation is one in which the nucleic referred to is more pure than the nucleic in its natural environment within a cell. For example, a preparation of a nucleic acid is purified such that the nucleic acid represents at least 50% of the total protein content of the preparation. Similarly, a purified MBV preparation is one in which the exosome is more pure than in an environment including cells, wherein there are microvesicles and exosomes. A purified population of nucleic acids or MBV is greater than about 90%, 91%, 92%, 93%, 94%, 95%, 96%, 97%, 98%, 99% or 100% pure, or free other nucleic acids or cellular components, respectively.

Subject: Human and non-human animals, including all vertebrates, such as mammals and non-mammals, such as non-human primates, mice, rabbits, sheep, dogs, cats, horses, cows, chickens, amphibians, and reptiles. In many aspects of the described methods, the subject is a human. “Subject” is used interchangeably with the term “patient.” A subject may be an individual diagnosed with a high risk of developing a disease or disorder, for example, an infectious disease or disorder (e.g., an immunocompromised individual, a healthcare professional), someone who has been diagnosed with a disease or disorder, for example, an infectious disease or disorder, someone who previously suffered from a disease or disorder, for example, an infectious disease or disorder, or an individual evaluated for symptoms or indications of a disease or disorder, for example, an infectious disease or disorder.

Therapeutically effective amount: A quantity of a specific substance, such as an MBV and/or a vaccine, sufficient to achieve a desired effect in a subject being treated. When administered to a subject, a dosage will generally be used that will achieve target tissue concentrations (for example, in the lung) that has been shown to achieve a desired in vitro effect.

Total phospholipid content: “Total phospholipids” or “total phospholipid content”, as used herein, with respect to MBV, refers to the sum of all phospholipids present in a given quantity of isolated MBV, i.e., MBV isolated from the ECM. MBV can be isolated, for example, by enzymatic digestion of decellularized ECM and differential centrifugation. The total phospholipid content can be determined by methods such as LC-MS based global lipidomics and redox lipidomics. The total phospholipid content is measured by weight. A percentage of the total phospholipid content refers to a percent concentration on a weight/weight basis.

Tumor associated antigen: Any antigen including but not limited to a protein, glycoprotein, ganglioside, carbohydrate, or lipid that is associated with cancer. Such antigen can be expressed on malignant cells or in the tumor microenvironment such as on tumor-associated blood vessels, extracellular matrix, mesenchymal stroma, or immune infiltrate.

Vaccine: A pharmaceutical composition that elicits a prophylactic or therapeutic immune response in a subject. A vaccine may contain one or more antigens or a polynucleotide encoding the antigen (e.g., an mRNA). In some cases, the immune response is a protective immune response. Typically, a vaccine elicits an antigen-specific immune response to an antigen of a pathogen, for example a viral pathogen, or to a cellular constituent correlated with a pathological condition. A vaccine may include a polynucleotide (such as a nucleic acid encoding a disclosed antigen), a peptide or polypeptide (such as a disclosed antigen), a virus, a cell or one or more cellular constituents.

Vaccine antigen: An antigen present in (or encoded by a nucleotide in) a vaccine that elicits an immune response (e.g., generation of an antigen-specific humor al or cellular immune response) when administered to a subject.

### Aspects

In some aspects, disclosed are method for inducing an immune response to a vaccine antigen in a subject. These methods include administering to the subject an effective amount of isolated mammalian extracellular matrix bound vesicles (MBV), and an effective amount of a vaccine including or encoding the vaccine antigen, wherein the MBV do not express CD63 and CD81 or are CD63^{lo}CD81^{lo}, thereby inducing the immune response to the vaccine antigen. In specific non-limiting examples, the subject is a human. In other non-limiting examples, the subject is a veterinary subject. In further aspects, the method includes administering an effective amount of a cytokine, such as, but not limited to, IL-12.

In some aspects, the immune response is a therapeutic immune response. In further aspects, the immune response is a protective immune response. In more aspects, the immune response includes inducing myeloid cells. In other aspects, the immune response includes inducing production of IgM and/or IgG antibodies to the vaccine antigen. In some aspects, the immune response includes inducing production of IFNγ and/or IL-23. In further aspects, the immune response includes at T cell response.

In some aspects, the vaccine antigen is a tumor associated antigen, a viral antigen, a fungal antigen, a parasitic antigen, or a bacterial antigen. In some aspects, the mRNA encodes a viral protein, a bacterial protein, a fungal protein, a parasitic protein, or a tumor associated protein. In more aspects, the vaccine includes mRNA encoding the vaccine antigen

In further aspects, the vaccine includes a live attenuated virus, bacteria, fungus, parasite, or portion thereof. In other aspects, the vaccine includes an inactivated virus, bacteria, fungus, parasite, or portion thereof. In further aspects, the vaccine is a heat killed vaccine or a chemically inactivated vaccine.

In some aspects, the vaccine antigen is a tumor cell or portion thereof, or a tumor associated antigen. In other aspects, the vaccine antigen is a viral protein, a bacterial protein, a fungal protein, or a parasitic protein.

In some aspects, the vaccine induces an immune response against a virus, wherein the virus is an Avian herpesvirus, a Bovine herpesvirus, a Canine herpesvirus, an Equine herpesvirus, herpes simplex virus-1 (HSV-1), herpes simplex virus-2 (HSV-2), Feline viral rhinotracheitis virus, Marek's disease virus, an Ovine herpesviruses, a Porcine herpesvirus, Pseudorabies virus, an Avian paramyxovirus, Bovine respiratory syncytial virus, Human respiratory syncytial virus (RSV), Canine distemper virus, Canine parainfluenza virus, canine adenovirus, canine parvovirus, monkeypox virus, Bovine Parainfluenza virus 3, Ovine parainfluenza 3, human parainfluenza, Rinderpest virus, Border disease virus, Bovine viral diarrhea virus (BVDV), BVDV Type I, BVDV Type II, chikungunya virus, Classical swine fever virus, cytomegalovirus (CMV), Avian Leukosis virus, Bovine immunodeficiency virus, Bovine leukemia virus, Bovine tuberculosis, Ebola virus, Epstein Barr Virus (EBV), Equine infectious anemia virus, Feline immunodeficiency virus, Feline leukemia virus (FeLV), a coronavirus, monkeypox virus, Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, a paramyxovirus, Porcine epidemic diarrhea virus, Porcine hemagglutinating encephalomyelitis virus, polio virus, Porcine parvovirus, Porcine Circovirus (PCV) Type I, PCV Type II, Porcine Reproductive and Respiratory Syndrome (PRRS) Virus, hepatitis virus, Rubella virus, Transmissible gastroenteritis virus, Turkey coronavirus, Bovine ephemeral fever virus, Rabies virus, small pox (variola) virus, Rotavirus, variola virus, varicella zoster virus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, severe acute respiratory syndrome coronavirus 1 (SARS-COV-1), severe acute respiratory syndrome coronavirus 2 (SARS-COV-2), a coronavirus, zika virus, a paramyxovirus, polio virus, hepatitis A virus, hepatitis C virus, Rubella virus, human parvovirus, norovirus, mumps virus, molluscum contagiosum virus, Rubeola virus, enterovirus, coxsackievirus, a picornavirus, or a herpesvirus. In some aspects, the vaccine induces an immune response against a bacteria, wherein the bacteria is Acinetobacter baumanii, an Actinobacillus sp., Actinomycetes, an Actinomyces sp. (such as Actinomyces israelii and Actinomyces naeslundii), an Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, a Bacillus sp. (such as Bacillus anthracis, Bacillus cereus, Bacillus subtilis, Bacillus thuringiensis, and Bacillus stearothermophilus), a Bacteroides sp. (such as Bacteroides fragilis), a Bartonella sp. (such as Bartonella bacilliformis and Bartonella henselae, Bifidobacterium sp., a Bordetella sp. (such as Bordetella pertussis, Bordetella parapertussis, and Bordetella bronchiseptica), a Borrelia sp. (such as Borrelia recurrentis, and Borrelia burgdorferi), a Brucella sp. (such as Brucella abortus, Brucella canis, Brucella melintensis and Brucella suis), Burkholderia sp. (such as Burkholderia pseudomallei and Burkholderia cepacia), a Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, a Citrobacter sp. Coxiella burnetii, Corynebacterium sp. (such as, Corynebacterium diphtheriae, Corynebacterium jeikeum and Corynebacterium), a Clostridium sp. (such as Clostridium perfringens, Clostridium difficile, Clostridium botulinum and Clostridium tetani), Eikenella corrodens, an Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli), an Enterococcus sp. (such as Enterococcus faecalis and Enterococcus faecium), an Ehrlichia sp. (such as Ehrlichia chafeensia and Ehrlichia canis), Erysipelothrix rhusiopathiae, an Eubacterium sp., Francisella tularensis, Fusobacterium nucleatum, Gardnerella vaginalis, Gemella morbillorum, a Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, a Klebsiella sp. (such as Klebsiella pneumoniae, Klebsiella granulomatis and Klebsiella oxytoca), a Lactobacillus sp., Listeria monocytogenes, Leptospira interrogans, Legionella pneumophila, Leptospira interrogans, a Peptostreptococcus sp., Moraxella catarrhalis, a Morganella sp., a Mobiluncus sp., a Micrococcus sp., a Mycobacterium sp. (such as Mycobacterium leprae, Mycobacterium tuberculosis, Mycobacterium intracellulare, Mycobacterium avium, Mycobacterium bovis, and Mycobacterium marinum), a Mycoplasma sp. (such as Mycoplasma pneumoniae, Mycoplasma hominis, and Mycoplasma genitalium), a Nocardia sp. (such as Nocardia asteroides, Nocardia cyriacigeorgica and Nocardia brasiliensis), a Neisseria sp. (such as Neisseria gonorrhoeae and Neisseria meningitidis), Pasteurella multocida, Plesiomonas shigelloides, a Prevotella sp., a Porphyromonas sp., Prevotella melaninogenica, a Proteus sp. (such as Proteus vulgaris and Proteus mirabilis), a Providencia sp. (such as Providencia alcalifaciens, Providencia rettgeri and Providencia stuartii), Pseudomonas aeruginosa, Propionibacterium acnes, Rhodococcus equi, a Rickettsia sp. (such as Rickettsia rickettsii, Rickettsia akari and Rickettsia prowazekii, Orientia tsutsugamushi (formerly: Rickettsia tsutsugamushi) and Rickettsia typhi), a Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), a Serratia sp. (such as Serratia marcesans and Serratia liquifaciens), a Shigella sp. (such as Shigella dysenteriae, Shigella flexneri, Shigella boydii and Shigella sonnei), a Staphylococcus sp. (such as Staphylococcus aureus, Staphylococcus epidermidis, Staphylococcus hemolyticus, Staphylococcus saprophyticus), a Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14, Streptococcus pneumoniae, rifampicin-resistant serotype 18C Streptococcus pneumoniae, tetracycline-resistant serotype 19F Streptococcus pneumoniae, penicillin-resistant serotype 19F, Streptococcus pneumoniae, and trimethoprim-resistant serotype 23F, Streptococcus pneumoniae, chloramphenicol-resistant serotype 4, Streptococcus pneumoniae, spectinomycin-resistant serotype 6B, Streptococcus pneumoniae, streptomycin-resistant serotype 9V, Streptococcus pneumoniae, optochin-resistant serotype 14 Streptococcus pneumoniae, rifampicin-resistant serotype 18C, Streptococcus pneumoniae, penicillin-resistant serotype 19F, Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, a Treponema sp. (such as Treponema carateum, Treponema petenue, Treponema pallidum and Treponema endemicum, Tropheryma whippelii, Ureaplasma urealyticum, a Veillonella sp., a Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), a Yersinia sp. (such as Yersinia enterocolitica, Yersinia pestis, and Yersinia pseudotuberculosis) and Xanthomonas maltophilia. In a specific non-limiting example, the bacteria is a Streptococcus sp. For example, the bacteria is S. pneumoniae.

In some aspects the bacteria is a gram positive bacteria. In some aspects, the bacteria is a gram negative bacteria. For example, when the bacteria is a gram positive bacteria, the vaccine antigen is a gram positive bacterial antigen. For example, the vaccine antigen can be a lipoteichoic acid (LTA). For example, when the bacteria is a gram negative bacteria, the vaccine antigen is a gram negative bacterial antigen. For example, the vaccine antigen can be a lipopolysaccharide (LPS).

In some aspects, the vaccine induces an immune response against a fungus, wherein the fungus is Trichophyton rubrum, T. mentagrophytes, Epidermophyton floccosum, Microsporum canis, Pityrosporum orbiculare (Malassezia furfur), a Candida sp. (such as Candida albicans), an Aspergillus sp. (such as Aspergillus fumigatus, Aspergillus flavus and Aspergillus clavatus), a Cryptococcus sp. (such as Cryptococcus neoformans, Cryptococcus gattii, a Cryptococcus laurentii and Cryptococcus albidus), a Histoplasma sp. (such as Histoplasma capsulatum), a Pneumocystis sp. (such as Pneumocystis jirovecii), or a Stachybotrys (such as Stachybotrys chartarum).

In more aspects, the vaccine induces an immune response against a parasite, wherein the parasite is a Plasmodium (Plasmodium falciparum, P. vivax, P. malariae), a Schistosome, a Trypanosome, a filarial nematodes, trichomoniasis, sarcosporidiasis, Taenia (T. saginata, T. solium), Leishmania, Toxoplasma gondii, Trichinelosis (Trichinella spiralis) or Coccidiosis (Eimeria species).

In further aspects, the vaccine induces a therapeutic immune response against a tumor cell in the subject, and wherein the therapeutic response is a reduction in tumor volume, tumor metastasis, or tumor number.

In further aspects, if the subject is infected with a pathogen to which the vaccine induces a protective immune response, the subject experiences increased IFNγ and/or IL-23 production compared to if the subject had received the vaccine without MBV. In more aspects, the subject has an increased cellular response, such as a cytotoxic T cell response, compared to if the subject had received the vaccine without MBV.

In some aspects, the disclosed methods induce an immune response to a vaccine antigen and to an antigen that is not the vaccine antigen. For example, the immune response to the antigen that is not the vaccine antigen comprises inducing production of IFNγ and/or IL-23. The vaccine antigen may be a bacterial and antigen and the method can induce an immune response also to a different bacterial antigen. For example, the vaccine antigen may be an S. pneumoniae bacterial antigen and the method induces an immune response to both the S. pneumoniae bacterial antigen and to lipopolysaccharide of a gram negative bacteria. The vaccine antigen may be a viral antigen and the method induces an immune response to a different viral antigen, for example, of a different virus. The vaccine antigen may be a fungal antigen and the method induces an immune response to a different fungal antigen, even to a different fungus.

In more aspects, the subject experiences increased survival compared to if the subject had received the vaccine without MBV.

In some aspects, the matrix bound vesicles (a) contain miR-145 and miR-181; (b) do not include alkaline phosphatase; and/or (c) do not include or have barely detectable levels of EpCAM, ANXA5, TSG101, GM130, FLOT1, ICAM1, and/or ALIX1.

In additional aspects, the matrix bound vesicles include: (a) a phospholipid content including at least 55% phosphatidylcholine (PC) and phosphatidyl inositol (PI) in combination; (b) a phospholipid content including 10% or less sphingomyelin (SM); (c) a phospholipid content including 20% or less phosphatidylethanolamine (PE); and/or (d) a phospholipid content including 15% or greater phosphatidylinositol (PI).

In more aspects, a composition including the effective amount of the vaccine and the effective amount MBV is administered to the subject. In other aspects, the vaccine and the MBV are each administered separately to the subject. In further aspects, the MBV and the vaccine are administered to the subject within 1 minutes, 2 minutes, 3 minutes, 4 minutes or 5 minutes of each other. In yet other aspects, the vaccine and the MBV are administered to the subject on a first date and the subject subsequently receives, on one or more subsequent dates, a further administration of an effective amount of MBV without further administration of vaccine. In some aspects, the first date and the subsequent date are separated by 1 week, 2 weeks, 3 weeks, 4 weeks, 5 weeks, 6 weeks, 7 weeks, 8 weeks, 3 months, 4, months, 5 months, or 6 months. In more aspects, the subject receives MBV on subsequent dates 1 week, 2 weeks, 3 weeks, 4 weeks, 5 weeks, 6 weeks, 7 weeks, 8 weeks, 3 months, 4, months, 5 months, and/or 6 months after the first date.

In some aspects, the vaccine is administered by intramuscular injection. In more aspects, the MBV are administered by intramuscular injection. In further aspects, the MBV are administered in (a) an amount of 1×10⁶ to 1×10²⁰ MBV per kg of body weight per administration; (b) an amount of 1×10⁶ to 1×10¹² MBV per kg of body weight per administration; or (c) the MBV are administered in an amount of 1×10⁹ to 1×10¹⁴ MBV per kg of body weight per administration.

In aspects, the MBV are derived from a mammalian extracellular matrix of urinary bladder, small intestine, heart, dermis, liver, kidney, uterus, brain, blood vessel, lung, bone, muscle, pancreas, placenta, stomach, spleen, colon, adipose tissue, or esophagus. In some aspects, the MBV are derived from urinary bladder matrix (UBM), small intestinal submucosa (SIS), or urinary bladder submucosa (UBS). In further aspects the mammal is a pig, cow, or sheep.

In more aspects, disclosed are pharmaceutical compositions including an effective amount of isolated mammalian extracellular matrix bound vesicles (MBV) that do not express CD63 and CD81 or are CD63^{lo}CD81^{lo}, an effective amount of a vaccine including or encoding a vaccine antigen, and a pharmaceutically acceptable carrier. These pharmaceutical compositions are of use in the methods disclosed herein. In further aspects, the composition further includes a vaccine adjuvant.

In some aspects, the vaccine includes mRNA encoding the vaccine antigen. In more aspects, the mRNA encodes a viral protein, a bacterial protein, a fungal protein, a parasitic protein, or a tumor associated protein.

In aspects, the vaccine is a live attenuated virus, bacteria, fungus, parasite, or portion thereof. In more aspects, the vaccine is an inactivated virus, bacteria, fungus, parasite, or portion thereof. In additional aspects, the vaccine is a heat killed vaccine or chemically inactivated vaccine.

In some aspects, the vaccine antigen is a viral protein, a bacterial protein, a fungal protein, a parasitic protein, or a tumor associated protein.

In more aspects, the virus is, or the viral protein is from an Avian herpesvirus, a Bovine herpesvirus, a Canine herpesvirus, an Equine herpesvirus, herpes simplex virus-1 (HSV-1), herpes simplex virus-2 (HSV-2), Feline viral rhinotracheitis virus, Marek's disease virus, an Ovine herpesviruses, a Porcine herpesvirus, Pseudorabies virus, an Avian paramyxovirus, Bovine respiratory syncytial virus, Human respiratory syncytial virus (RSV), Canine distemper virus, Canine parainfluenza virus, canine adenovirus, canine parvovirus, monkeypox virus, Bovine Parainfluenza virus 3, Ovine parainfluenza 3, human parainfluenza, Rinderpest virus, Border disease virus, Bovine viral diarrhea virus (BVDV), BVDV Type I, BVDV Type II, chikungunya virus, Classical swine fever virus, cytomegalovirus (CMV), Avian Leukosis virus, Bovine immunodeficiency virus, Bovine leukemia virus, Bovine tuberculosis, Ebola virus, Epstein Barr Virus (EBV), Equine infectious anemia virus, Feline immunodeficiency virus, Feline leukemia virus (FeLV), a coronavirus, monkeypox virus, Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, a paramyxovirus, Porcine epidemic diarrhea virus, Porcine hemagglutinating encephalomyelitis virus, polio virus, Porcine parvovirus, Porcine Circovirus (PCV) Type I, PCV Type II, Porcine Reproductive and Respiratory Syndrome (PRRS) Virus, hepatitis virus, Rubella virus, Transmissible gastroenteritis virus, Turkey coronavirus, Bovine ephemeral fever virus, Rabies virus, small pox (variola) virus, Rotavirus, variola virus, varicella zoster virus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, severe acute respiratory syndrome coronavirus 1 (SARS-COV-1), severe acute respiratory syndrome coronavirus 2 (SARS-COV-2), a coronavirus, zika virus, a paramyxovirus, polio virus, hepatitis A virus, hepatitis C virus, Rubella virus, human parvovirus, norovirus, mumps virus, molluscum contagiosum virus, Rubeola virus, enterovirus, coxsackievirus, a picornavirus, or a herpesvirus.

In some aspects, the bacteria is, or the bacterial protein is from Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (such as Mycobacterium leprae, Mycobacterium tuberculosis, Mycobacterium intracellulare, Mycobacterium avium, Mycobacterium bovis, and Mycobacterium marinum), Mycoplasma sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) or Xanthomonas maltophilia.

In some aspects, the fungus is, or the fungal protein is from, Trichophyton rubrum, T. mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), or Stachybotrys (such as Stachybotrys chartarum).

In some aspects, the parasite is, or the parasitic protein is from, Malaria (Plasmodium falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) or Coccidiosis (Eimeria species).

In further aspects, the vaccine antigen is a tumor cell or portion thereof, or a tumor associated protein.

In aspects, the matrix bound vesicles in the composition (a) contain miR-145 and miR-181; (b) do not include alkaline phosphatase; and/or (c) do not include or have barely detectable levels of EpCAM, ANXA5, TSG101, GM130, FLOT1, ICAM1, and/or ALIX1.

In more aspects, the matrix bound vesicles in the composition include: (a) a phospholipid content including at least 55% phosphatidylcholine (PC) and phosphatidyl inositol (PI) in combination; (b) a phospholipid content including 10% or less sphingomyelin (SM); (c) a phospholipid content including 20% or less phosphatidylethanolamine (PE); and/or (d) a phospholipid content including 15% or greater phosphatidylinositol (PI).

### Overview

Vaccination relies on the ability of the immune system to recognize pathogens that have been identified in the past, using the memory of the immune system to build a stronger and more effective immune defense, resulting in milder symptoms or no symptoms, when the subject is infected with the pathogen, leading to higher subject survival. Where vaccination has been widely proven effective at controlling many diseases and has saved millions of lives, the rise of new and more resistant pathogen variants and co-infections limit vaccine efficacy.

Adjuvants have been used to amplify the vaccine effect. However, the use of matrix-bound nanovesicles (MBV) as a vaccine adjuvant to induce an adaptive immune response has not been considered until this disclosure. As described herein, the systemic administration of MBV together with vaccination does not interfere with the humoral immune response and allows a robust antibody production against a pathogen. In addition, MBV administered systemically, e.g., by intramuscular injection, amplifies the immune response, triggering cross-immunity against other pathogens unspecific to the vaccination as demonstrated, for example, by higher IFNγ and IL-23 production from myeloid immune cells (e.g., macrophages). In some aspects, MBV systemic administration modulates macrophages phenotype, affecting their memory and response after exposure to pathogens, while maintaining the response to known pathogens. These responses result in a higher myeloid immune reaction and adaptative cellular response activation. Therefore, it is contemplated herein that the use of MBV as vaccine adjuvant poses a method for amplifying the effect of current (and future) vaccines by amplifying the myeloid and cellular response.

In some aspects, the present disclosure relates to use of MBV in facilitating a humoral response, e.g., in a subject receiving a treatment for facilitating a humoral response. In some aspects, the present disclosure relates to use of MBV in facilitating a cellular response, e.g., in a subject receiving a treatment for facilitating a cellular response, such as a cytotoxic T cell response. In some aspects, MBV are administered with a vaccine, e.g., as an adjuvant. The MBV can be administered with an effective amount of a cytokine, such as IL-12.

As shown herein, when a subject receives MBV administration concomitantly with a vaccine administration, the subject experiences an enhanced immune response to the vaccine antigen, e.g., an enhanced humoral immune response, as compared to a subject that receives only the vaccine.

Administration of MBV subsequent to the initial vaccine/MBV administration can also further enhance the immune response to the vaccine antigen. Administration of MBV as a vaccine adjuvant leads to improved survival upon challenge with a pathogen to which the vaccine is directed. In a human subject, improved survival may include avoidance of death, avoidance of hospitalization, reduction in severity of disease symptoms or experiencing asymptomatic disease upon infection with the pathogen to which the vaccine is directed.

Matrix Bound Nanovesicles Derived from an Extracellular Matrix (ECM)

Nanovesicles derived from ECM (also called matrix bound nanovesicles, “MBV”) are generally described in PCT Publication No. WO 2017/151862, WO 2018/204848, and WO 2019/213482, incorporated herein by reference. It is disclosed that MBV are embedded in the extracellular matrix. These MBV can be isolated and are biologically active. MBV do not express CD63 and CD81 or are CD63^{lo}CD81^{lo} and do not contain alkaline phosphatase. These MBV can be used for therapeutic purposes. In some aspects, the MBV do not contain or express alkaline phosphatase, osteopontin, osteoprogeterin, complement C5, and/or c-reactive protein.

An extracellular matrix is a complex mixture of structural and functional biomolecules and/or biomacromolecules including, but not limited to, structural proteins, specialized proteins, proteoglycans, glycosaminoglycans, and growth factors that surround and support cells within mammalian tissues and, unless otherwise indicated, is acellular. Generally, the disclosed MBV are embedded in any type of extracellular matrix (ECM) and can be isolated from this location. Thus, MBV are not detachably present on the surface of the ECM, and are not exosomes (also known as extracellular vesicles or EV) or bone (calcifying) matrix vesicles.

Extracellular matrices are disclosed, for example and without limitation, in U.S. Pat. Nos. 4,902,508; 4,956,178; 5,281,422; 5,352,463; 5,372,821; 5,554,389; 5,573,784; 5,645,860; 5,771,969; 5,753,267; 5,762,966; 5,866,414; 6,099,567; 6,485,723; 6,576,265; 6,579,538; 6,696,270; 6,783,776; 6,793,939; 6,849,273; 6,852,339; 6,861,074; 6,887,495; 6,890,562; 6,890,563; 6,890,564; and 6,893,666; each of which is incorporated by reference in its entirety). However, an ECM can be produced from any tissue, or from any in vitro source wherein the ECM is produced by cultured cells and comprises one or more polymeric components (constituents) of native ECM. ECM preparations can be considered to be “decellularized” or “acellular”, meaning the cells have been removed from the source tissue or culture.

In some aspects, the ECM is isolated from a vertebrate animal, for example, from a mammalian vertebrate animal including, but not limited to, human, monkey, pig, cow, sheep, etc. The ECM may be derived from any organ or tissue, including without limitation, urinary bladder, intestine (such as small intestine or large intestine), heart, dermis, liver, kidney, uterus, brain, blood vessel, lung, bone, muscle, pancreas, placenta, stomach, spleen, colon, adipose tissue, or esophagus. In specific non-limiting examples, the extracellular matrix is isolated from esophageal tissue, urinary bladder (such as urinary bladder matrix or urinary bladder submucosa), small intestinal submucosa, dermis, umbilical cord, pericardium, cardiac tissue, or skeletal muscle. The ECM can comprise any portion or tissue obtained from an organ, including, for example and without limitation, submucosa, epithelial basement membrane, tunica propria, etc. In one non-limiting aspect, the ECM is isolated from urinary bladder. In some aspects, the ECM is from a human subject. In other aspects, the ECM is from a porcine subject. In some aspect, the ECM is not porcine ECM. In some aspects, the ECM is not porcine UBM.

The ECM may or may not include the basement membrane. In another non-limiting aspect, the ECM includes at least a portion of the basement membrane. The ECM material may or may not retain some of the cellular elements that comprised the original tissue such as capillary endothelial cells or fibrocytes. In some aspects, the ECM contains both a basement membrane surface and a non-basement membrane surface.

In some aspects, the ECM is harvested from porcine urinary bladders (also known as urinary bladder matrix or UBM). Briefly, the ECM is prepared by removing the urinary bladder tissue from a mammal, such as a pig, and trimming residual external connective tissues, including adipose tissue. All residual urine is removed by repeated washes with tap water. The tissue is delaminated by first soaking the tissue in a de-epithelializing solution, for example and without limitation, hypertonic saline (e.g., 1.0 N saline), for periods of time ranging from ten minutes to four hours. Exposure to hypertonic saline solution removes the epithelial cells from the underlying basement membrane. Optionally, a calcium chelating agent may be added to the saline solution. The tissue remaining after the initial delamination procedure includes the epithelial basement membrane and tissue layers abluminal to the epithelial basement membrane. The relatively fragile epithelial basement membrane is invariably damaged and removed by any mechanical abrasion on the luminal surface. This tissue is next subjected to further treatment to remove most of the abluminal tissues but maintain the epithelial basement membrane and the tunica propria. The outer serosal, adventitial, tunica muscularis mucosa, tunica submucosa and most of the muscularis mucosa are removed from the remaining deepithelialized tissue by mechanical abrasion or by a combination of enzymatic treatment (e.g., using trypsin or collagenase) followed by hydration, and abrasion. Mechanical removal of these tissues is accomplished by removal of mesenteric tissues with, for example and without limitation, Adson-Brown forceps and Metzenbaum scissors and wiping away the tunica muscularis and tunica submucosa using a longitudinal wiping motion with a scalpel handle or other rigid object wrapped in moistened gauze. Automated robotic procedures involving cutting blades, lasers and other methods of tissue separation are also contemplated. After these tissues are removed, the resulting ECM consists mainly of epithelial basement membrane and subjacent tunica propria.

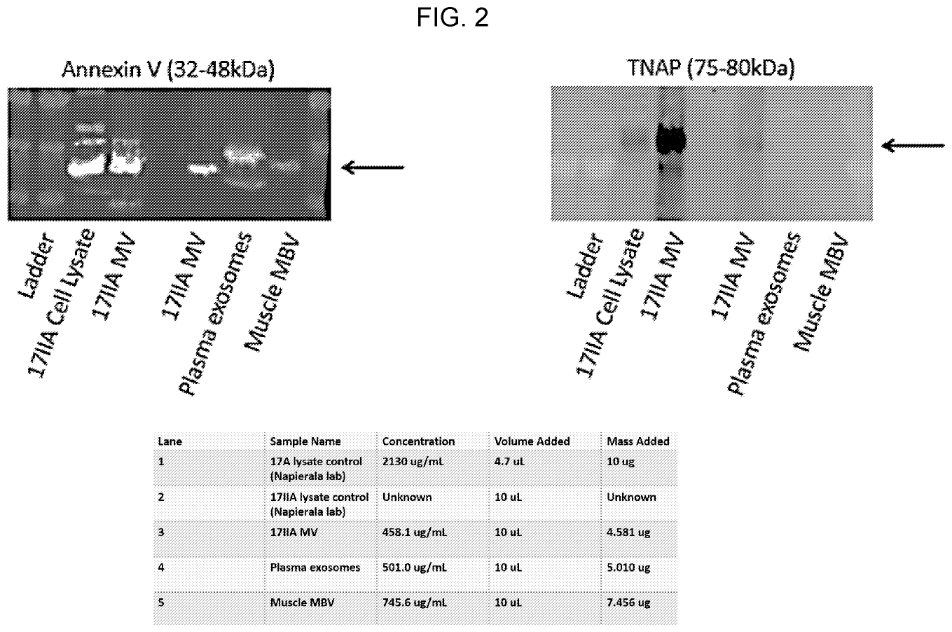

In another aspect, the ECM is prepared by abrading porcine bladder tissue to remove the outer layers including both the tunica serosa and the tunica muscularis using a longitudinal wiping motion with a scalpel handle and moistened gauze. Following eversion of the tissue segment, the luminal portion of the tunica mucosa is delaminated from the underlying tissue using the same wiping motion. Care is taken to prevent perforation of the submucosa. After these tissues are removed, the resulting ECM consists mainly of the tunica submucosa (see FIG. 2 of U.S. Pat. No. 9,277,999, which is incorporated herein by reference).

ECM can also be prepared as a powder. Such powder can be made according to the method of Gilbert et al., Biomaterials 26 (2005) 1431-1435, herein incorporated by reference in its entirety. For example, UBM sheets can be lyophilized and then chopped into small sheets for immersion in liquid nitrogen. The snap frozen material can then be comminuted so that particles are small enough to be placed in a rotary knife mill, where the ECM is powdered. Similarly, by precipitating NaCl within the ECM tissue the material will fracture into uniformly sized particles, which can be snap frozen, lyophilized, and powdered.

In one non-limiting aspect, the ECM is derived from small intestinal submucosa or SIS. Commercially available preparations include, but are not limited to, SURGISIS™, SURGISIS-ES™, STRATASIS™, and STRATASIS-ES™ (Cook Urological Inc.; Indianapolis, Ind.) and GRAFTPATCH™ (Organogenesis Inc.; Canton Mass.). In another non-limiting aspect, the ECM is derived from dermis. Commercially available preparations include, but are not limited to PELVICOL™ (sold as PERMACOL™ in Europe; Bard, Covington, Ga.), REPLIFORM™ (Microvasive; Boston, Mass.) and ALLODERM™ (LifeCell; Branchburg, N.J.). In another aspect, the ECM is derived from urinary bladder. Commercially available preparations include, but are not limited to UBM (ACell Corporation; Jessup, Md.).

MBV can be derived from (released from) an extracellular matrix using the methods disclosed below. In some aspects, the ECM is digested with an enzyme, such as pepsin, collagenase, elastase, hyaluronidase, or proteinase K, and the MBV are isolated. In other aspects, the MBV are released and separated from the ECM by changing the pH with solutions such as glycine HCL, citric acid, ammonium hydroxide, use of chelating agents such as, but not limited to, EDTA, EGTA, by ionic strength and or chaotropic effects with the use of salts such as, but not limited to potassium chloride (KCl), sodium chloride, magnesium chloride, sodium iodide, sodium thiocyanate, or by exposing ECM to denaturing conditions like guanidine HCl or Urea.

In particular aspects, the MBV are prepared following digestion of an ECM with an enzyme, such as pepsin, elastase, hyaluronidase, proteinase K, salt solutions, or collagenase. The ECM can be freeze-thawed, or subject to mechanical degradation.

In some aspects, expression of CD63, CD81, and/or CD9 cannot be detected on the MBV. Thus, in some aspects the MBV do not express CD63 and/or CD81 and/or CD9. In one specific example, CD63, CD81, and CD9 cannot be detected on the nanovesicles. In other aspects, the MBV have barely detectable levels of CD63, CD81, and CD9, such as that detectable by Western blot. These MBV are CD63^{lo}CD81^{lo}CD9^{lo}. In other aspects, MBV do not express detectable levels of one or more of CD63, CD81, or CD9. In other aspects, MBV do not express detectable levels of CD63 and CD81, or are CD63^{lo}CD81^{lo}. In other aspects, MBV express barely detectable levels of one or more of CD63, CD81, or CD9. One of skill in the art can readily identify MBV that are CD63^{lo} and/or CD81^{lo} and/or CD9^{lo}, using, for example, antibodies that specifically bind CD63, CD81, and CD9. A low level of these markers can be established using procedures such as fluorescent activated cell sorting (FACS) and fluorescently labeled antibodies to determine a threshold for low and high amounts of CD63, CD81, and CD9. The disclosed MBV differ from nanovesicles, such as exosomes that may be transiently attached to the surface of the ECM due to their presence in biological fluids, as MBV in vivo are bound to the ECM and not found in biological fluids.

MBV have distinctive phospholipid content, for example, in comparison to exosomes. In some aspects, the total phospholipid content of the MBV is at least 50%, 55%, 60%, 65%, 70%, 75%, 85%, or 90%, or about 50%-90%, 50%-65%, 50%-60%, 50%-70%, 60%-70%, 60%-90%, or 70%-90% of phosphatidylcholine (PC) and phosphatidyl inositol (PI) in combination. In specific aspects, the total phospholipid content of the MBV is at least 55% of phosphatidylcholine (PC) and phosphatidyl inositol (PI) in combination. In specific aspects, the total phospholipid content of the MBV is at least 60% of phosphatidylcholine (PC) and phosphatidyl inositol (PI) in combination. In some aspects, the phospholipid content of the MBV comprises a phosphatidylcholine (PC) to phosphatidyl inositol (PI) ratio of less than 8:1 (for example, less than 7:1, less than 6:1, less than 5:1, less than 4:1, less than 3:1, or less than 2:1). In some aspects, the phospholipid content of the MBV comprises a phosphatidylcholine (PC) to phosphatidyl inositol (PI) ratio in the range of 0.5-1:1, or in the range of 1:0.5-1, or in the range of 0.5-1:2, or in the range of 2:0.5-1, or in the range of 0.8-1:1, or in the range of 1:0.8-1. In one aspect, the phospholipid content of the MBV comprises a phosphatidylcholine (PC) to phosphatidyl inositol (PI) ratio of about 1:1. In specific aspects, the phospholipid content of the MBV comprises a phosphatidylcholine (PC) to phosphatidyl inositol (PI) ratio of about 0.9:1.

In some aspects, the total phospholipid content of the MBV is 15%, 10%, 9%, 8%, 7%, 6%, 5%, 4% or less, or about 5%-10%, 5%-15%, 10%-15%, or 8%-12% of sphingomyelin (SM). In specific aspects, the total phospholipid content of the MBV is 10% or less of sphingomyelin (SM). In some aspects, the total phospholipid content of the is 15% or less of sphingomyelin (SM), 14% or less of sphingomyelin, 13% or less of sphingomyelin, 12% or less of sphingomyelin, 11% or less of sphingomyelin, 10% or less of sphingomyelin, 9% or less of sphingomyelin, 8% or less of sphingomyelin, 7% or less of sphingomyelin, 6% or less of sphingomyelin, 5% or less of sphingomyelin, or 4% or less of sphingomyelin.

In some aspects, the total phospholipid content of the MBV 20%, 19%, 18%, 17%, 16%, 15%, 14%, 13%, 12%, 11%, or 10% or less, or about 10%-20%, 15%-20%, 14%-18%, or 12%-16% of phosphatidylethanolamine (PE). In specific aspects, the total phospholipid content of the MBV is 20% or less of phosphatidylethanolamine (PE).

In some aspects, the total phospholipid content of the MBV is 5%, 10%, 12%, 15%, 18%, 20%, 25%, or 30% or greater, or about 5%-30%, 10%-20%, 10-25%, 15%-25%, or 12%-18% of phosphatidylinositol (PI). In specific aspects, MBV include a phospholipid content 15% or greater of phosphatidylinositol (PI).

In specific aspects, the total phospholipid content of the MBV comprises 15% or more phosphatidylinositol, 20% or less phosphatidylethanolamine, and 10% or less sphingomyelin. In specific aspects, the total phospholipid content of the MBV is 15% or more phosphatidylinositol and 20% or less phosphatidylethanolamine. In specific aspects, the total phospholipid content of the MBV is 15% or more phosphatidylinositol and 10% or less sphingomyelin. In specific aspects, the total phospholipid content of the MBV comprises 20% or less phosphatidylethanolamine and 10% or less sphingomyelin. In specific aspects, the total phospholipid content of the MBV is more than 15% phosphatidylinositol, 20% or less phosphatidylethanolamine, 10% or less sphingomyelin, and at least 55% of phosphatidylinositol and phosphatidylcholine in combination. In one aspect, the total phospholipid content of the MBV is at least 55% phosphatidylcholine (PC) and phosphatidyl inositol (PI) in combination and 10% or less sphingomyelin (SM). In specific aspects, the total phospholipid content of the MBV is at least 55% of phosphatidylinositol and phosphatidylcholine in combination and more than 15% phosphatidylinositol. In specific aspects, the total phospholipid content of the MBV is 55% of phosphatidylinositol and phosphatidylcholine in combination and 20% or less phosphatidylethanolamine.

The MBV may also comprise lysyl oxidase (Lox). Generally, nanovesicles derived from the ECM have a higher Lox content than exosomes. Lox is expressed on the surface of MBV. Nano-LC MS/MS proteomic analysis can be used to detect Lox proteins. Quantification of Lox can be performed (see, e.g., Hill R C, et al., Mol Cell Proteomics. 2015; 14(4):961-73, incorporated herein by reference in its entirety).

In certain aspects, the MBV comprise one or more miRNA. In specific non-limiting examples, the MBV comprise one, two, or all three of miR-143, miR-145 and miR-181. MiR-143, miR-145 and miR-181 are known in the art. In some aspects, the MBV comprise as miR-145 and miR-181, for example, as cargo inside the vesicles.

The miR-145 nucleic acid sequence is provided in MiRbase Accession No. MI0000461, incorporated herein by reference. A miR-145 nucleic acid sequence is CACCUUGUCCUCACGGUCCAGUUUUCCCAGGAAUCCCUUAGAUGCUAAGAUGGGGA UUCCUGGAAAUACUGUUCUUGAGGUCAUGGUU (SEQ ID NO: 1). An miR-181 nucleic acid sequence is provided in miRbase Accession No. MI0000269, incorporated herein by reference. A miR-181 nucleic acid sequence is: AGAAGGGCUAUCAGGCCAGCCUUCAGAGGACUCCAAGGAACAUUCAACGCUGUCGG UGAGUUUGGGAUUUGAAAAAACCACUGACCGUUGACUGUACCUUGGGGUCCUUA (SEQ ID NO: 2). The miR-143 nucleic acid sequence is provided in NCBI Accession No. NR_029684.1, Mar. 30, 2018, incorporated herein by reference. A DNA encoding an miR-143 nucleic acid sequence is: GCGCAGCGCC CTGTCTCCCA GCCTGAGGTG CAGTGCTGCA TCTCTGGTCA GTTGGGAGTC TGAGATGAAG CACTGTAGCT CAGGAAGAGA GAAGTTGTTC TGCAGC (SEQ ID NO: 3).

Following administration, the MBV maintain expression of F4/80 (a macrophage marker) and CD-11b on macrophages in the subject. Nanovesicle treated macrophages are predominantly F4/80+Fizz1+indicating an M2 phenotype.

The MBV disclosed herein can be formulated into compositions for pharmaceutical delivery. MBV are further disclosed and described in PCT Publication No. WO 2017/151862, which is incorporated herein by reference.

Isolation of MBV from the ECM

To produce MBV, ECM can be produced by any cells of interest, or can be utilized from a commercial source, as described supra. The MBV can be produced from the same species as, or a different species than, the subject being treated. In some aspects, these methods include digesting the ECM with an enzyme to produce digested ECM. In specific aspects, the ECM is digested with one or more of pepsin, elastase, hyaluronidase, collagenase a metalloproteinase, and/or proteinase K. In a specific non-limiting example, the ECM is digested with only elastase and/or a metalloproteinase. In another non-limiting example, the ECM is not digested with collagenase and/or trypsin and/or proteinase K. In other aspects, the ECM is treated with a detergent. In further aspects, the method does not include the use of enzymes. In specific non-limiting examples, the method utilizes chaotropic agents or ionic strength to isolate MBV such as salts, such as potassium chloride. In additional aspects, the ECM can be manipulated to increase MBV content prior to isolation of MBV. Techniques for isolating MBV from ECM are described, for example, in International Patent Application WO 2017/151862 and Quijano et al., (2020), Tissue Eng Part C Methods, 26(10):528-540.

In some aspects, the ECM is digested with an enzyme. The ECM can be digested with the enzyme for about 12 to about 48 hours, such as about 12 to about 36 hours. The ECM can be digested with the enzyme for about 12, about 24 about 36 or about 48 hours. In one specific non-limiting example, the ECM is digested with the enzyme at room temperature. However, the digestion can occur at about 4° C., or any temperature between about 4° C. and 25° C. Generally, the ECM is digested with the enzyme for any length of time, and at any temperature, sufficient to remove collagen fibrils. The digestion process can be varied depending on the tissue source. Optionally, the ECM is processed by freezing and thawing, either before or after digestion with the enzyme. The ECM can be treated with detergents, including ionic and/or non-ionic detergents.

The digested ECM is then processed, such as by centrifugation, to isolate a fibril-free supernatant. In some aspects the digested ECM is centrifuged, for example, for a first step at about 300 to about 1000 g. Thus, the digested ECM can be centrifuged at about 400 g to about 750 g, such as at about 400 g, about 450 g, about 500 g or about 600 g. This centrifugation can occur for about 10 to about 15 minutes, such as for about 10 to about 12 minutes, such as for about 10, about 11, about 12, about 14, about 14, or about 15 minutes. The supernatant including the digested ECM is collected.

In some aspects, the MBV comprise Lox. In some aspects, methods for isolating such MBV include digesting the extracellular matrix with elastase and/or metalloproteinase to produce digested extracellular matrix, centrifuging the digested extracellular matrix to remove collagen fibril remnants and thus to produce a fibril-free supernatant, centrifuging the fibril-free supernatant to isolate the solid materials, and suspending the solid materials in a carrier.

In some aspects, digested ECM also can be centrifuged for a second step at about 2000 g to about 3000 g. Thus, the digested ECM can be centrifuged at about 2,500 g to about 3,000 g, such as at about 2,000 g, 2,500 g, 2,750 g or 3,000 g. This centrifugation can occur for about 20 to about 30 minutes, such as for about 20 to about 25 minutes, such as for about 20, about 21, about 22, about 23, about 24, about 25, about 26, about 27, about 28, about 29 or about 30 minutes. The supernatant including the digested ECM is collected.

In additional aspects, the digested ECM can be centrifuged for a third step at about 10,000 to about 15,000 g. Thus, the digested ECM can be centrifuged at about 10,000 g to about 12,500 g, such as at about 10,000 g, 11,000 g or 12,000 g. This centrifugation can occur for about 25 to about 40 minutes, such as for about 25 to about 30 minutes, for example for about 25, about 26, about 27, about 28, about 29, about 30, about 31, about 32, about 33, about 34, about 35, about 36, about 37, about 38, about 39 or about 40 minutes. The supernatant including the digested ECM is collected. One, two or all three of these centrifugation steps can be independently utilized. In some aspects, all three centrifugation steps are utilized. The centrifugation steps can be repeated, such as 2, 3, 4, or 5 times. In one aspect, all three centrifugation steps are repeated three times.

In some aspects, the digested ECM is centrifuged at about 500 g for about 10 minutes, centrifuged at about 2,500 g for about 20 minutes, and/or centrifuged at about 10,000 g for about 30 minutes. These step(s), such as all three steps are repeated 2, 3, 4, or 5 times, such as three times. Thus, in one non-limiting example, the digested ECM is centrifuged at about 500 g for about 10 minutes, centrifuged at about 2,500 g for about 20 minutes, and centrifuged at about 10,000 g for about 30 minutes. These three steps are repeated three times. Thus, a fibril-free supernatant is produced. The fibril-free supernatant is then centrifuged to isolate the MBV. In some aspects, the fibril-free supernatant is centrifuged at about 100,000 g to about 150,000 g. Thus, the fibril-free supernatant is centrifuged at about 100,000 g to about 125,000 g, such as at about 100,000 g, about 105,000 g, about 110,000 g, about 115,000 g or about 120,000 g. This centrifugation can occur for about 60 to about 90 minutes, such as about 70 to about 80 minutes, for example for about 60, about 65, about 70, about 75, about 80, about 85 or about 90 minutes. In one non-limiting example, the fiber-free supernatant is centrifuged at about 100,000 g for about 70 minutes. The solid material is collected, which is the MBV. These MBV then can be re-suspended in any carrier of interest, such as, but not limited to, a buffer.

In further aspects the ECM is not digested with an enzyme. In these methods, ECM is suspended in an isotonic saline solution, such as phosphate buffered saline. Salt is then added to the suspension so that the final concentration of the salt is greater than about 0.1 M. The concentration can be, for example, up to about 3 M, for example, about 0.1 M salt to about 3 M, or about 0.1 M to about 2M. The salt can be, for example, about 0.1M, 0.15M, 0.2M, 0.3M, 0.4 M, 0.7 M, 0.6 M, 0.7 M, 0.8M, 0.9M, 1.0 M, 1.1 M, 1.2 M, 1.3 M, 1.4 M, 1.5M, 1.6 M, 1.7 M, 1.8M, 1.9 M, or 2M. In some non-limiting examples, the salt is potassium chloride, sodium chloride or magnesium chloride. In other aspects, the salt is sodium chloride, magnesium chloride, sodium iodide, sodium thiocyanate, a sodium salt, a lithium salt, a cesium salt or a calcium salt.

In some aspects, the ECM is suspended in the salt solution for about 10 minutes to about 2 hours, such as about 15 minutes to about 1 hour, about 30 minutes to about 1 hour, or about 45 minutes to about 1 hour. The ECM can be suspended in the salt solution for about 15, 20, 25, 30, 35, 40, 45, 50, 55, 60, 65, 70, 75, 80, 85, 90, 95, 100, 105, 110, 115 or 120 minutes. The ECM can be suspended in the salt solution at temperatures from 4° C. to about 50° C., such as, but not limited to about 4° C. to about 25° C. or about 4° C. to about 37° C. In a specific non-limiting example, the ECM is suspended in the salt solution at about 4° C. In other specific non-limiting examples, the ECM is suspended in the salt solution at about 22° C. or about 25° C. (room temperature). In further non-limiting examples, the ECM is suspended in the salt solution at about 37° C.

In some aspects, the method includes incubating an extracellular matrix at a salt concentration of greater than about 0.4 M; centrifuging the digested extracellular matrix to remove collagen fibril remnants, and isolating the supernatant; centrifuging the supernatant to isolate the solid materials; and suspending the solid materials in a carrier, thereby isolating MBV from the extracellular matrix.

Following incubation in the salt solution, the ECM is centrifuged to remove collagen fibrils. In some aspects, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes. The supernatant is then collected.

In additional aspects, the supernatant then can be centrifuged for a third step at about 100,000 to about 150,000 g. Thus, the digested ECM can be centrifuged at about 100,000 g to about 125,000 g, such as at about 100,000 g, 110,000 g or 120,000 g. This centrifugation can occur for about 30 minutes to about 2.5 hour, such as for about 1 hour to about 3 hours, for example for about 30 minutes, about 45 minutes, about 60 minutes, about 90 minutes, or about 120 minutes (2 hours). The solid materials are collected and suspended in a solution, such as buffered saline, thereby isolating the MBV.

In yet other aspects, the ECM is suspended in an isotonic buffered salt solution, such as, but not limited to, phosphate buffered saline. Centrifugation or other methods can be used to remove large particles (see below). Ultrafiltration is then utilized to isolate MBV from the ECM, particles between about 10 nm and about 10,000 nm, such as between about 10 and about 1,000 nm, such as between about 10 nm and about 300 nm.

In specific non-limiting examples, the isotonic buffered saline solution has a total salt concentration of about 0.164 mM, and a pH of about 7.2 to about 7.4. In some aspects, the isotonic buffered saline solution includes 0.002 M KCl to about 0.164 M KCL, such as about 0.0027 M KCl (the concentration of KCL in phosphate buffered saline). This suspension is then processed by ultracentrifugation.

Following incubation in the isotonic buffered salt solution, the ECM is centrifuged to remove collagen fibrils. In some aspects, digested ECM also can be centrifuged at about 2000 g to about 5000 g. Thus, the digested ECM can be centrifuged at about 2,500 g to about 4,500 g, such as at about 2,500 g, about 3,000 g, 3,500, about 4,000 g, or about 4,500 g. In one specific non-limiting example, the centrifugation is at about 3,500 g. This centrifugation can occur for about 20 to about 40 minutes, such as for about 25 to about 35 minutes, such as for about 20, about 21, about 22, about 23, about 24, about 25, about 26, about 27, about 28, about 29, about 30 minutes, about 31, about 32, about 33 about 34 or about 35 minutes.

Microfiltration and centrifugation can be used and combined to remove large molecular weight materials from the suspension. In one aspect, large size molecule materials, such as more than 200 nm are removed using microfiltration. In another aspect, large size materials are removed by the use of centrifugation. In a third aspect both microfiltration and ultracentrifugation are used to remove large molecular weight materials. Large molecular weight materials are removed from the suspended ECM, such as materials greater than about 10,000 nm, greater than about 1,000 nm, greater than about 500 nm, or greater than about 300 nm.

The effluent for microfiltration or the supernatant is then subjected to ultrafiltration. Thus, the effluent, which includes particle of less than about 10,000 nm, less than about 1,000 nm, less than about 500 nm, or less than about 300 nm is collected and utilized. This effluent is then subjected to ultrafiltration with a membrane with a molecular weight cutoff (MWCO) of 3,000 to 100,000. 100,000 MWCO was used in the example.

### Immunogenic Compositions

Pharmaceutical compositions are provided that include isolated mammalian extracellular matrix bound vesicles (MBV) that do not express CD63 and CD81 or are CD63^{lo}CD81^{lo}, and an effective amount of a vaccine comprising or encoding a vaccine antigen, and a pharmaceutically acceptable carrier are also provided. In some aspects, the immunogenic composition also includes a vaccine adjuvant. In some embodiments, the pharmaceutical composition includes a cytokine, such as IL-12. These pharmaceutical compositions are of use in the methods disclosed herein.

In some aspects, the vaccine is a subunit vaccine, and thus incudes a vaccine antigen. The antigen can be, for example, a viral antigen, a bacterial antigen, a fungal antigen, or a tumor-associated antigen.

In other aspects, the vaccine includes an mRNA encoding the vaccine antigen. In specific, non-limiting examples, the mRNA encodes a viral protein, a bacterial protein, a fungal protein, a parasitic protein, or a tumor associated protein.

In other aspects, the vaccine is a live attenuated vaccine. In specific non-limiting examples, the vaccine is a live attenuated virus, bacteria, fungus, or parasite.

In further aspects, the vaccine is an inactivated vaccine. In specific non-limiting examples, the vaccine is an inactivated virus, bacteria, fungus, or parasite. The vaccine can be a heat killed vaccine or chemically inactivated vaccine

Generally, any molecule or portion of a molecule against which an immune response is desired may be used as a vaccine antigen. The vaccine can include any one of, but not limited to, peptides, polypeptides, proteins, cells (or components thereof), live-attenuated pathogens (or components thereof), and heat-killed pathogens (or components thereof).

Such compositions can be administered to subjects by a variety of administration modes, for example, intramuscular, subcutaneous, intravenous, intra-arterial, intra-articular, intraperitoneal, or parenteral routes. Methods for preparing administrable compositions are described in more detail in such publications as Remington: The Science and Practice of Pharmacy, 22^{nd} ed., London, UK: Pharmaceutical Press, 2013.

Vaccine antigens may be derived from a pathogen, e.g., a bacteria, a virus, a parasite, or a fungus, or from a tumor, e.g., a tumor-associated antigen. In some aspects, such immunogens are administered, e.g., in a vaccine in combination with MBV.

### Bacterial Pathogens

In some aspects, the vaccine antigen includes an antigen from a bacterium. Such a vaccine antigen may be a bacterial protein or portion thereof. The protein may be encoded by an mRNA and expressed upon administration to a subject. Specific examples of bacterial pathogens include without limitation any one or more of (or any combination of) Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae, Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophila psittaci, Citrobacter sp. Coxiella burnetii, Corynebacterium sp. (such as, Corynebacterium diphtheriae, Corynebacterium jeikeum and Corynebacterium), Clostridium sp. (such as Clostridium perfringens, Clostridium difficile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli) Enterococcus sp. (such as Enterococcus faecalis and Enterococcus faecium) Ehrlichia sp. (such as Ehrlichia chafeensia and Ehrlichia canis), Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus, Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Moraxella catarrhalis, Morganella sp., Mobiluncus sp., Micrococcus sp., Mycobacterium sp. (such as Mycobacterium leprae, Mycobacterium tuberculosis, Mycobacterium intracellulare, Mycobacterium avium, Mycobacterium bovis, and Mycobacterium marinum), Mycoplasma sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia among others.

Bacterial antigens suitable for use in a vaccine include proteins, polysaccharides, lipopolysaccharides, and outer membrane vesicles which may be isolated, purified or derived from a bacterium. In addition, bacterial antigens include bacterial lysates and inactivated bacteria formulations. Bacteria antigens can be produced by recombinant expression. Bacterial antigens preferably include epitopes which are exposed on the surface of the bacteria during at least one stage of its life cycle. Bacterial antigens include but are not limited to antigens derived from one or more of the bacteria set forth above as well as the specific antigens examples identified below.

In some aspects, the vaccine antigen is a gram positive bacterial antigen. For example, the vaccine antigen can be a lipoteichoic acid (LTA). In other aspects, the vaccine antigen is a gram negative bacterial antigen. For example, the vaccine antigen can be a lipopolysaccharide (LPS).

Neisseria gonorrhoeae antigens include Por (or porin) protein, such as PorB (see, e.g., Zhu et al. (2004) Vaccine 22:660-669), a transferring binding protein, such as TbpA and TbpB (see, e.g., Price et al. (2004) Infect. Immun. 71(1):277-283), an opacity protein (such as Opa), a reduction-modifiable protein (Rmp), and outer membrane vesicle (OMV) preparations (see, e.g., Plante et al. (2000) J. Infect. Dis. 182:848-855); WO 99/24578; WO 99/36544; WO 99/57280; and WO 02/079243, all of which are incorporated by reference).

Chlamydia trachomatis antigens include antigens derived from serotypes A, B, Ba and C (agents of trachoma, a cause of blindness), serotypes Li, L3 (associated with Lymphogranuloma venereum), and serotypes, D-K. Chlamydia trachomas antigens also include antigens identified in WO 00/37494; WO 03/049762; WO 03/068811; and WO 05/002619 (all of which are incorporated by reference), including PepA (CT045), LcrE (CT089), Art (CT381), DnaK (CT396), CT398, OmpH-like (CT242), L7/L12 (CT316), OmcA (CT444), AtosS (CT467), CT547, Eno (CT587), HrtA (CT823), MurG (CT761), CT396 and CT761, and specific combinations of these antigens.

Treponemapallidum (Syphilis) antigens include TmpA antigen.

### Viral Pathogens

In some aspects, the vaccine antigen comprises an antigen derived from a virus. Such a vaccine antigen may be a viral protein or portion thereof. The protein may be encoded by an mRNA and expressed upon administration to a subject. Exemplary viruses include, but are not limited to, Avian herpesviruses, Bovine herpesviruses, Canine herpesviruses, Equine herpesviruses, herpes simplex virus-1 (HSV-1), herpes simplex virus-2 (HSV-2), Feline viral rhinotracheitis virus, Marek's disease virus, Ovine herpesviruses, Porcine herpesviruses, Pseudorabies virus, Avian paramyxoviruses, Bovine respiratory syncytial virus, Human respiratory syncytial virus (RSV), Canine distemper virus, Canine parainfluenza virus, canine adenovirus, canine parvovirus, monkeypox virus, Bovine Parainfluenza virus 3, Ovine parainfluenza 3, human parainfluenza, Rinderpest virus, Border disease virus, Bovine viral diarrhea virus (BVDV), BVDV Type I, BVDV Type II, Chikungunya virus, Classical swine fever virus, cytomegalovirus (CMV), Avian Leukosis virus, Bovine immunodeficiency virus, Bovine leukemia virus, Bovine tuberculosis, Ebola virus, Epstein Barr Virus (EBV), Equine infectious anemia virus, Feline immunodeficiency virus, Feline leukemia virus (FeLV), Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, virus, Porcine epidemic diarrhea virus, Porcine hemagglutinating encephalomyelitis virus, Porcine parvovirus, Porcine Circovirus (PCV) Type I, PCV Type II, Porcine Reproductive and Respiratory Syndrome (PRRS) Virus, Transmissible gastroenteritis virus, Turkey coronavirus, Bovine ephemeral fever virus, Rabies virus, Rotavirus, small pox (variola) virus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, severe acute respiratory syndrome coronavirus 1 (SARS-COV-1), severe acute respiratory syndrome coronavirus 2 (SARS-COV-2), a coronavirus, zika virus, a paramyxovirus, polio virus, hepatitis A virus, hepatitis C virus, Rubella virus, human parvovirus, norovirus, mumps virus, molluscum contagiosum virus, Rubeola virus, enterovirus, coxsackievirus, a picornavirus, a herpesvirus and combinations thereof.

### Fungal Pathogens

In some aspects, the vaccine antigen includes an antigen derived from a fungus. Such a vaccine antigen may be a fungal protein or portion thereof. The protein may be encoded by an mRNA and expressed upon administration to a subject. Exemplary fungal pathogens include one or more of Trichophyton rubrum, T. mentagrophytes, Epidermophyton floccosum, Microsporum canis, Pityrosporum orbiculare (Malassezia furfur), Candida sp. (such as Candida albicans), Aspergillus sp. (such as Aspergillus fumigatus, Aspergillus flavus and Aspergillus clavatus), Cryptococcus sp. (such as Cryptococcus neoformans, Cryptococcus gattii, Cryptococcus laurentii and Cryptococcus albidus), Histoplasma sp. (such as Histoplasma capsulatum), Pneumocystis sp. (such as Pneumocystis jirovecii), and Stachybotrys (such as Stachybotrys chartarum).

### Parasites

In some aspects, the vaccine includes a vaccine antigen is derived from a parasite. Such a vaccine antigen may be a parasitic protein or portion thereof. The protein may be encoded by an mRNA and expressed upon administration to a subject. Exemplary parasitic organisms include Malaria (Plasmodium falciparum, P. vivax, P. malariae), Schistosomes, Trypanosomes, Leishmania, Filarial nematodes, Trichomoniasis, Sarcosporidiasis, Taenia (T. saginata, T. solium), Leishmania, Toxoplasma gondii, Trichinelosis (Trichinella spiralis) or Coccidiosis (Eimeria species).

### Tumor Antigens

In some aspects, the vaccine antigen includes an antigen derived from a tumor, e.g., a tumor associated antigen. Such a vaccine antigen may be a protein or a portion thereof. Exemplary tumor associated antigens include one or more of the following: RAGE-1, tyrosinase, MAGE-1, MAGE-2, NY-ESO-1, Melan-A/MART-1, glycoprotein (gp) 75, gp100, beta-catenin, preferentially expressed antigen of melanoma (PRAME), MUM-1, Wilms tumor (WT)-1, carcinoembryonic antigen (CEA), and PR-1. Additional tumor antigens are known in the art (for example see Novellino et al., Cancer Immunol. Immunother. 54(3):187-207, 2005) and are described below. Tumor antigens are also referred to as “cancer antigens.” The tumor antigen can be any tumor-associated antigen, which are well known in the art and include, for example, carcinoembryonic antigen (CEA), b-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, macrophage colony stimulating factor, prostase, prostate-specific antigen (PSA), PAP, NY-ESO-1, LAGE-1a, p53, prostein, PSMA, Her2/neu, survivin and telomerase, prostate-carcinoma tumor antigen-1, MAGE, ELF2M, neutrophil elastase, ephrinB2, CD22, insulin growth factor (IGF)-I, IGF-II, IGF-I receptor and mesothelin. A list of selected tumor antigens and their associated tumors are shown below in Table 1.

TABLE 1
Exemplary tumors and representative tumor antigens

| Tumor | Tumor Associated Target Antigens |
|---|---|
| Acute myelogenous | Wilms tumor 1 (WT1), PRAME, PR1, |
| leukemia | proteinase 3, elastase, cathepsin G |
| Chronic myelogenous | WT1, PRAME, PR1, proteinase 3, elastase, |
| leukemia | cathepsin G |
| Myelodysplastic | WT1, PRAME, PR1, proteinase 3, elastase, |
| syndrome | cathepsin G |
| Acute lymphoblastic | PRAME |
| leukemia | |
| Chronic lymphocytic | Survivin |
| leukemia | |
| Non-Hodgkin's | Survivin |
| lymphoma | |
| Multiple myeloma | NY-ESO-1 |
| Malignant melanoma | MAGE, MART, Tyrosinase, PRAME |
| | GP100 |
| Breast cancer | WT1, Herceptin, epithelial tumor antigen |
| | (ETA) |
| Lung cancer | WT1 |
| Ovarian cancer | CA-125 |
| Prostate cancer | PSA |
| Pancreatic cancer | CA19-9, RCAS1 |
| Colon cancer | CEA |
| Cervical Cancer | SCC, CA125, CEA, Cytokeratins (TPA, |
| | TPS, Cyfra21-1) |
| Renal cell carcinoma | Fibroblast growth factor 5 |
| (RCC) | |
| Germ cell tumors | AFP |

A vaccine antigen as described herein may be the naturally occurring form of the antigen as derived from its natural source. The naturally occurring antigens may also be converted to other forms, including less toxic forms, which may be fragments or may contain other deletions, additions or modifications. These converted forms of antigens generally will retain immunogenicity. Diphtheria and tetanus toxoids are examples of detoxified forms of natural antigens, in this case produced by chemical (e.g., formaldehyde) treatment. Other means for eliminating toxicity of antigens are well known and include enzymatic digestion/fragmentation of protein antigens, denaturation (commonly through heat or chemical treatment), conjugation, chemical modification, and others. A vaccine antigen can be a recombinant antigen that does not occur in nature.

In some aspects, multiple antigens are administered in a single vaccine formulation to induce protection against multiple diseases, infectious agents, types, serotypes, serovars, and others, and the compositions of the present disclosure may similarly include multiple antigens. Particular examples of such antigens which are combined include diphtheria, tetanus, pertussis and other antigens. In some aspects, antigens may also be associated with a carrier protein that mediates the immunogenicity of the antigens. Examples of such conjugated antigens are well known in the art and commercially available in pharmaceutical formulations as vaccines.

The concentration of the antigen in the composition may be of any concentration, but generally is sufficient to stimulate an immune system when administered to an individual or mammal. In some aspects, the concentration of the one or more antigens is 10 μg per ml. In other examples, the concentration of one or more antigens may be 20, 30, 40, 50, 100, 150, 200, 250, 300, 350, 400, 450, 500, 550, 600, 650, 700, 750, 800, 850, 900, 950, or 1000 μg/ml. In some aspects, the concentration of antigen may be 2, 3, 4, 5, 6, 7, 8, 9, 10 mg/ml or even more. The concentration of the one or more antigens may also be in a range between any two of the values listed above.

In some aspects, MBV are administered with a vaccine, where said vaccine is one of the vaccines listed in Table 2 below.

TABLE 2
United States Food and Drug Administration (FDA) authorized vaccines

| | Trade Name |
|---|---|
| Product Name | (Manufacturer) |
| Adenovirus Type 4 and Type 7 Vaccine, Live, Oral | No Trade Name |
| | (Barr Labs) |
| Anthrax Vaccine Adsorbed | Biothrax ® |
| | (Emergent |
| | BioDefense Operations |
| | Lansing) |
| Bacille Calmette-Guérin (BCG) Live | BCG Vaccine |
| | (Organon Teknika |
| | Corp.) |
| BCG Live | TICE ® BCG |
| | (Organon Teknika |
| | Corp.) |
| Cholera Vaccine Live Oral | Vaxchora ® |
| | (Emergent Travel |
| | Health) |
| COVID-19 Vaccine, mRNA | Comirnaty ® |
| | (Pfizer-BioNTech) |
| COVID-19 Vaccine, mRNA | SPIKEVAX ® |
| | (Moderna) |
| COVID-19 Vaccine, protein subunit | NUVAXOVID ®, |
| | COVOVAX ® |
| | (Novovax) |
| Dengue Tetravalent Vaccine, Live | DENGVAXIA ® |
| | (Sanofi Pasteur) |
| Diphtheria & Tetanus Toxoids Adsorbed | No Trade Name |
| | (Sanofi Pasteur) |
| Diphtheria & Tetanus Toxoids & Acellular Pertussis Vaccine | Infanrix ® |
| Adsorbed | (GlaxoSmithKline) |
| Diphtheria & Tetanus Toxoids & Acellular Pertussis Vaccine | DAPTACEL ® |
| Adsorbed | (Sanofi Pasteur) |
| Diphtheria & Tetanus Toxoids & Acellular Pertussis Vaccine | Pediarix ™ |
| Adsorbed, Hepatitis B (recombinant) and Inactivated Poliovirus | (GlaxoSmithKline) |
| Vaccine Combined | |
| Diphtheria and Tetanus Toxoids and Acellular Pertussis | KINRIX ™ |
| Adsorbed and Inactivated Poliovirus Vaccine | (GlaxoSmithKline) |
| Diphtheria and Tetanus Toxoids and Acellular Pertussis | Quadracel ® |
| Adsorbed and Inactivated Poliovirus Vaccine | (Sanofi Pasteur) |
| Diphtheria and Tetanus Toxoids and Acellular Pertussis | VAXELIS ® |
| Adsorbed, Inactivated Poliovirus, Haemophilus b Conjugate | (MSP Vaccine |
| [Meningococcal Protein Conjugate] and Hepatitis B [Recombinant] | Company) |
| Vaccine | |
| Diphtheria and Tetanus Toxoids and Acellular Pertussis | Pentacel ™ |
| Adsorbed, Inactivated Poliovirus and Haemophilus b Conjugate | (Sanofi Pasteur) |
| (Tetanus Toxoid Conjugate) Vaccine | |
| Ebola Zaire Vaccine, Live | ERVEBO ® |
| | (Merck Sharp & |
| | Dohme) |
| Haemophilus b Conjugate Vaccine (Meningococcal Protein | PedvaxHIB ® |
| Conjugate) | (Merck Sharp & |
| | Dohme) |
| Haemophilus b Conjugate Vaccine (Tetanus Toxoid | ActHIB ® |
| Conjugate) | (Sanofi Pasteur) |
| Haemophilus b Conjugate Vaccine (Tetanus Toxoid | Hiberix ® |
| Conjugate) | (GlaxoSmithKline) |
| Hepatitis A Vaccine, Inactivated | Havrix ® |
| | (GlaxoSmithKline) |
| Hepatitis A Vaccine, Inactivated | VAQTA ® |
| | (Merck Sharp & |
| | Dohme) |
| Hepatitis A Inactivated and Hepatitis B (Recombinant) | Twinrix ® |
| Vaccine | (GlaxoSmithKline) |
| Hepatitis B Vaccine (Recombinant) | Recombivax HB ® |
| | (Merck & Co.) |
| Hepatitis B Vaccine (Recombinant) | PREHEVBRIO ® |
| | (VBI Vaccines) |
| Hepatitis B Vaccine (Recombinant) | Engerix-B |
| | (GlaxoSmithKline) |
| Hepatitis B Vaccine (Recombinant), Adjuvanted | HEPLISAV-B ® |
| | (Dynavax) |
| Human Papillomavirus Quadrivalent (Types 6, 11, 16, 18) | Gardasil ® |
| Vaccine, Recombinant | (Merck & Co.) |
| Human Papillomavirus 9-valent Vaccine, Recombinant | Gardasil ® 9 |
| | (Merck Sharp & |
| | Dohme) |
| Human Papillomavirus Bivalent (Types 16, 18) Vaccine, | Cervarix ® |
| Recombinant | (GlaxoSmithKline) |
| Influenza A (H1N1) 2009 Monovalent Vaccine | No Trade Name |
| | (CSL Limited) |
| Influenza A (H1N1) 2009 Monovalent Vaccine | No Trade Name |
| | (MedImmune) |
| Influenza A (H1N1) 2009 Monovalent Vaccine | No Trade Name |
| | (ID Biomedical |
| | Corporation of Quebec) |
| Influenza A (H1N1) 2009 Monovalent Vaccine | No Trade Name |
| | (Novartis Vaccines |
| | and Diagnostics) |
| Influenza A (H1N1) 2009 Monovalent Vaccine | No Trade Name |
| | (Sanofi Pasteur) |
| Influenza Virus Vaccine, H5N1 (for National Stockpile) | No Trade Name |
| | (Sanofi Pasteur) |
| Influenza A (H5N1) Virus Monovalent Vaccine, Adjuvanted” | No Trade Name |
| | (ID Biomedical |
| | Corporation of Quebec) |
| Influenza A (H5N1) Monovalent Vaccine, Adjuvanted | AUDENZ ™ |
| | (Seqirus) |
| Influenza Vaccine, Adjuvanted | FLUAD ® |
| | Quadrivalent |
| | (Seqirus) |
| Influenza Vaccine, Adjuvanted | Fluad ® |
| | (Seqirus) |
| Influenza Vaccine | AFLURIA ® |
| | Quadrivalent, AFLURIA ® |
| | Quadrivalent Southern |
| | Hemisphere |
| | (Seqirus) |
| Influenza Vaccine | FLUCELVAX ® |
| | Quadrivalent |
| | (Seqirus) |
| Influenza Virus Vaccine | AFLURIA ®, |
| (Trivalent, Types A and B) | AFLURIA ® Southern |
| | Hemisphere |
| | (Seqirus) |
| Influenza Virus Vaccine | FluLaval ® |
| (Trivalent, Types A and B) | (ID Biomedical |
| | Corporation of Quebec) |
| Influenza Vaccine, Live, Intranasal | FluMist ® |
| (Trivalent, Types A and B) | (MedImmune) |
| Influenza Virus Vaccine | Fluarix |
| (Trivalent, Types A and B) | (GlaxoSmithKline) |
| Influenza Virus Vaccine | Fluvirin |
| (Trivalent, Types A and B) | (Seqirus) |
| Influenza Virus Vaccine | Agriflu ® |
| (Trivalent, Types A and B) | (Seqirus) |
| Influenza Virus Vaccine | FLUZONE ®, |
| (Trivalent, Types A and B) | FLUZONE ® High-Dose |
| | and FLUZONE ® |
| | Intradermal |
| | (Sanofi Pasteur) |
| Influenza Virus Vaccine | Flucelvax ® |
| (Trivalent, Types A and B) | (Seqirus) |
| Influenza Virus (Trivalent) | Flublok ® |
| | (Protein Sciences |
| | Corporation) |
| Influenza Vaccine (Quadrivalent) | FLUBLOK ® |
| | Quadrivalent |
| | (Protein Sciences |
| | Corporation) |
| Influenza Vaccine, Live, Intranasal | FLUMIST ® |
| (Quadrivalent, Types A and Types B) | Quadrivalent |
| | (MedImmune) |
| Influenza Virus Vaccine (Quadrivalent, Types A and Types | FLUARIX ® |
| B) | Quadrivalent |
| | (GlaxoSmithKline) |
| Influenza Virus Vaccine | FLUZONE ® |
| (Quadrivalent, Types A and Types B) | Quadrivalent |
| | (Sanofi Pasteur) |
| Influenza Vaccine | FLULAVAL ® |
| | Quadrivalent |
| | (ID Biomedical |
| | Corporation of Quebec) |
| Japanese Encephalitis Virus Vaccine, Inactivated, Adsorbed | Ixiaro |
| | (Valneva Austria) |
| Measles, Mumps, and Rubella Virus Vaccine, Live | M-M-R ® II |
| | (Merck, Sharpe & |
| | Dohme) |
| Measles, Mumps, Rubella and Varicella Virus Vaccine Live | ProQuad ® |
| | (Merck, Sharpe & |
| | Dohme) |
| Meningococcal (Groups A, C, Y, and W-135) | Menveo |
| Oligosaccharide Diphtheria CRM197 Conjugate Vaccine | (GlaxoSmithKline) |
| Meningococcal (Groups A, C, Y and W-135) Polysaccharide | Menactra ® |
| Diphtheria Toxoid Conjugate Vaccine | (Sanofi Pasteur) |
| Meningococcal Group B Vaccine | BEXSERO ® |
| | (GlaxoSmithKline) |
| Meningococcal Group B Vaccine | TRUMENBA ® |
| | (Wyeth) |
| Meningococcal Polysaccharide Vaccine, Groups A, C, Y and | Menomune- |
| W-135 Combined (discontinued) | A/C/Y/W-135 |
| | (Sanofi Pasteur) |
| Meningococcal (Groups A, C, Y, W) Conjugate Vaccine | MenQuadfi ® |
| | (Sanofi Pasteur) |
| Plague Vaccine | No trade name |
| | (DynPort Vaccine) |
| Pneumococcal Vaccine, Polyvalent | Pneumovax ® 23 |
| | (Merck, Sharpe & |
| | Dohme) |
| Pneumococcal 13-valent Conjugate Vaccine | Prevnar ® 13 |
| (Diphtheria CRM₁₉₇ Protein) | (Wyeth) |
| Pneumococcal 15-valent Conjugate Vaccine | VAXNEUVANCE ™ |
| | (Merck, Sharpe & |
| | Dohme) |
| Pneumococcal 20-valent Conjugate Vaccine | Prevnar ® 20 |
| | (Wyeth) |
| Poliovirus Vaccine Inactivated (Human Diploid Cell) | Poliovax ® |
| | (discontinued) |
| IPOL - Poliovirus Vaccine Inactivated (Monkey Kidney Cell) | IPOL ® |
| | (Sanofi Pasteur) |
| Rabies Vaccine | |
| Rabies Vaccine | RabAvert |
| | (Novartis Vaccines |
| | and Diagnostics) |
| Rabies Vaccine Adsorbed | No Trade Name |
| Rotavirus Vaccine, Live, Oral | ROTARIX ® |
| | (GlaxoSmithKline) |
| Rotavirus Vaccine, Live, Oral, Pentavalent | RotaTeq ® |
| | (Merck, Sharpe & |
| | Dohme) |
| Smallpox and Monkeypox Vaccine, Live, Non-Replicating | JYNNEOS ® |
| | (Bavarian Nordic) |
| Smallpox (Vaccinia) Vaccine, Live | ACAM2000 ® |
| | (Emergent Product |
| | Development) |
| Tetanus & Diphtheria Toxoids, Adsorbed | TDVAX ® |
| | (MassBiologics) |
| Tetanus & Diphtheria Toxoids Adsorbed for Adult Use | TENIVAC ® |
| | (Sanofi Pasteur) |
| Diptheria and Tetanus Toxoids Adsorbed | No Trade Name |
| | (Sanofi Pasteur) |
| Tetanus Toxoid, Reduced Diphtheria Toxoid and Acellular | Adacel ® |
| Pertussis Vaccine, Adsorbed | (Sanofi Pasteur) |
| Tetanus Toxoid, Reduced Diphtheria Toxoid and Acellular | Boostrix |
| Pertussis Vaccine, Adsorbed | (GlaxoSmithKline) |
| Tick-Borne Encephalitis Vaccine | TICOVAC ™ |
| | (Pfizer) |
| Typhoid Vaccine Live Oral Ty21a | Vivotif ® |
| | (Berna Biotech) |
| Typhoid Vi Polysaccharide Vaccine | TYPHIM VI ® |
| | (Sanofi Pasteur) |
| Varicella Virus Vaccine Live | Varivax ® |
| | (Merck, Sharpe & |
| | Dohme) |
| Yellow Fever Vaccine | YF-Vax ® |
| | (Sanofi Pasteur) |
| Zoster Vaccine, Live, (Oka/Merck) (discontinued) | Zostavax |
| | (Merck & Co.) |
| Zoster Vaccine Recombinant, Adjuvanted | SHINGRIX |
| | (GlaxoSmithKline) |

### Stabilized RNA Molecules for Use

In some aspects, the vaccine is an mRNA encoding the vaccine antigen. In some aspects, a signal peptide is utilized. In some aspects, the endogenous signal peptide of a protein that is a vaccine antigen can be replaced with a heterologous signal peptide. In some aspects, the mRNA encodes the native signal peptide and does not encode a heterologous signal peptide. In other aspects, the mRNA encodes a heterologous signal peptide and a protein of interest that is a vaccine antigen.

In some aspects, a nucleic acid sequence encoding signal peptide can be 5′ to the nucleic acid sequence encoding the protein of interest, e.g., the immunogen. In other aspects, a nucleic acid sequence encoding signal peptide can be 3′ to the nucleic acid sequence encoding the protein of interest. Thus, in the encoded vaccine antigen, a heterologous signal peptide can be 5′ or 3′ to the protein of interest.

In some aspects, the mRNA encoding the protein of interest has 5′ and 3′ UTRs. In one aspect, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, the 5′ and 3′ UTR lengths can be modified as needed to increase translation efficiency following transfection of the transcribed RNA

The 5′ and 3′ UTRs can be the naturally occurring, endogenous 5′ and 3′ UTRs for the gene encoding the protein of interest. Alternatively, UTR sequences that are not endogenous to the gene of interest can be added by incorporating the UTR sequences into the forward and reverse primers or by any other modifications of the template. Without wishing to be bound by theory, the use of UTR sequences that are not endogenous to the gene of interest can be useful for modifying the stability and/or translation efficiency of the RNA. Without wishing to be bound by theory, AU-rich elements in 3′ UTR sequences can decrease the stability of mRNA. Therefore, 3′ UTRs can be selected or designed to increase the stability of the transcribed RNA based on properties of UTRs that are well known in the art.

In one aspect, the 5′ UTR can contain the Kozak sequence of the endogenous gene. Alternatively, in some aspects, when a 5′ UTR that is not endogenous to the gene of interest is used, a consensus Kozak sequence can be designed by adding the 5′ UTR sequence. Kozak sequences can increase the efficiency of translation of some RNA transcripts, but are not required for all RNAs to enable efficient translation.

Thus, in some aspects, the mRNAs that encode the protein of interest includes a 5′ UTR and/or a 3′ UTR that results in greater mRNA stability and higher expression of the mRNA in the cells.

In some embodiments, the mRNA includes a Kozak sequence in the 5′ UTR. The Kozak sequence can be, for example, ACCAUGG. This Kozak sequence can be included in any of the 5′ UTRs listed herein.

An exemplary 5′ UTR comprises, or consists of:

(SEQ ID NO: 4)
UCUCAACACAACAUAUACAAAACAAACGAAUCUCAAGCAAUCAAGCAUU
CUACUUCUAUUGCAGCAAUUUAAAUcauuucuuuuaaagcaaaagcaau
uuucugaaaauuuucaccauuuacgaacgau

In other aspects, the 5′ UTR comprises, or consists of

(SEQ ID NO: 5)
CGAACUAGUAUUCUUCUGGUCCCCACAGACUCAGAGAGAACCCGCCACC

In further aspects, the 5′UTR comprises, or consists of:

(SEQ ID NO: 6)
AGGAGGGUUUUUACC

In yet other aspects, the 3′ UTR comprises or consists of:

(SEQ ID NO: 7)
AACCAGCCUCAAGAACACCCGAAUGGAGUCUCUAAGCUACAUAAUACCA
ACUUACACUUACAAAAUGUUGUCCCCCAAAAUGUAGCCAUUCGUAUCUG
CUCCUAAUAAAAAGAAAGUUUCUU.

In some aspects, additional sequences, such as plasmid sequences, can be included.

In some aspects, the mRNA is polyadenylated. In some aspects, the mRNA comprises a poly-A tail (e.g., a poly-A tail having 50-200 nucleotides, such as 100-200, 150-200 nucleotides, or greater than 100 nucleotides), although in some aspects, a longer or a shorter poly-A tail is used. In some aspects, the poly-A tail is 100, 101, 102, 103, 104, 105, 106, 107, 108, 109, or 110 nucleotides in length.

The recombinant mRNA encoding the protein of interest can include a 5′ capping structure. 5′-capping of modified RNA can be completed concomitantly during IVT using the following chemical RNA cap analogs to generate the 5′-guanosine cap structure: 3′-O-Me-m7G (5′) ppp (5′) G; G (5′) ppp (5′) A; G (5′) ppp (5′) G; m7G (5′) ppp (5′) A; m7G (5′) ppp (5′) G (New England BioLabs, Ipswich, Mass.). In some aspects, 5′-capping of modified RNA may be completed post-transcriptionally using a Vaccinia Vims Capping Enzyme to generate the “Cap 0” structure: m7G (5′) ppp (5′) G (New England BioLabs, Ipswich, Mass.). Cap 1 structure can be generated using both Vaccinia ViJ.us Capping Enzyme and a 2′-0 methyl-transferase to generate: m7G (5′) ppp (5′) G-2′-O-methyl. Cap 2 structure can be generated from the Cap 1 structure followed by the 2′-O-methylation of the 5′-antepenultimate nucleotide using a 2′-0 methyl-transferase. Cap 3 structure can be generated from the Cap 2 structure followed by the 2′-O-methylation of the 5′-preantepenultimate nucleotide using a 2′-0 methyl-transferase. See U.S. Pat. No. 9,701,965, incorporated herein by reference.

To enable synthesis of RNA from a DNA template without the need for gene cloning, a promoter of transcription can be attached to the DNA template, upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one aspect, the promoter is a T7 RNA polymerase promoter, as described in U.S. Published Patent Application No. 2016/0030527A1, incorporated herein by reference. Other useful promoters include, but are not limited to, T3 and SP6 RNA polymerase promoters. Consensus nucleotide sequences for T7, T3 and SP6 promoters are known in the art.

The mRNA can be prepared using in vitro transcription (IVT). The IVT can be performed using any RNA polymerase as long as synthesis of the mRNA from the DNA template that encodes the RNA is specifically and sufficiently initiated from a respective cognate RNA polymerase promoter and full-length mRNA is obtained. In some preferred aspects, the RNA polymerase is T7 RNA polymerase, SP6 RNA polymerase or T3 RNA polymerase. In some other aspects, capped RNA is synthesized co-transcriptionally by using a dinucleotide cap analog in the IVT reaction (e.g., using an AMPLICAP™ T7 Kit or a MESSAGEMAX™ T7 ARCA-CAPPED MESSAGE Transcription Kit; EPICENTRE or CellScript, Madison, Wis., USA). If capping is performed co-transcriptionally, the dinucleotide cap analog can be an anti-reverse cap analog (ARCA). However, use of a separate IVT reaction, followed by capping with a capping enzyme system, which results in approximately 100% of the RNA being capped. Another option is co-transcriptional capping, which typically results in only about 80% of the RNA being capped. Thus, in some aspects, a high percentage of the mRNA molecules are capped (e.g., greater than 80%, greater than 90%, greater than 95%, greater than 98%, greater than 99%, greater than 99.5%, or greater than 99.9% of the population of mRNA molecules are capped).

In more aspects, the mRNA can be prepared by polyadenylation of an in vitro-transcribed (IVT) RNA using a poly(A) polymerase (e.g., yeast RNA polymerase or E. coli poly(A) polymerase). In some aspects, the mRNA is polyadenylated during in vitro transcription (IVT) by using a DNA template that encodes the poly(A) tail.

Poly(A) tails of RNAs can be further extended following in vitro transcription with the use of a poly(A) polymerase, such as E. coli polyA polymerase (E-PAP) or yeast polyA polymerase. In one aspect, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase.

An exemplary mRNA sequence of use in the disclosed methods includes, in 5′ to 3′ order, a Cap, a 5′UTR including a Kozak sequence, a codon optimized sequence encoding the protein of interest, such as the vaccine antigen, and a poly A tail. In this aspect, the RNA encodes the native signal peptide of the protein of interest.

In some aspects, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatemeric product which is not suitable for expression in eukaryotic cells. The transcription of plasmid DNA linearized at the end of the 3′ UTR results in normal sized mRNA which is effective in eukaryotic transfection when it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003). The conventional method of integration of polyA/T stretches into a DNA template is molecular cloning. If a polyA/T sequence integrated into plasmid DNA can cause plasmid instability in some cells, then this instability can be ameliorated through the use of recombination incompetent bacterial cells for plasmid propagation.

### Modified Nucleic Acids for RNA Molecules

In other aspects, vaccines described herein, e.g., vaccines for use in combination with MBV, may use recombinant mRNA encoding the protein of interest, including RNAs that contain one or more modified nucleosides (termed “modified nucleic acids”), which have useful properties including the lack of a substantial induction of the innate immune response of a cell into which the mRNA is introduced. As disclosed in U.S. Pat. No. 9,701,965, incorporated herein by reference, these modified nucleic acids enhance the efficiency of protein production, intracellular retention of nucleic acids, and viability of contacted cells, as well as possess reduced immunogenicity. Exemplary mRNA vaccinations include, for example and without limitation, the COMIRNATY® and SPIKEVAX® vaccines against SARS-COV-2. See Table 2.

In some aspects, a vaccine comprises modified nucleic acids, such as a recombinant mRNA encoding the protein of interest, and including one, two, or more than two different nucleoside modifications. In some aspects, the modified nucleic acid exhibits reduced degradation in a cell into which the nucleic acid is introduced, relative to a corresponding unmodified nucleic acid. For example, the degradation rate of the modified nucleic acid is reduced by about 10%, 20%, 30%, 40%, 50%, 60%, 70%, 80%, 90%, or greater than 90%, compared to the degradation rate of the corresponding unmodified nucleic acid.

In some aspects, modified nucleosides include pyridin-4-one ribonucleoside, 5-aza-uridine, 2-thio-5-aza-uridine, 2-thiomidine, 4-thio-pseudomidine, 2-thio-pseudowidine, 5-hydroxyuridine, 3-methylmidine, 5-carboxymethyl-uridine, 1-carboxymethyl-pseudoutidine, 5-propynyl-uridine, 1-propynyl-pseudomidine, 5-taurinomethyluridine, 1-taurinomethyl-pseudouridine, 5-taw.inomethyl-2-thio-utidine, 1-taurinomethyl-4-thio-uridine, 5-methyl-uridine, 1-methyl-pseudouridine, 4-thio-1-methyl-pseudouridine, 2-thio-1-methyl-pseudoutidine, 1-methyl-1-deaza-pseudomidine, 2-thio-1-methyl-1-deaza-pseudouridine, dihydrouridine, dihydropseudouridine, 2-thio-dihydromidine, 2-thio-dihydropseudoulidine, 2-methoxyuridine, 2-methoxy-4-thio-uridine, 4-methoxy-pseudomidine, and 4-methoxy-2-thio-pseudouridine.

In some aspects, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebulruine, 5-methyl-zebularine, 5-aza-2-thio-zebulru.ine, 2-thio-zebulaiine, 2-methoxy-cytidine, 2-methoxy-5-methyl-cytidine, 4-methoxy-pseudoisocytidine, and 4-methoxy-1-methyl-pseudoisocytidine.

In other aspects, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In specific aspects, a modified nucleoside is 5′-O-(1-Thiophosphate)-Adenosine, 5′-O-(1-Thiophosphate)-Cytidine, 5′-O-(1-thiophosphate)-Guanosine, 5′-O-(1-Thiophophate)-Uridine or 5′-O-(1-Thiophosphate)-Pseudouridine.

As disclosed in U.S. Pat. No. 9,701,965, incorporated herein by reference, the α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment. Phosphorothioate linked nucleic acids are expected to also reduce the innate immune response through weaker binding/activation of cellular innate immune molecules.

In other aspects, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, J-methyl-6-thio-guanosine, N2-methyl-6-thio-guanosine, and N2,N2-dimethyl-6-thio-guanosine.

The disclosed mRNA can include a modified uridine or 1-methylpseudouridine. mRNA that contain either uridine, or 1-methylpseudouridine in place of uridine, the 1-methylpseudouridine-containing mRNA was translated at a higher level or for a longer duration than the mRNA that contained uridine. Therefore, in some aspects, one or more or all of the uridines contained in the mRNA(s) used in the methods disclosed herein is/are replaced by 1-methylpseudouridine (such as by substituting 1-methylpseudouridine-5′-triphosphate in an IVT reaction to synthesize the RNA in place of uridine-5′-triphosphate). However, in some aspects, the mRNA used in the disclosed methods contains uridine and does not contain 1-methylpseudouridine. In more aspects, the mRNA comprises at least one modified nucleoside (e.g., 1-methylpseudouridine (m1ψ), pseudouridine (ψ), 5-methylcytosine (m⁵C), 5-methyluridine (m⁵U), 2′-O-methyluridine (Um or m^{2′-O}U), 2-thiouridine (s²U), or N6-methyladenosine (m⁶A)) in place of at least a portion of the corresponding unmodified canonical nucleoside (e.g., in place of substantially all of the corresponding unmodified A, C, G, or T canonical nucleoside). In some aspects, the mRNA comprises at least one modified nucleoside wherein the nucleotide is pseudouridine (ψ) or 5-methylcytosine (m⁵C). In some aspects, the mRNA comprises both pseudouridine (ψ) and 5-methylcytosine (m⁵C). In other aspects, the mRNA includes 1-methylpseudouridine.

In addition, in order to accomplish specific goals, a nucleic acid base, sugar moiety, or internucleotide linkage in one or more of the nucleotides of the mRNA that is introduced into a eukaryotic cell in any of the methods disclosed herein can comprise a modified nucleic acid base, sugar moiety, or internucleotide linkage.

Nucleic acids encoding for use in accordance with the disclosure may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

Modified nucleic acids need not be uniformly modified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least about 50% modified nucleotides, at least about 80% modified nucleotides, or at least about 90% modified nucleotides.

When transfected into mammalian cells, the modified mRNA can have a stability of between 12-18 hours or more than 18 hours, such as about 24, 36, 48, 60, 72 or greater than about 72 hours. In some aspects, the modified mRNA is stable for about 12 to about 72 hours, such as about 12 to about 48 hours, about 12 to about 36 hours, or about 12 to about 24 hours.

In a specific non-limiting example, the mRNA component is a modified mRNA with modified uridine, such as a 1-methylpseudouridine in place of uridine and a 7mG(5′)ppp(5′)N1mpNp cap.

Lipid Nanoparticles for Use the mRNA Molecules

In some aspects, an mRNA molecule for use in a vaccine as described herein is encapsulated in a lipid. Lipid Nanoparticles are disclosed, for example, in PCT Publication No. 2021/150891, incorporated herein by reference. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/RNA compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5:505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

In an aspect, an RNA molecule is encapsulated in a nanoparticle. Methods for nanoparticle packaging are well known in the art, and are described, for example, in Bose S, et al (Role of Nucleolin in Human Parainfluenza Virus Type 3 Infection of Human Lung Epithelial Cells. J. Virol. 78:8146. 2004); Dong Y et al. Poly(d,l-lactide-co-glycolide)/montmorillonite nanoparticles for oral delivery of anticancer drugs. Biomaterials 26:6068. 2005); Lobenberg R. et al (Improved body distribution of 14C-labelled AZT bound to nanoparticles in rats determined by radioluminography. J Drug Target 5:171. 1998); Sakuma S R et al (Mucoadhesion of polystyrene nanoparticles having surface hydrophilic polymeric chains in the gastrointestinal tract. Int J Pharm 177:161. 1999); Virovic L et al. Novel delivery methods for treatment of viral hepatitis: an update. Expert Opin Drug Deliv 2:707.2005); and Zimmermann E et al., Electrolyte- and pH-stabilities of aqueous solid lipid nanoparticle (SLN) dispersions in artificial gastrointestinal media. Eur J Pharm Biopharm 52:203.2001). Methods are also disclosed in PCT Publication Nos. WO2021154763, US20210228707, WO2017070626 and US2019/0192646, which are incorporated by reference herein. See, also, Jackson et al., N Engl J Med., 383(20):1920-1921, 2020, incorporated by reference herein.

In some aspects, the mRNA is formulated in a lipid nanoparticle for administration to the subject; for example, comprising a PEG-modified lipid, a non-cationic lipid, a sterol, an ionizable lipid, or any combination thereof. In some aspects, the lipid nanoparticle is composed of 50 mol % ionizable lipid ((2 hydroxyethyl)(6 oxo 6-(undecycloxy)hexyl)amino)octanoate, 10 mol % 1,2 distearoyl sn glycerol-3 phosphocholine (DSPC), 38.5 mol % cholesterol, and 1.5 mol % 1-monomethoxypolyethyleneglycol-2,3,dimyristylglycerol with polyethylene glycol of average molecular weight 2000 (PEG2000 DMG). The mRNA/lipid nanoparticle composition may be provided in any suitable carrier, such as a sterile liquid for injection at a concentration of 0.5 mg/mL in 20 mM trometamol (Tris) buffer containing 87 mg/mL sucrose and 10.7 mM sodium acetate, at pH 7.5 and with appropriate diluent.

### Pharmaceutical Compositions and Administration

A MBV, as described herein, can be formulated with a vaccine, and optionally an adjuvant and/or cytokine, such as IL-12. These components can be formulated with pharmaceutically acceptable carriers to help retain biological activity while also promoting increased stability during storage within an acceptable temperature range. Potential carriers include, but are not limited to, physiologically balanced culture medium, phosphate buffer saline solution, water, emulsions (e.g., oil/water or water/oil emulsions), various types of wetting agents, cryoprotective additives or stabilizers such as proteins, peptides or hydrolysates (e.g., albumin, gelatin), sugars (e.g., sucrose, lactose, sorbitol), amino acids (e.g., sodium glutamate), or other protective agents. The resulting aqueous solutions may be packaged for use as is or lyophilized. Lyophilized preparations are combined with a sterile solution prior to administration for either single or multiple dosing.

In some aspects, provided herein are pharmaceutical compositions (e.g., vaccine compositions). For example, in some aspects, provided pharmaceutical compositions further comprise a pharmaceutically acceptable carrier.

In some aspects, pharmaceutical compositions disclosed herein further comprise an antibiotic.

Pharmaceutical compositions disclosed herein may be specially formulated for administration in solid or liquid form, including those adapted for the following: (1) oral administration, for example, drenches (aqueous or non-aqueous solutions or suspensions), tablets (e.g., those targeted for buccal, sublingual, or systemic absorption), boluses, powders, granules, or pastes (e.g., for application to the tongue); (2) parenteral administration, for example, by subcutaneous, intramuscular, intravenous, or epidural injection. Non-limiting examples of formulations suitable for parenteral administration include, without limitation, sterile solutions, sterile suspensions, and sustained-release formulations; or (3) intranasal administration, such as a mist or spray.

In general, formulations may be prepared by uniformly and intimately bringing into association one or more composition components described herein with liquid pharmaceutically acceptable carriers, finely divided solid pharmaceutically acceptable carriers, or both, and then, if necessary, shaping the product.

Pharmaceutical compositions suitable for parenteral administration may be provided as pharmaceutically acceptable sterile isotonic aqueous or nonaqueous solutions, dispersions, suspensions or emulsions. Alternatively or additionally, pharmaceutical compositions for parenteral administration may be provided as sterile powders, which may be reconstituted into sterile injectable solutions or dispersions just prior to use. Such injectable solutions may contain one or more agents that render the formulation isotonic with the blood of the intended recipient, one or more suspending agents, and/or one or more thickening agents. For example, injectable solutions may comprise one or more of sugars, alcohols, antioxidants, buffers, bacteriostats, and solutes.

Examples of suitable aqueous and nonaqueous pharmaceutically acceptable carriers include, but are not limited to, water, ethanol, polyols (such as glycerol, propylene glycol, polyethylene glycol, and the like), and suitable mixtures thereof; vegetable oils, such as olive oil; and injectable organic esters, such as ethyl oleate. Proper fluidity can be maintained, for example, by the use of coating materials (such as lecithin), by the maintenance of the required particle size in the case of dispersions, and by the use of surfactants.

Pharmaceutical compositions provided herein may be formulated as emulsions. For examples, provided are vaccine compositions formulated as emulsions, which provide an alternative to aluminum-based vaccines. Emulsion formulations may be prepared by emulsifying antigens dissolved in an aqueous buffer with an oil, such as any metabolizable oil, as further described herein. Emulsion formulations may form a short-lived depot to facilitate vaccine phagocytosis by innate immune cells, which results in an immune response (Leenaars, Koedam et al. 1998). The oils used in such emulsions can impart unique immune stimulation and result in stronger immune responses than can vaccines comprising alum adjuvants (De Gregorio, Caproni et al. 2013).

Pharmaceutical compositions disclosed herein may be formulated into pharmaceutically acceptable dosage forms by conventional methods known to those of skill in the art.

In some aspects, an additional physiologically acceptable adjuvant, e.g., in combination with MBV, is employed. Such an additional adjuvant may be used or included in any of a number of ways, including, but not limited to, (i) admixed to other components in a pharmaceutical composition as provided herein after reconstitution of antigens and optional emulsification with a metabolizable oil as defined above, (ii) part of a reconstituted antigen-containing composition as provided herein, (iii) physically linked to antigen(s) to be reconstituted; and (iv) administered separately to the subject. The additional adjuvant can, for example, slow release of antigen (e.g., the additional adjuvant can be a liposome) and/or it can be an adjuvant that is immunogenic in its own right, thereby functioning synergistically with antigens (i.e., antigens present in a provided composition). In some embodiments, the adjuvant is a cytokine, such as, but not limited to, IL-12.

For example and without limitation, the additional adjuvant can be a known adjuvant or other substance that promotes antigen uptake, recruits immune system cells to the site of administration, and/or facilitates the immune activation of responding lymphoid cells. Examples of suitable additional adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some aspects, the additional adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, MONTANIDE™, N-acetyl-muramyl-L-alanyl-D-isoglutamine, Pam3CSK4, quil A, trehalose dimycolate, or zymosan. In some aspects, the additional adjuvant induces a mixed type 1/type 17 immune response. In certain aspects, the pharmaceutical composition may optionally include an adjuvant to enhance an immune response of the host. Suitable adjuvants for such use are, for example, toll-like receptor agonists, alum, AlPO4, alhydrogel, Lipid-A and derivatives or variants thereof, oil-emulsions, saponins, neutral liposomes, liposomes containing the vaccine and cytokines, non-ionic block copolymers, and chemokines. Non-ionic block polymers containing polyoxyethylene (POE) and polyxylpropylene (POP), such as POE-POP-POE block copolymers, MPL™ (3-O-deacylated monophosphoryl lipid A; Corixa, Hamilton, IN) and IL-12 (Genetics Institute, Cambridge, MA), may be used as an adjuvant (Newman et al., 1998, Critical Reviews in Therapeutic Drug Carrier Systems 15:89-142). These adjuvants have the advantage in that they help to stimulate the immune system in a non-specific way, thus enhancing the immune response to a pharmaceutical product.

Formulated compositions, especially liquid formulations, may contain a bacteriostat to prevent or minimize degradation during storage, including but not limited to effective concentrations (usually ≤1% w/v) of benzyl alcohol, phenol, m-cresol, chlorobutanol, methylparaben, and/or propylparaben. A bacteriostat may be contraindicated for some patients; therefore, a lyophilized formulation may be reconstituted in a solution either containing or not containing such a component.

In some aspects, a composition comprising the effective amount of the vaccine and the effective amount MBV is administered to the subject. In some aspects, the vaccine and the MBV are each administered separately to the subject. In some aspects, the MBV are administered to the subject immediately before or immediately after the vaccine is administered. In certain aspects, the MBV are administered to the subject within 1 minutes, 2 minutes, 3 minutes, 4 minutes or 5 minutes of the vaccine being administered.

In certain aspects, the vaccine and the MBV are administered to the subject on a first date and the subject subsequently receives one or more further administrations of an effective amount of MBV without further administration of vaccine. In certain aspects, the subject receives a further administration of MBV 1 week, 2 weeks, 3 weeks, 4 weeks, 5 weeks, 6 weeks, 7 weeks, 8 weeks, 3 months, 4, months, 5 months, and/or 6 months after the first date.

According to some aspects, a subject is administered 1×10⁶ to 1×10¹² MBV per kg of body weight per administration. In another aspect, a subject is administered 1×10⁷ to 1×10¹¹ MBV per kg of body weight per administration. In another aspect, a subject is administered 1×10⁷ to 1×10⁸ MBV per kg of body weight per administration. In another aspect, a subject is administered 1×10⁸ to 1×10¹⁰ MBV per kg of body weight per administration. In another aspect, a subject is administered 1×10⁹ to 1×10¹⁰ MBV per kg of body weight per administration. In another aspect, a subject is administered 1×10⁶ to 1×10⁸ MBV per kg of body weight per administration. In another aspect, a subject is administered 1×10⁷ to 1×10⁹ MBV per kg of body weight per administration. In another aspect, a subject is administered 1×10⁸ to 1×10¹¹ MBV per kg of body weight per administration. In another aspect, a subject is administered 1×10⁹ to 1×10¹¹ MBV per kg of body weight per administration. In another aspect, a subject is administered 1×10¹⁰ to 1×10¹¹ MBV per kg of body weight per administration. In another aspect, a subject is administered 1×10¹¹ to 1×10¹² MBV per kg of body weight per administration. In another aspect, a subject is administered 1×10⁶ to 1×10¹⁴ MBV per kg of body weight per administration. In another aspect, a subject is administered 1×10¹² to 1×10¹⁴ MBV per kg of body weight per administration. In one aspect, administration of MBV according to any of the aforementioned amounts is by systemic administration. In one aspect, administration of MBV according to any of the aforementioned amounts is by intramuscular administration. In one aspect, administration of MBV according to any of the aforementioned amounts is by nasal administration. In one aspect, administration of MBV according to any of the aforementioned amounts is by oral administration.

In another aspect, the MBV are administered in an amount 1×10⁷ to 1×10¹¹ MBV per dose in a set volume of liquid, for example physiologic saline. For example, in one aspect, the volume is 50 μL-500 μL. For example, in one aspect the volume is 100 μL-300 μL. For example, in one aspect the volume is 200 μL-400 μL. For example, in one aspect the volume is 300 μL-400 μL. In a further aspect, the volume is 250 μL. In a further aspect, the volume is 300 μL. For example, in one aspect, the number of MBV in the dose is 1×10⁸ to 1×10¹⁰ MBV. For example, in one aspect, the number of MBV in the dose is 1×10⁹ MBV.

In one aspect, the MBV are administered with the vaccine on the same day, e.g., a first date, as an initial administration. For example, the vaccine is administered followed immediately by administration of the MBV. For example, the MBV are administered followed immediately by administration of the vaccine. For example, the vaccine and the MBV are administered within one minute of one another, within two minutes of one another, within three minutes of one another, within four minutes of one another, or within 5 minute of one another. In one aspect, the MBV are formulated as part of a composition that includes the vaccine antigen and therefore are administered simultaneously with the vaccine antigen.

The MBV may be administered more than once as part of a protocol to boost the immune response of the subject to the vaccine. For example, on a first date, a subject is administered the vaccine and the MBV, either as separate administrations, or as a composition containing both the MBV and the vaccine, as an initial administration. Thereafter, the subject may receive one or more subsequent administrations of MBV on one or more different dates. For example, the subject receives an administration of an effective amount of MBV one week after the first date of administration, two weeks after the first date of administration, three weeks after the first date of administration, four weeks after the first date of administration, five weeks after the first date of administration, six weeks after the first date of administration, seven weeks after the first date of administration, eight weeks after the first date administration, 12 weeks after the first date of administration, 1 month after the first date of administration, 2 months after the first date of administration, 3 months after the first date of administration, 4 months after the first date of administration, 6 months after the first date of administration, 8 months after the first date of administration, 9 months after the first date of administration or 1 year after the first date of administration.

For example, the subject receives one or more administrations of an effective amount of MBV one week after the first date of administration, two weeks after the first date of administration, three weeks after the first date of administration, four weeks after the first date of administration, five weeks after the first date of administration, six weeks after the first date of administration, seven weeks after the first date of administration, eight weeks after the first date of administration, 12 weeks after the first date of administration, 1 month after the first date of administration, 2 months after the first date of administration, 3 months after the first date of administration, 4 months after the first date of administration, 6 months after the first date of administration, 8 months after the first date of administration, 9 months after the first date of administration and/or 1 year after the first date of administration. In one aspect, the subject receives a subsequent MBV administration 1 week, 2 weeks, 3 weeks, 4 weeks and 5 weeks after the first date of vaccination and MBV administration. In one aspect, the subject receives a subsequent MBV administration one month after the first date of vaccination and MBV administration. In one aspect, the subject receives a subsequent MBV administration four weeks after the first date of vaccination and MBV administration. In one aspect, the subject receives a subsequent MBV administration two weeks after the initial vaccination and MBV administration.

Pharmaceutical compositions comprising MBV as described herein as an active ingredient will normally be formulated with an appropriate solid or liquid carrier, depending upon the particular mode of administration chosen. For example, pharmaceutically and physiologically acceptable fluid vehicles such as water, physiological saline, other balanced salt solutions, aqueous dextrose, glycerol or the like can be used for injectable formulations, or for nebulized or aerosolized formulations. Excipients that can be included are, for instance, proteins, such as human serum albumin or plasma preparations. If desired, the pharmaceutical composition to be administered may also contain minor amounts of non-toxic auxiliary substances, such as wetting or emulsifying agents, preservatives, and pH buffering agents and the like, for example sodium acetate or sorbitan monolaurate. Actual methods of preparing such dosage forms are known, or will be apparent, to those skilled in the art.

The pharmaceutical compositions that comprise MBV, in some aspects, will be formulated in unit dosage form, suitable for individual administration of precise dosages. The amount of active compound(s) administered will be dependent on the subject being treated, the severity of the disease, and the manner of administration, and is best left to the judgment of the prescribing clinician. Within these bounds, the formulation to be administered will contain a quantity of the active component(s) in amounts effective to achieve the desired effect in the subject being treated.

Dosage treatment may be a single dose schedule or a multiple dose schedule to ultimately deliver 1×10⁶ to 1×10¹² MBV (i.e., an absolute number of vesicles) per kg body weight per administration. Administration may be provided as a single administration, a periodic bolus or as continuous infusion, such as by continuous release for a specific period from a sustained-release drug or drug delivery device. The subject may be administered as many doses as appropriate. If multiple doses are administered, administration can be intermittent. In example aspects, administration (such as systemic administration, for example, intravenous administration, or any other route of administration) of a therapeutically effective amount of MBV can be performed once, or can be performed repeatedly, such as 1, 2, 3, 4, 5, 6, 7, 8, 9, or 10 times.

Individual doses of MBV are typically not less than an amount required to produce a measurable effect on the subject, and may be determined based on the pharmacokinetics and pharmacology for absorption, distribution, metabolism, and excretion (“ADME”) of the subject composition or its by-products and, thus, based on the disposition of the composition within the subject. This includes consideration of the route of administration as well as dosage amount, which can be adjusted for local and systemic (for example, intravenous) applications. Effective amounts of dose and/or dose regimen can readily be determined empirically from preclinical assays, from safety and escalation and dose range trials, individual clinician-patient relationships, as well as in vitro and in vivo assays.

A therapeutically effective amount of MBV can be suspended in a pharmaceutically acceptable carrier (such as in a pharmaceutical preparation), for example, in an isotonic buffer solution at a pH of about 3.0 to about 8.0, preferably at a pH of about 3.5 to about 7.4, 3.5 to 6.0, or 3.5 to about 5.0. Useful buffers include sodium citrate-citric acid and sodium phosphate-phosphoric acid, and sodium acetate/acetic acid buffers. Other agents can be added to the compositions, such as preservatives and anti-bacterial agents. These compositions can be administered locally or systemically, such as intravenously.

The pharmaceutical compositions of the disclosure can contain as pharmaceutically acceptable vehicles substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

In some aspects, the composition can be provided as a sterile composition. The pharmaceutical composition typically contains an effective amount of a disclosed immunogen and can be prepared by conventional techniques. Typically, the amount of immunogen in each dose of the immunogenic composition is selected as an amount which elicits an immune response without significant, adverse side effects. In some aspects, the composition can be provided in unit dosage form for use to elicit an immune response in a subject, for example, to prevent infection in the subject. A unit dosage form contains a suitable single preselected dosage for administration to a subject, or suitable marked or measured multiples of two or more preselected unit dosages, and/or a metering mechanism for administering the unit dose or multiples thereof. In other aspects, the composition further includes an adjuvant.

### Methods of Inducing an Immune Response

The disclosed vaccine antigens, polynucleotides and vectors encoding the disclosed vaccine antigens, and compositions including same, can be used, in conjunction with an effective amount MBV, in methods of inducing an immune response to any pathogen. The disclosed vaccine antigens, polynucleotides and vectors encoding the disclosed vaccine antigens, and compositions including same, can be used, in conjunction with an effective amount MBV, in methods of inducing an immune response to any tumor. In some aspects, such compositions comprise the vaccine and the MBV. In other aspects, the vaccine and the MBV are administered in separate compositions. In some embodiments, an adjuvant, such as a cytokine, can be included with the vaccine, the MBV, or with both.

In aspects, the immune response can include a humoral immune response, a cell-mediated immune response, or both. In some aspects, an immune response induces myeloid cells. In some aspects, an immune response induces IgM and/or IgG antibodies. In some aspects, an immune response induces production of IFNγ and/or IL-23.

A humoral response can be determined, for example, by a standard immunoassay for antibody levels in a serum sample from the subject receiving the pharmaceutical composition.

A cellular immune response is a response that typically involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of a pharmaceutically acceptable composition. For example, after incubation of PBMCs with a stimulator for an appropriate period, [³H]thymidine incorporation can be determined. The percentage of proliferating T cells can be determined using flow cytometry. Another way to measure cellular immunity involves measuring circulating frequencies of T cells secreting proinflammatory Type-1 and/or Type-17 cytokines in response to the antigen.

To identify subjects for prophylaxis or treatment according to the methods of the disclosure, accepted screening methods are employed to determine risk factors associated with a targeted or suspected disease or condition, or to determine the status of an existing disease or condition in a subject. These screening methods include, for example, conventional work-ups to determine environmental, familial, occupational, and other such risk factors that may be associated with the targeted or suspected disease or condition, as well as diagnostic methods, such as various ELISA and other immunoassay methods to detect and/or characterize an infection. These and other routine methods allow the clinician to select patients in need of therapy using the methods and pharmaceutical compositions of the disclosure. In accordance with these methods and principles, a composition can be administered according to the teachings herein, or other conventional methods, as an independent prophylaxis or treatment program, or as a follow-up, adjunct or coordinate treatment regimen to other treatments.

Immunogenic composition that include a vaccine and/or MBV, as disclosed herein, can be used in coordinate (or prime-boost) immunization protocols or combinatorial formulations. In certain aspects, novel combinatorial immunogenic compositions and coordinate immunization protocols employ separate immunogens or formulations, each directed toward eliciting an immune response. Separate immunogenic compositions that elicit the immune response can be combined in a polyvalent immunogenic composition administered to a subject in a single immunization step, or they can be administered separately (in monovalent immunogenic compositions) in a coordinate immunization protocol.

In one aspect, a suitable immunization regimen includes at least two separate inoculations with one or more immunogenic compositions including a disclosed vaccine and/or MBV, with a second inoculation being administered more than about two, about three to eight, or about four, weeks following the first inoculation. A third inoculation can be administered several months after the second inoculation, and in specific aspects, more than about five months after the first inoculation, more than about six months to about two years after the first inoculation, or about eight months to about one year after the first inoculation. Periodic inoculations beyond the third are also desirable to enhance the subject's “immune memory.” The adequacy of the vaccination parameters chosen, e.g., formulation, dose, regimen and the like, can be determined by taking aliquots of serum from the subject and assaying antibody titers during the course of the immunization program. Alternatively, the T cell populations can be monitored by conventional methods. In addition, the clinical condition of the subject can be monitored for the desired effect, e.g., prevention of infection or progression to disease, improvement in disease state (e.g., reduction in viral load), or reduction in transmission frequency to an uninfected partner. If such monitoring indicates that vaccination is sub-optimal, the subject can be boosted with an additional dose of immunogenic composition, and the vaccination parameters can be modified in a fashion expected to potentiate the immune response. Thus, for example, a dose of a disclosed vaccine and/or MBV can be increased or the route of administration can be changed.

It is contemplated that there can be several boosts, and that each boost can be a different immunogen. It is also contemplated in some examples that the boost may be the same immunogen as another boost, or the prime.

The prime and the boost can be administered as a single dose or multiple doses, for example, two doses, three doses, four doses, five doses, six doses or more can be administered to a subject over days, weeks or months. Multiple boosts can also be given, such one to five, or more. Different dosages can be used in a series of sequential inoculations. For example, a relatively large dose in a primary inoculation and then a boost with relatively smaller doses. The immune response against the selected antigenic surface can be elicited by one or more inoculations of a subject. The boost can include both the vaccine and MBV. The boost can include MBV only. The boost can include the vaccine only.

In several aspects, a disclosed immunogenic composition can be administered to the subject simultaneously with the administration of an adjuvant. In other aspects, the immunogenic composition can be administered to the subject after the administration of an adjuvant and within a sufficient amount of time to elicit the immune response. In certain aspects, the adjuvant comprises MBV. In certain aspects, the adjuvant comprises MBV in combination with one or more additional adjuvants described herein.

Determination of effective dosages in this context is typically based on animal model studies followed up by human clinical trials and is guided by administration protocols that significantly reduce the occurrence or severity of targeted disease symptoms or conditions in the subject, or that elicit a desired response in the subject (such as a neutralizing immune response). Suitable models in this regard include, for example, murine, rat, porcine, feline, ferret, non-human primate, and other accepted animal model subjects known in the art. Alternatively, effective dosages can be determined using in vitro models (for example, immunologic and histopathologic assays). Using such models, only ordinary calculations and adjustments are required to determine an appropriate concentration and dose to administer an effective amount of the composition (for example, amounts that are effective to elicit a desired immune response or alleviate one or more symptoms of a targeted disease). In alternative aspects, an effective amount or effective dose of the composition may simply inhibit or enhance one or more selected biological activities correlated with a disease or condition, as set forth herein, for either therapeutic or diagnostic purposes.

Dosage can be varied by the attending clinician to maintain a desired concentration at a target site (for example, systemic circulation). Higher or lower concentrations can be selected based on the mode of delivery, for example, trans-epidermal, rectal, oral, pulmonary, or intranasal delivery versus intravenous or subcutaneous delivery. The actual dosage of disclosed immunogen will vary according to factors such as the disease indication and particular status of the subject (for example, the subject's age, size, fitness, extent of symptoms, susceptibility factors, and the like), time and route of administration, other drugs or treatments being administered concurrently, as well as the specific pharmacology of the composition for eliciting the desired activity or biological response in the subject. Dosage regimens can be adjusted to provide an optimum prophylactic or therapeutic response.

A non-limiting range for an effective amount of an vaccine, such as a vaccine antigen, within the methods and immunogenic compositions of the disclosure is about 0.0001 mg/kg body weight to about 10 mg/kg body weight, such as about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.5 mg/kg, about 2 mg/kg, about 2.5 mg/kg, about 3 mg/kg, about 4 mg/kg, about 5 mg/kg, or about 10 mg/kg, for example, 0.01 mg/kg to about 1 mg/kg body weight, about 0.05 mg/kg to about 5 mg/kg body weight, about 0.2 mg/kg to about 2 mg/kg body weight, or about 1.0 mg/kg to about 10 mg/kg body weight. In some aspects, the dosage includes a set amount of a disclosed immunogen such as from about 1-300 μg, for example, a dosage of about 1, 2, 3, 4, 5, 6, 7, 8, 9, 10, 15, 20, 30, 40, 50, 60, 70, 80, 90, 100, 150, 200, 250, or about 300 μg.

The dosage and number of doses will depend on the setting, for example, in an adult or anyone primed by prior infection or immunization, a single dose may be a sufficient booster. In naïve subjects, in some examples, at least two doses would be given, for example, at least three doses. In some aspects, an annual boost is given, for example, along with an annual influenza vaccination.

An infection does not need to be completely inhibited for the methods to be effective. For example, elicitation of an immune response to a pathogen can reduce or inhibit infection with the pathogen by a desired amount, for example, by at least 10%, at least 20%, at least 50%, at least 60%, at least 70%, at least 80%, at least 90%, at least 95%, at least 98%, or even at least 100% (elimination or prevention of detectable infected cells), as compared to infection in the absence of the therapeutic agent. In additional examples, viral replication can be reduced or inhibited by the disclosed methods. Viral replication does not need to be completely eliminated for the method to be effective. For example, the immune response elicited using one or more of the disclosed immunogens can reduce viral replication by a desired amount, for example, by at least 10%, at least 20%, at least 50%, at least 60%, at least 70%, at least 80%, at least 90%, at least 95%, at least 98%, or even at least 100% (elimination or prevention of detectable virus replication), as compared to viral replication in the absence of the immune response.

Following immunization of a subject, serum can be collected from the subject at appropriate time points, frozen, and stored for neutralization testing. Methods to assay for neutralization activity, and include, but are not limited to, plaque reduction neutralization (PRNT) assays, microneutralization assays, flow cytometry based assays, single-cycle infection assays (e.g., as described in Martin et al. (2003) Nature Biotechnology 21:71-76), and pseudovirus neutralization assays (e.g., as described in Georgiev et al. (Science, 340, 751-756, 2013), Seaman et al. (J. Virol., 84, 1439-1452, 2005), and Mascola et al. (J. Virol., 79, 10103-10107, 2005), each of which is incorporated by reference herein in its entirety. Macrophage activity can also be assessed. The expression of a cytokine, such as IL-23 or IFNγ can also be assessed. In some aspects, administration of an effective amount of one or more of the disclosed immunogenic compositions to a subject (e.g., by a prime-boost administration) elicits a neutralizing immune response in the subject. Cellular immune responses can also be measured.

One approach to administration of nucleic acids is direct immunization with plasmid DNA, such as with a mammalian expression plasmid. Immunization by nucleic acid constructs is taught, for example, in U.S. Pat. No. 5,643,578 (which describes methods of immunizing vertebrates by introducing DNA encoding a desired antigen to elicit a cell-mediated or a humoral response), and U.S. Pat. Nos. 5,593,972 and 5,817,637 (which describe operably linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids (or of the synthetic peptides themselves), and immune-stimulating constructs, or ISCOMS™, negatively charged cage-like structures of 30-40 nm in size formed spontaneously on mixing cholesterol and Quil ATM (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990). This administration can be combined with the administration of MBV.

In some aspects, a plasmid DNA vaccine is used to express a disclosed immunogen in a subject. For example, a nucleic acid molecule encoding a disclosed immunogen can be administered to a subject to elicit an immune response. In some aspects, the nucleic acid molecule can be included on a plasmid vector for DNA immunization, such as the pVRC8400 vector (described in Barouch et al., J. Virol, 79, 8828-8834, 2005, which is incorporated by reference herein). This administration can be combined with the administration of MBV.

In another approach to using nucleic acids for immunization, a disclosed immunogen can be expressed by attenuated viral hosts or vectors or bacterial vectors. Recombinant vaccinia virus, adeno-associated virus (AAV), herpes virus, retrovirus, cytomegalovirus or other viral vectors can be used to express the peptide or protein, thereby eliciting a CTL response. For example, vaccinia vectors and methods useful in immunization protocols are described in U.S. Pat. No. 4,722,848. BCG (Bacillus Calmette Guerin) provides another vector for expression of the peptides (see Stover, Nature 351:456-460, 1991). This administration can be combined with the administration of MBV.

In one aspect, a nucleic acid is introduced directly into cells. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 μg/kg to about 50 mg/kg, and typically are about 0.005 mg/kg to about 5 mg/kg (see, e.g., U.S. Pat. No. 5,589,466). This administration can be combined with the administration of MBV.

In some aspects, an mRNA is utilized. Various aspects of dosage ranges of mRNA can be used in methods of the present disclosure. In one aspect, the dosage is in the range of about 0.1 to about 0.9 μg/day. In some aspects, the dosage range can be about 0.1 to about 0.8 μg/day, about 0.1 to about 0.7 μg/day, about 0.1 to about 0.6 μg/day, about 0.1 to about 0.5 μg/day, about 0.1 to about 0.4 μg/day, about 0.1 to about 0.3 μg/day, or about 0.1 to about 0.2 μg/day. In more aspects, the dosage range can be about 0.2 to about 0.9 μg/day, about 0.3 to about 0.9 μg/day, about 0.4 to about 0.9 μg/day, about 0.5 to about 0.9 μg/day, about 0.6 to about 0.9 μg/day, about 0.7 to about 0.9 μg/day, or about 0.8 to about 0.9 μg/day. The dose can be about 0.1 μg/day, about 0.2 μg/day, about 0.3 μg/day, about 0.4 μg/day, about 0.5 μg/day, about 0.6 μg/day, about 0.7 μg/day, about 0.8 μg/day or about 0.9 μg/day.

In one aspect, the dosage is in the range of 1-10 μg/day. In another aspect, the dosage is 2-10 μg/day. In another aspect, the dosage is 3-10 μg/day. In another aspect, the dosage is 5-10 μg/day. In another aspect, the dosage is 2-20 μg/day. In another aspect, the dosage is 3-20 μg/day. In another aspect, the dosage is 5-20 μg/day. In another aspect, the dosage is 10-20 μg/day. In another aspect, the dosage is 3-40 μg/day. In another aspect, the dosage is 5-40 μg/day. In another aspect, the dosage is 10-40 μg/day. In another aspect, the dosage is 20-40 μg/day. In another aspect, the dosage is 5-50 μg/day. In another aspect, the dosage is 10-50 μg/day. In another aspect, the dosage is 20-50 μg/day. In one aspect, the dosage is 1-100 μg/day. In another aspect, the dosage is 2-100 μg/day. In another aspect, the dosage is 3-100 μg/day. In another aspect, the dosage is 5-100 μg/day. In another aspect the dosage is 10-100 μg/day. In another aspect the dosage is 20-100 μg/day. In another aspect the dosage is 40-100 μg/day. In another aspect the dosage is 60-100 μg/day.

In another aspect, the dosage is 0.1 μg/day. In another aspect, the dosage is 0.2 μg/day. In another aspect, the dosage is 0.3 μg/day. In another aspect, the dosage is 0.5 μg/day. In another aspect, the dosage is 1 μg/day. In another aspect, the dosage is 2 mg/day. In another aspect, the dosage is 3 μg/day. In another aspect, the dosage is 5 μg/day. In another aspect, the dosage is 10 μg/day. In another aspect, the dosage is 15 μg/day. In another aspect, the dosage is 20 μg/day. In another aspect, the dosage is 30 μg/day. In another aspect, the dosage is 40 μg/day. In another aspect, the dosage is 60 μg/day. In another aspect, the dosage is 80 μg/day. In another aspect, the dosage is 100 μg/day.

In another aspect, the dosage is 10 μg/dose. In another aspect, the dosage is 20 μg/dose. In another aspect, the dosage is 30 μg/dose. In another aspect, the dosage is 40 μg/dose. In another aspect, the dosage is 60 μg/dose. In another aspect, the dosage is 80 μg/dose. In another aspect, the dosage is 100 μg/dose. In another aspect, the dosage is 150 μg/dose. In another aspect, the dosage is 200 μg/dose. In another aspect, the dosage is 300 μg/dose. In another aspect, the dosage is 400 μg/dose. In another aspect, the dosage is 600 μg/dose. In another aspect, the dosage is 800 μg/dose. In another aspect, the dosage is 1000 μg/dose. In another aspect, the dosage is 1.5 mg/dose. In another aspect, the dosage is 2 mg/dose. In another aspect, the dosage is 3 mg/dose. In another aspect, the dosage is 5 mg/dose. In another aspect, the dosage is 10 mg/dose. In another aspect, the dosage is 15 mg/dose. In another aspect, the dosage is 20 mg/dose. In another aspect, the dosage is 30 mg/dose. In another aspect, the dosage is 50 mg/dose. In another aspect, the dosage is 80 mg/dose. In another aspect, the dosage is 100 mg/dose.

In another aspect, the dosage is 10-20 μg/dose. In another aspect, the dosage is 20-30 μg/dose. In another aspect, the dosage is 20-40 μg/dose. In another aspect, the dosage is 30-60 μg/dose. In another aspect, the dosage is 40-80 μg/dose. In another aspect, the dosage is 50-100 μg/dose. In another aspect, the dosage is 50-150 μg/dose. In another aspect, the dosage is 100-200 μg/dose. In another aspect, the dosage is 200-300 μg/dose. In another aspect, the dosage is 300-400 μg/dose. In another aspect, the dosage is 400-600 μg/dose. In another aspect, the dosage is 500-800 μg/dose. In another aspect, the dosage is 800-1000 μg/dose. In another aspect, the dosage is 1000-1500 μg/dose. In another aspect, the dosage is 1500-2000 μg/dose. In another aspect, the dosage is 2-3 mg/dose. In another aspect, the dosage is 2-5 mg/dose. In another aspect, the dosage is 2-10 mg/dose. In another aspect, the dosage is 2-20 mg/dose. In another aspect, the dosage is 2-30 mg/dose. In another aspect, the dosage is 2-50 mg/dose. In another aspect, the dosage is 2-80 mg/dose. In another aspect, the dosage is 2-100 mg/dose. In another aspect, the dosage is 3-10 mg/dose. In another aspect, the dosage is 3-20 mg/dose. In another aspect, the dosage is 3-30 mg/dose. In another aspect, the dosage is 3-50 mg/dose. In another aspect, the dosage is 3-80 mg/dose. In another aspect, the dosage is 3-100 mg/dose. In another aspect, the dosage is 5-10 mg/dose. In another aspect, the dosage is 5-20 mg/dose. In another aspect, the dosage is 5-30 mg/dose. In another aspect, the dosage is 5-50 mg/dose. In another aspect, the dosage is 5-80 mg/dose. In another aspect, the dosage is 5-100 mg/dose. In another aspect, the dosage is 10-20 mg/dose. In another aspect, the dosage is 10-30 mg/dose. In another aspect, the dosage is 10-50 mg/dose. In another aspect, the dosage is 10-80 mg/dose. In another aspect, the dosage is 10-100 mg/dose.

Nucleic acid molecules can be delivered, by microinjection, electroporation, lipid-mediated transfection, peptide-mediated delivery, nanoparticle mediated delivery (such as lipid or polymeric nanoparticle mediate delivery), in association with a degradable polymer, as an mRNA-Lipoplex, as mRNA cargo of PEG-10, or other methods known in the art. The vaccine can include an excipient that confers a protective effect to an mRNA, such that loss of mRNA, as well as transduceability resulting from formulation procedures, packaging, storage, transport, and the like, is minimized. These excipient compositions are therefore considered “nucleic acid-stabilizing” in the sense that they provide higher amounts of in that nucleic acid molecules than their non-protected counterparts, as measured using standard assays, see, for example, Published U.S. Application No. 2012/0219528, incorporated herein by reference. These compositions therefore demonstrate “enhanced transduceability levels” as compared to compositions lacking the particular excipients described herein and are therefore more stable than their non-protected counterparts.

Exemplary excipients that can used to protect from activity degradative conditions include, but are not limited to, detergents, proteins, e.g., ovalbumin and bovine serum albumin, amino acids, e.g., glycine, polyhydric and dihydric alcohols, such as but not limited to polyethylene glycols (PEG) of varying molecular weights, such as PEG-200, PEG-400, PEG-600, PEG-1000, PEG-1450, PEG-3350, PEG-6000, PEG-8000 and any molecular weights in between these values, with molecular weights of 1500 to 6000 preferred, propylene glycols (PG), sugar alcohols, such as a carbohydrate, preferably, sorbitol. The detergent, when present, can be an anionic, a cationic, a zwitterionic or a nonionic detergent. An exemplary detergent is a nonionic detergent. One suitable type of nonionic detergent is a sorbitan ester, e.g., polyoxyethylenesorbitan monolaurate (TWEEN®-20) polyoxyethylenesorbitan monopalmitate (TWEEN®-40), polyoxyethylenesorbitan monostearate (TWEEN®-60), polyoxyethylenesorbitan tristearate (TWEEN®-65), polyoxyethylenesorbitan monooleate (TWEEN®-80), polyoxyethylenesorbitan trioleate (TWEEN®-85), such as TWEEN®-20 and/or TWEEN®-80. These excipients are commercially available from a number of vendors, such as Sigma, St. Louis, Mo.

The amount of the various excipients in any of the disclosed compositions including the mRNA, varies and is readily determined by one of skill in the art. For example, a protein excipient, such as BSA, if present, will can be present at a concentration of between 1.0 weight (wt.) % to about 20 wt. %, such as 10 wt. %. If an amino acid such as glycine is used in the formulations, it can be present at a concentration of about 1 wt. % to about 5 wt. %. A carbohydrate, such as sorbitol, if present, can be present at a concentration of about 0.1 wt % to about 10 wt. %, such as between about 0.5 wt. % to about 15 wt. %, or about 1 wt. % to about 5 wt. %. If polyethylene glycol is present, it can generally be present on the order of about 2 wt. % to about 40 wt. %, such as about 10 wt. % top about 25 wt. %. If propylene glycol is used in the subject formulations, it will typically be present at a concentration of about 2 wt. % to about 60 wt. %, such as about 5 wt. % to about 30 wt. %. If a detergent such as a sorbitan ester (TWEEN®) is present, it can be present at a concentration of about 0.05 wt. % to about 5 wt. %, such as between about 0.1 wt. % and about 1 wt %, see U.S. Published Patent Application No. 2012/0219528, which is incorporated herein by reference. In one example, an aqueous-stabilizing formulation comprises a carbohydrate, such as sorbitol, at a concentration of between 0.1 wt. % to about 10 wt. %, such as between about 1 wt. % to about 5 wt. %. Nucleic acid molecules are generally present in the composition in an amount sufficient to provide a therapeutic effect when given in one or more doses, as defined above.

The mRNA can be included in an inert matrix, optionally with MBV. As one example of an inert matrix, liposomes may be prepared from dipalmitoyl phosphatidylcholine (DPPC), such as egg phosphatidylcholine (PC). Liposomes, including cationic and anionic liposomes, can be made using standard procedures. In a formulation for intrahepatic injection, the liposome capsule degrades due to cellular digestion. Without being bound by theory, these formulations provide the advantages of a slow-release drug delivery system, exposing a subject to a substantially constant concentration of nucleic acid molecule over time. In one example, the nucleic acid molecule can be dissolved in an organic solvent, such as DMSO or alcohol, as previously described, and contain a polyanhydride, poly(glycolic) acid, poly(lactic) acid, or polycaprolactone polymer.

The mRNA and/or MBV may be formulated to permit release over a specific period of time. A release system can include a matrix of a biodegradable material or a material which releases the incorporated nucleic acid molecule by diffusion. The nucleic acid molecule can be homogeneously or heterogeneously distributed within the release system. A variety of release systems may be useful; however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that active ingredients having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other routine modifications), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other routine modifications), copolymers, and mixtures thereof.

Poly(lactide-co-glycolide) microspheres can also be used. Typically the microspheres are composed of a polymer of lactic acid and glycolic acid, which are structured to form hollow spheres. The spheres can be approximately 15-30 microns in diameter and can be loaded with the mRNA and/or MBV.

### EXAMPLES

The disclosure now being generally described, will be more readily understood by reference to the following examples, which are included merely for purposes of illustration of certain aspects and aspects described herein, and is not intended to be limiting.

### Example 1—Matrix Bound Nanovesicles (MBV) have an Effect on the Humoral Response to S. pneumoniae

The effect of matrix bound nanovesicles (MBV) on the capacity for building a humoral response (antibody production), previously has not been assessed. Thus, the effect of MBV on the level of antibody production was evaluated in a suitable animal humoral response model.

### Materials and Methods

### Urinary Bladder Matrix (UBM) MBV Isolation

Porcine urinary bladder matrix (UBM) was decellularized by delamination by manual scrapping and ethanol/peracetic acid incubation. Then, UBM was lyophilized, powdered and digested with LIBERASE™ (Sigma-Aldrich, US) in Tris-HCl buffer overnight at 37° C. and continuous rotation. MBV were isolated then by serial centrifugation and ultracentrifugation. MBV particles concentration per ml were assessed with a NanoSight (Malvern Panalytical, US).

### Animal Humoral Response Model

Immunization and MBV exposure: 6 to 8-week-old, Balb/c mice (Jackson Laboratories, US) were separated into experimental seven groups: (1) Control with no vaccination; (2) Vaccine; (3) Vaccine+1 μg murine IL-12 (BIOLEGEND®, US); (4) Vaccine+1 μg methotrexate (MTX, Sigma-Aldrich, US); (5) Vaccine+1 μg methotrexate (MTX)+1 μg murine IL-12; (6) Vaccine+1×10¹¹ MBV; and (7) Vaccine+1×10¹¹ MBV+1 μg murine IL-12. Animals were administered 1 μg murine IL-12 on days-1, 0, and 1 of the study intraperitoneally (i.p.). Animals were vaccinated i.p. with the human 23-polyvalent S. pneumoniae vaccine (PNEUMOVAX® 23, Merck & Co, US) on day 0 of the study, and MBV and MTX were administered i.p. once weekly through week five. At days 7 and 28, mice were euthanized, serum was collected by blood centrifugation after cardiac puncture (n=3), and titers for IgG and IgM anti-S. pneumoniae serotype 3 capsular polysaccharide antibodies were measured using an enzyme-linked immunosorbent assay (ELISA) assay.

For ELISA, briefly, plates were coated with 100 μg/ml poly-L-lysine (Sigma-Aldrich, US) in PBS at 37° C. for 2 hours, washed with PBS and further coated with serotype 3 pneumococcal polysaccharide (ATCC®, US) overnight at 4° C. Then, plates were washed with PBS and blocked with 3% bovine serum albumin (BSA) for 1 hour at room temperature (RT) under gentle agitation. After blocking, plates were incubated with serial dilutions of mice serum for 2 hours at RT, washed, and incubated with anti-mouse IgG or IgM HRP conjugated antibodies (R&D SYSTEMS®, US and Abcam, US respectively) for 1 hour at RT. Then plates were washed and developed with ELISA substrate (ThermoFisher Scientific) and signals were measured at 450 nm in a plate reader. Positive titers were determined as a signal 2-fold greater than the background signal.

Septic and intranasal infection: 6 to 8-week-old, Balb/c mice (Jackson Laboratories, US) were separated into four groups: (1) Control with no vaccination; (2) Vaccine+1 μg murine IL-12; (3) Vaccine+1 μg MTX+1 μg murine IL-12; (4) Vaccine+1×10¹¹ MBV+1 μg murine IL-12. After immunization, groups receiving MTX or MBV received administration once weekly through week 5 as in the previous experiment. To induce septic infection, mice (n=6 for each group) were infected at week 5 with 10¹¹ CFU of S. pneumoniae (A66.1 strain, Public Health England, UK) in sterile PBS by i.p. injection. For bacterial growth, S. pneumoniae were incubated in Todd Hewitt Broth (THB, Sigma Aldrich, US) with 1% yeast extract (Sigma Aldrich, US) until bacteria reached logarithmic growth. The ratio of CFU to OD was calculated by standard curve between OD and CFU in blood agar plates (ThermoFisher Scientific, US). After infection, animal survival was recorded for 14 days.

For intranasal infection, 1×10⁷ CFU of S. pneumoniae were delivered intranasally at week 5 post-immunization to a second cohort of animals (n=8 for each group). After 10 days, animals were sacrificed, serum was collected by cardiac puncture and titers for IgG and IgM anti-S. pneumoniae serotype 3 capsular polysaccharide antibodies were measured with ELISA as described above. Lungs were collected for histological analysis and the femurs were collected for bone-marrow derived macrophage (BMDM) isolation.

Bone Marrow-Derived Macrophages (BMDM) isolation and ex-vivo challenge: Bone Marrow-Derived Macrophages (BMDM) from the different groups (control; vaccine+1 μg murine IL-12; Vaccine+1 μg MTX+1 μg murine IL-12; vaccine+1×10¹¹ MBV+1 μg murine IL-12) were isolated from the femur bones of sacrificed animals. Briefly, using aseptic techniques, the skin from the proximal hind limb to the tarsus was removed, the coxafemoral joint was disarticulated, and muscle was excised for isolation of the femur. Bones were kept on ice and rinsed in a sterile dish containing macrophage complete medium, which consisted of DMEM (Gibco, US), 10% fetal bovine serum (FBS), 10% L929 fibroblast supernatant, 0.1% beta-mercaptoethanol (Sigma-Aldrich, US), 1% penicillin/streptomycin (PS), 10 mM non-essential amino acids (Gibco, US), and 10 mM Hepes buffer. In a sterile environment, the ends of each bone were transected with sterile scissors and the marrow cavity was flushed with complete macrophage medium using a 30-G needle. Cells were then washed and plated at 10⁶ cell/ml, and allowed to differentiate into macrophages for 7 days at 37° C. and 5% CO₂, changing medium every 48 hours. At day 7, BMDM from each group were treated with either normal medium, LPS at 100 ng/ml or pneumococcal polysaccharide (PPS) at 10 μg/ml for 24 hours at 37° C. and 5% CO₂. The medium was then collected for cytokine analysis and RNA was isolated for gene expression assessment by RT-qPCR.

Cytokines IFNγ, TNFα, IL-6 (Biolegend, US) and IL-23 (R&D Systems, US) were determined according to manufacturers' protocols. RNA was isolated employing TRIZOL® extraction method and measured by RT-qPCR using IL-6, IL-23, IFNγ, MR1, CD74, CSF2 and CXCL2 primers (ThermoFisher, US).

### Results

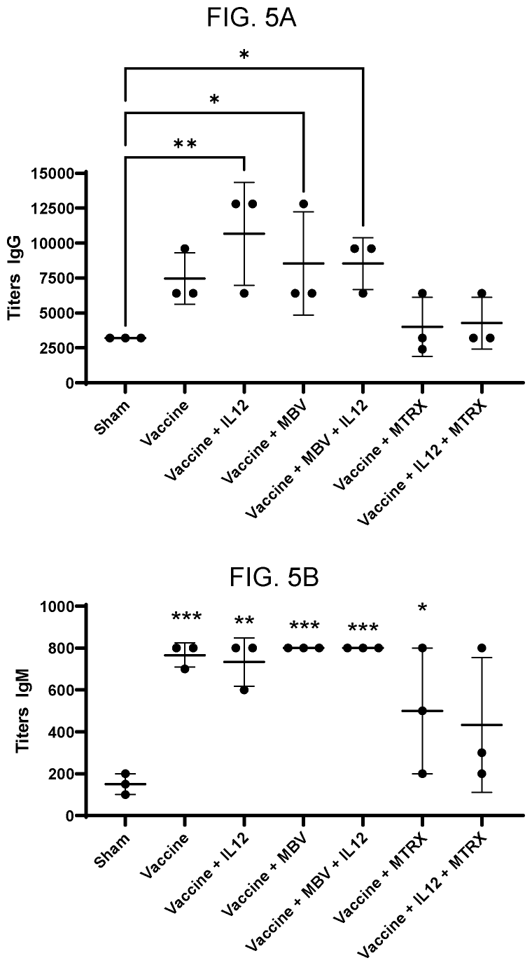

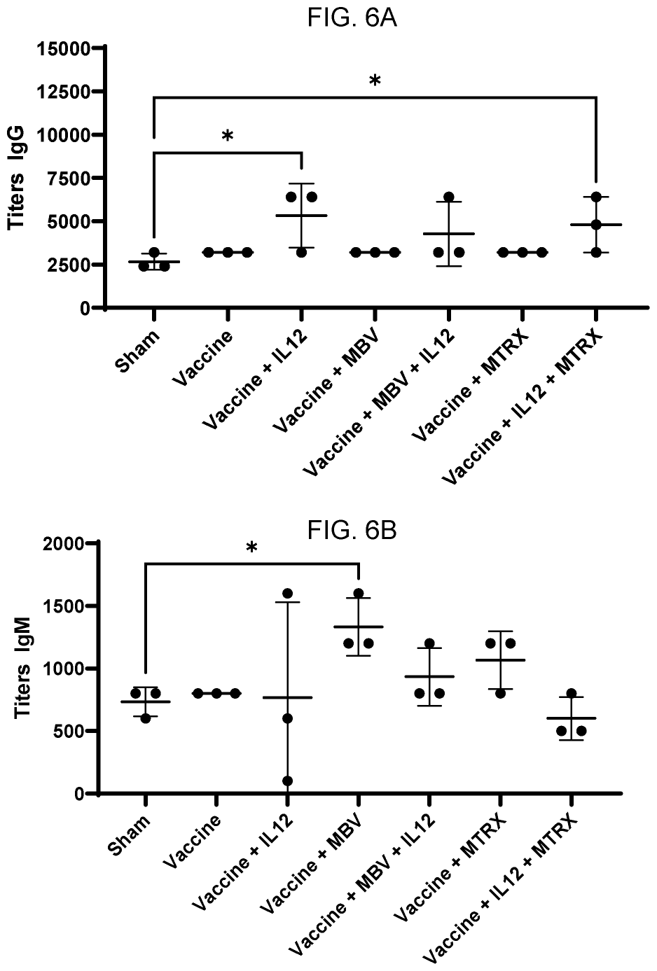

As shown in FIGS. 5A-B (day 7 after vaccine) and FIGS. 6A-B (day 28 after vaccine), IgG and IgM antibody levels were higher in the vaccinated (vaccine, vaccine+IL12) vaccinated+MBV groups (vaccine+MBV, vaccine+MBV+IL12), although there was no statistically significant difference in titer levels between these groups. However, as the vaccine+IL12 and vaccine+MBV+IL12 groups generally presented a more robust response overall, all treatment groups in subsequent experiments involving challenge with S. pneumonia were administered IL-12.

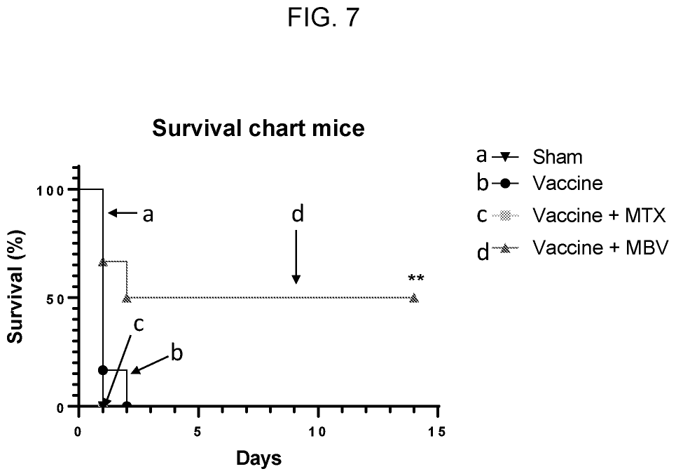

Sepsis infection after immunization for 5 weeks resulted in death of all animals in the sham, vaccine+IL12 and vaccine+MTX+IL12 groups by 2 days after challenge. However, and surprisingly, animals treated with MBV+IL12 had a 50% survival rate 2 weeks post-challenge with S. pneumonia, as shown in FIG. 7.

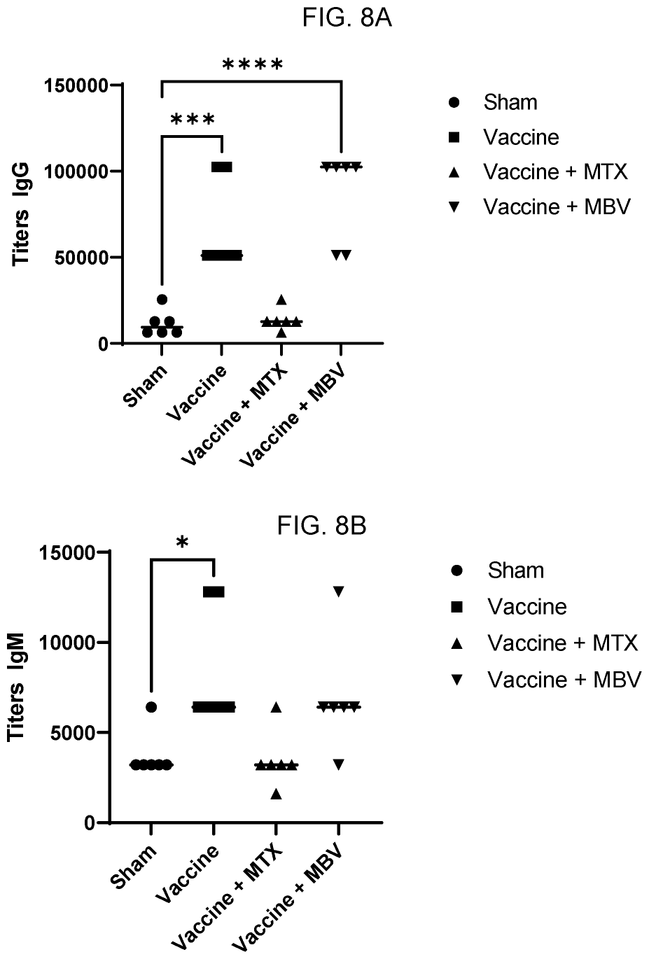

IgM and IgG titer analysis after infection with S. pneumoniae intranasally, as provided in FIGS. 8A-8B, showed that the vaccinated+IL12 and vaccinated+MBV+IL12 mice produced a humoral response that was more robust than the humoral response observed in sham and MTX treated mice. Accordingly, the data demonstrate that administration of MBV did not interfere with the animals' ability to mount a humoral immune response to the vaccine.

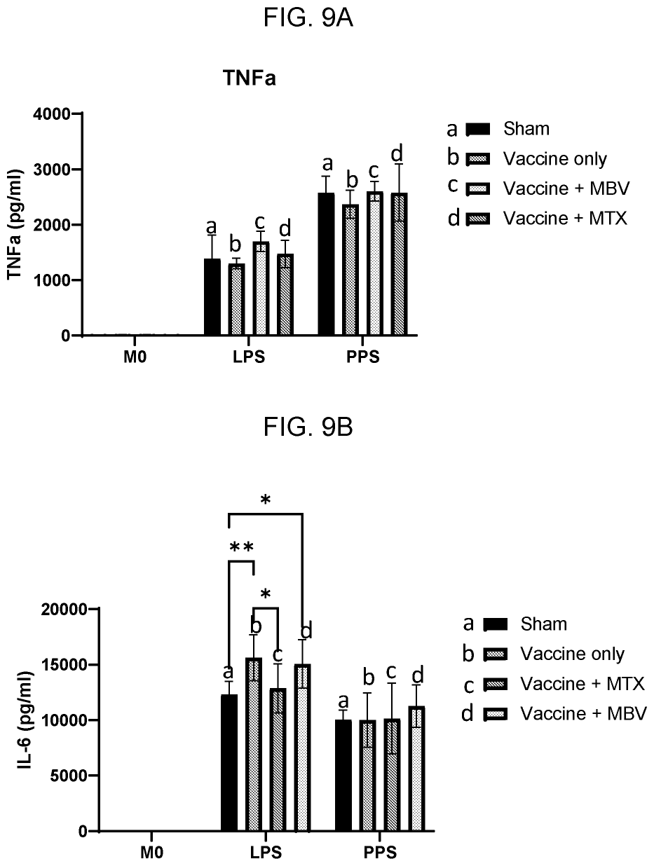

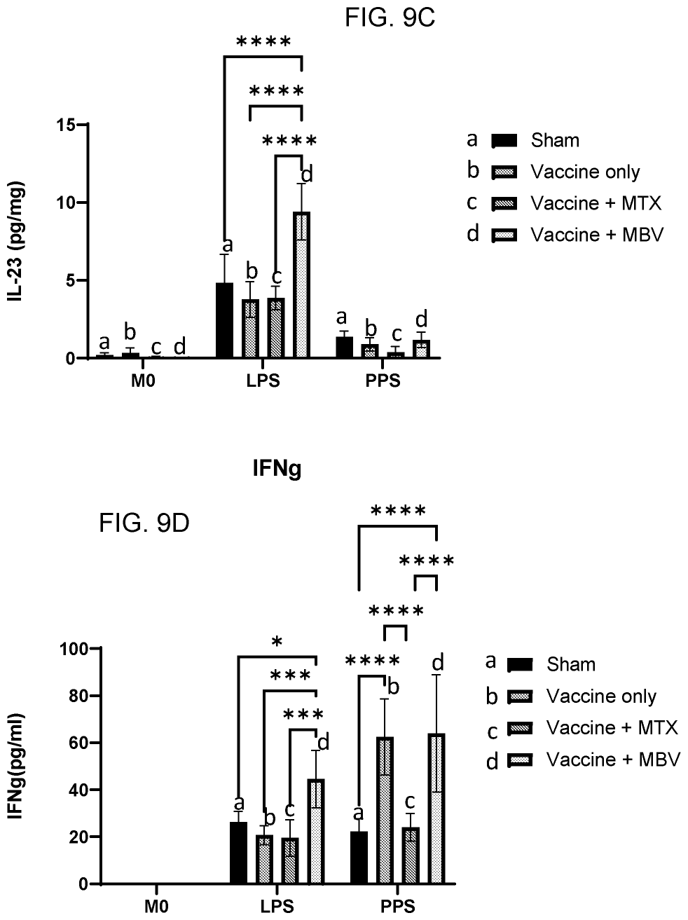

Cytokines that are produced in response to foreign antigens, such as IFNγ and IL23, were found to be higher in MBV+vaccine isolated macrophages when exposed to new microbial antigens (LPS=lipopolysaccharide representative of Gram negative bacterial infection), compared to the other the groups. When macrophages were exposed to previously encountered antigen (PPS=pneumococcal polysaccharide), macrophages from vaccinated and vaccinated+MBV animals had similar responses. The results obtained using PPS show that memory for the specific antigen in the vaccine was not affected by the administration of MBV. The results obtained using LPS surprisingly show that macrophages from mice immunized with both the vaccine and MBV produced increased immune response to other immunogens, indicating that the administration of MBV expanded the response of macrophages to other immunogens. (FIGS. 9A-9D).

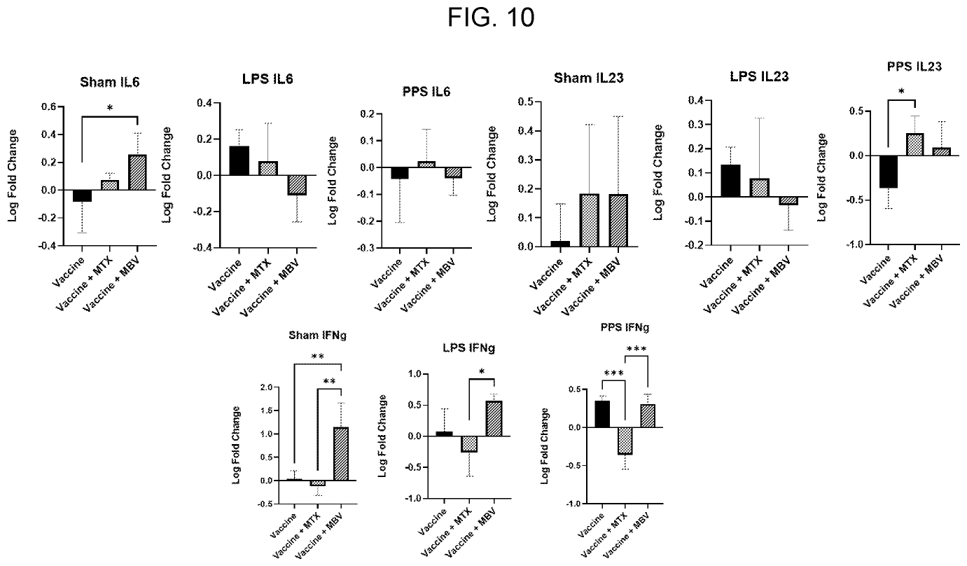

RT-qPCR was used to evaluate expression of cytokines (FIG. 10).

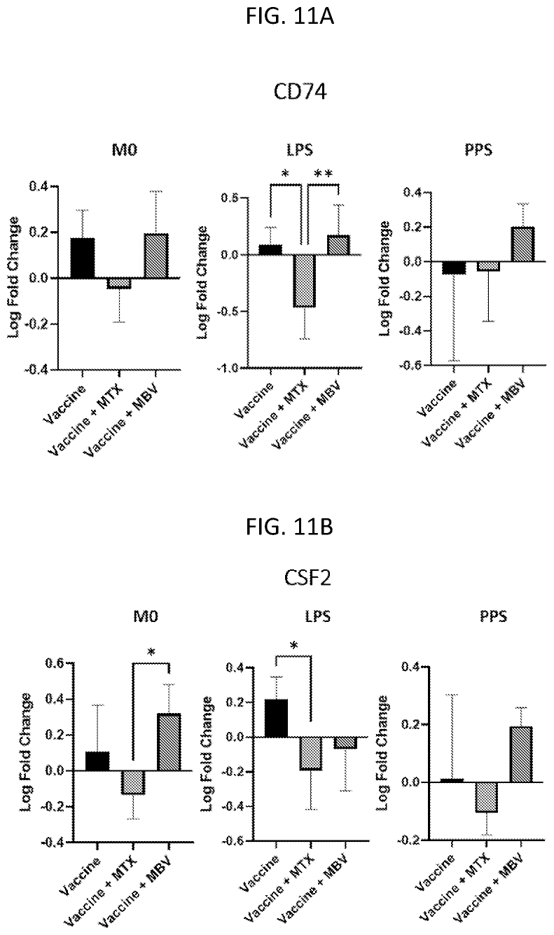

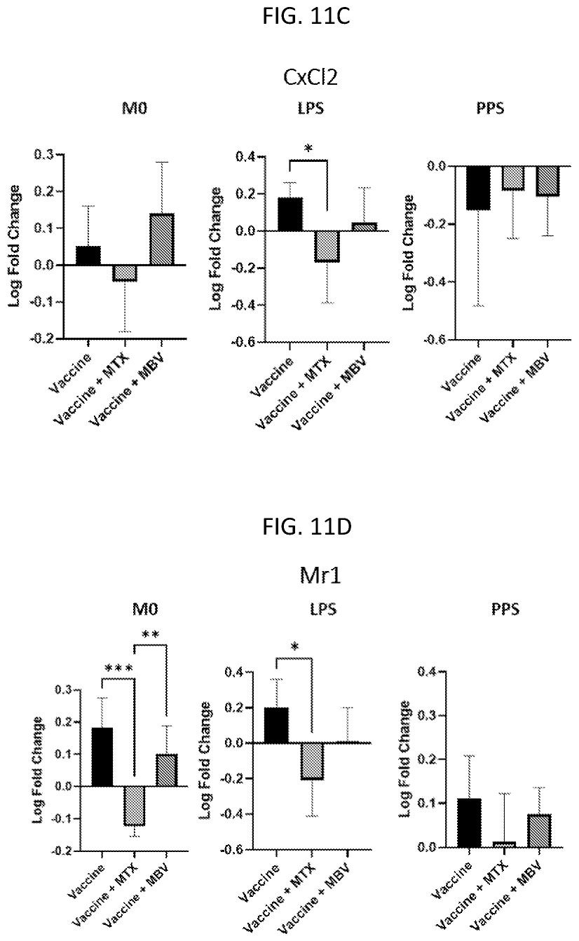

Relevant genes for antigen presentation (CD74 (MHCII); MR1 (MHC I)) and chemokines involved in immune cell recruitment (CSF2 and CXCL2) were not affected by repeated doses of MBV. This result confirmed that MBV do not compromise the humoral immune response (FIGS. 11A-11D).

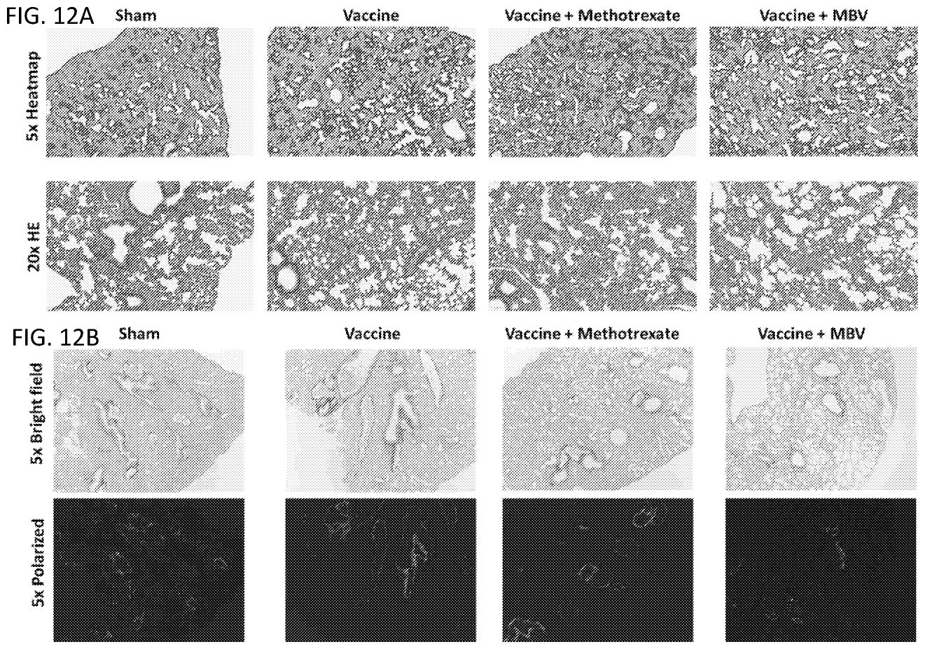

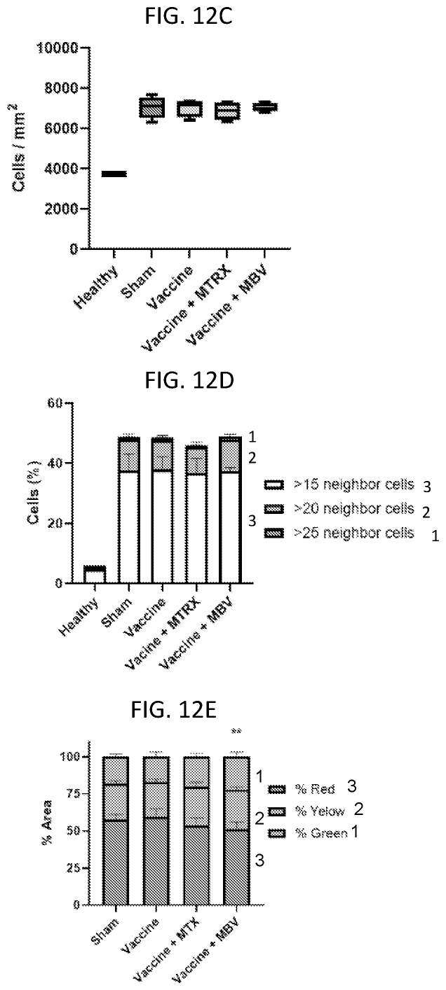

Histological analyses of lung tissue samples from the animals are shown in FIG. 12A-12E. The data showed no differences in cellularity among groups. However, mature collagen deposition was lower in vaccine+MBV groups, indicating lower levels of fibrosis caused by infection in the MBV treated animals compared to other groups (FIG. 12E). Reduction in fibrosis is associated with reduction in loss of lung function resulting from infection.

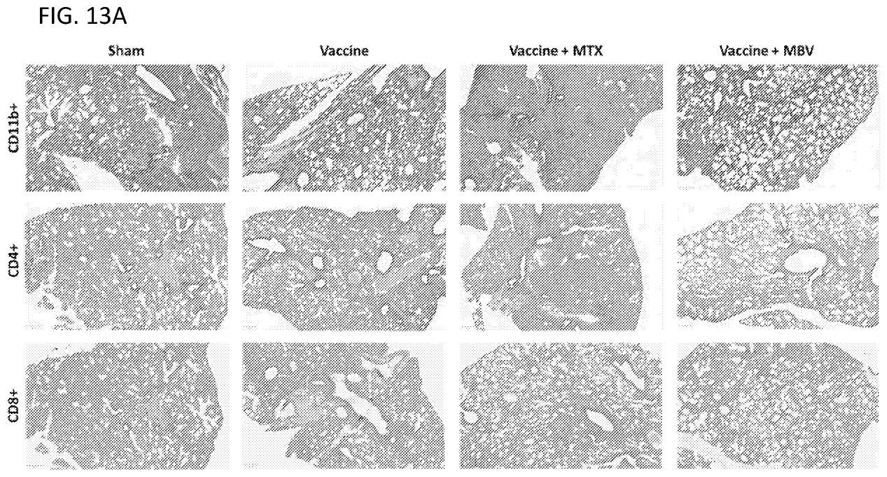

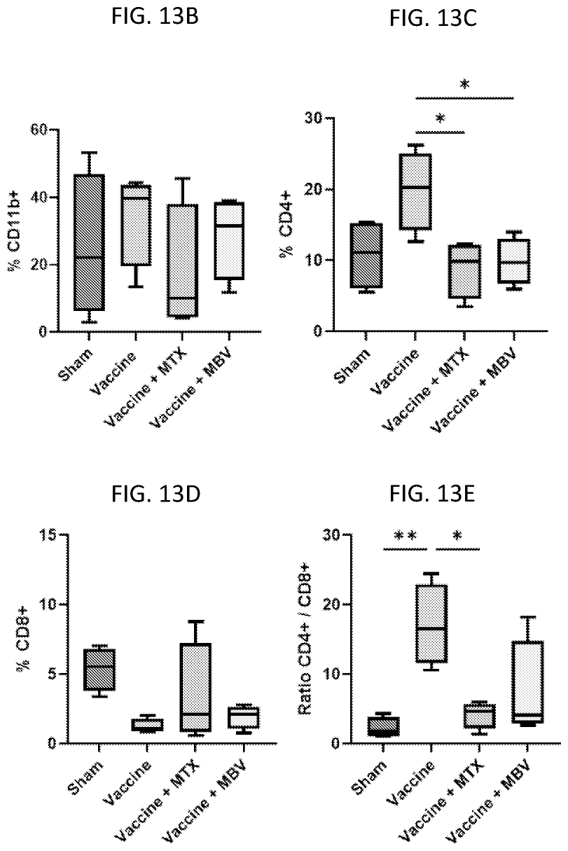

Immunolabelling of lung tissues, as shown in FIGS. 13A-13E, showed no differences in CD11b+ or CD8+ positive cells, but a higher presence of CD4+ cells in the vaccine only group. The level of CD4+ cells was lower in animals treated with vaccine+MBV, as compared to vaccine only, although the CD4+/CD8+ ratio was not significantly different between vaccine only and vaccine+MBV groups. These results suggest that MBV administration does not does not impact myeloid cell generation, and also suggests that MBV elicits a specific modulation of the immune response with immunosuppression.

This study demonstrated that MBV do not compromise the capacity of mice to produce a humoral response to a vaccine antigen. This capacity, measured by specific antibody levels, was maintained after an extended and repeated systemic administration of MBV both after first (vaccine immunization, as shown in FIGS. 5A-5B and FIGS. 6A-6B) and second (infection, as shown in FIGS. 8A-8B) exposure to antigens. Further, mice treated with both vaccine and MBV unexpectedly had a higher resistance to septic infection, as demonstrated by overall survival (FIG. 7).

MBV are able to modulate the phenotype of macrophages and affect the memory of these cells in response to different pathogen-derived antigens. Surprisingly, the current study demonstrated that macrophages from mice treated with MBV+vaccine had a broader cytokine response (such as higher IFNγ and IL23 production in response to LPS, as shown in FIGS. 9A-9D and FIG. 10) than macrophages from mice treated with vaccine only (no MBVs), while having a similar response to previously encountered (PPS). In addition, at the histopathological level, mice treated with MBV showed a modulation in fibrotic collagen deposition and CD4+ cell response, indicating that MBV modulate the immune response without immunosuppressive effects.

In view of the many possible aspects to which the principles of our invention may be applied, it should be recognized that illustrated aspects are only examples of the invention and should not be considered a limitation on the scope of the invention. Rather, the scope of the invention is defined by the following claims. We therefore claim as our invention all that comes within the scope and spirit of these claims.

1. A method of inducing an immune response to a vaccine antigen in a subject comprising:
administering to the subject an effective amount of isolated mammalian extracellular matrix bound vesicles (MBV), and an effective amount of a vaccine comprising or encoding the vaccine antigen,
wherein the MBV do not express CD63 and CD81 or are CD63^{lo}CD81^{lo},
thereby inducing the immune response to the vaccine antigen.

2. The method of claim 1, wherein the subject is a human.

3. The method of claim 1, wherein the immune response comprises (i) a therapeutic immune response; (ii) a protective immune response; (iii) induces myeloid cells; (iv) induces production of IgM and/or IgG antibodies to the vaccine antigen; and/or (v) induces production of IFNγ and/or IL-23.

4. (canceled)

5. (canceled)

6. (canceled)

7. (canceled)

8. The method of claim 1, wherein the vaccine antigen is a tumor associated antigen, a tumor cell or portion thereof, a viral antigen, a fungal antigen, a parasitic antigen, or a bacterial antigen.

9. The method of claim 1, wherein the vaccine comprises mRNA encoding the vaccine antigen.

10. (canceled)

11. The method of claim 1, wherein the vaccine comprises a live or inactivated attenuated virus, bacteria, fungus, parasite, or portion thereof.

12. (canceled)

13. (canceled)

14. (canceled)

15. (canceled)

16. The method of claim 1, wherein the vaccine induces an immune response against a virus, wherein the virus is an Avian herpesvirus, a Bovine herpesvirus, a Canine herpesvirus, an Equine herpesvirus, herpes simplex virus-1 (HSV-1), herpes simplex virus-2 (HSV-2), Feline viral rhinotracheitis virus, Marek's disease virus, an Ovine herpesviruses, a Porcine herpesvirus, Pseudorabies virus, an Avian paramyxovirus, Bovine respiratory syncytial virus, Human respiratory syncytial virus (RSV), Canine distemper virus, Canine parainfluenza virus, canine adenovirus, canine parvovirus, monkeypox virus, Bovine Parainfluenza virus 3, Ovine parainfluenza 3, human parainfluenza, Rinderpest virus, Border disease virus, Bovine viral diarrhea virus (BVDV), BVDV Type I, BVDV Type II, chikungunya virus, Classical swine fever virus, cytomegalovirus (CMV), Avian Leukosis virus, Bovine immunodeficiency virus, Bovine leukemia virus, Bovine tuberculosis, Ebola virus, Epstein Barr Virus (EBV), Equine infectious anemia virus, Feline immunodeficiency virus, Feline leukemia virus (FeLV), a coronavirus, monkeypox virus, Newcastle Disease virus, Ovine progressive pneumonia virus, Ovine pulmonary adenocarcinoma virus, Canine coronavirus (CCV), pantropic CCV, Canine respiratory coronavirus, Bovine coronavirus, Feline Calicivirus, Feline enteric coronavirus, Feline infectious peritonitis, a paramyxovirus, Porcine epidemic diarrhea virus, Porcine hemagglutinating encephalomyelitis virus, polio virus, Porcine parvovirus, Porcine Circovirus (PCV) Type I, PCV Type II, Porcine Reproductive and Respiratory Syndrome (PRRS) Virus, hepatitis virus, Rubella virus, Transmissible gastroenteritis virus, Turkey coronavirus, Bovine ephemeral fever virus, Rabies virus, small pox (variola) virus, Rotavirus, variola virus, varicella zoster virus, Vesicular stomatitis virus, lentivirus, Avian influenza, Rhinoviruses, Equine influenza virus, Swine influenza virus, Canine influenza virus, Feline influenza virus, Human influenza virus, Eastern Equine encephalitis virus (EEE), Venezuelan equine encephalitis virus, West Nile virus, Western equine encephalitis virus, human immunodeficiency virus, human papilloma virus, varicella zoster virus, hepatitis B virus, rhinovirus, and measles virus, severe acute respiratory syndrome coronavirus 1 (SARS-COV-1), severe acute respiratory syndrome coronavirus 2 (SARS-COV-2), a coronavirus, zika virus, a paramyxovirus, polio virus, hepatitis A virus, hepatitis C virus, Rubella virus, human parvovirus, norovirus, mumps virus, molluscum contagiosum virus, Rubeola virus, enterovirus, coxsackievirus, a picornavirus, or a herpesvirus.

17. The method of claim 1, wherein the vaccine induces an immune response against a bacteria, wherein the bacteria is Acinetobacter baumanii, an Actinobacillus sp., Actinomycetes, an Actinomyces sp. an Aeromonas sp., Anaplasma phagocytophilum, Alcaligenes xylosoxidans, Acinetobacter baumanii, Actinobacillus actinomycetemcomitans, a Bacillus sp., a Bacteroides sp., a Bartonella sp., Bifidobacterium sp., a Bordetella sp., a Borrelia sp., a Brucella sp., a Burkholderia sp., a Campylobacter sp., Capnocytophaga sp., Cardiobacterium hominis, Chlamydia trachomatis, Chlamydophila pneumoniae, Chlamydophila psittaci, a Citrobacter sp. Coxiella burnetii, Corynebacterium sp., a Clostridium sp., Eikenella corrodens, an Enterobacter sp. Escherichia coli, an Enterococcus sp., an Ehrlichia sp., Erysipelothrix rhusiopathiae, an Eubacterium sp., Francisella tularensis, Fusobacterium nucleatum, Gardnerella vaginalis, Gemella morbillorum, a Haemophilus sp, Helicobacter sp., Kingella kingii, a Klebsiella sp., a Lactobacillus sp., Listeria monocytogenes, Leptospira interrogans, Legionella pneumophila, Leptospira interrogans, a Peptostreptococcus sp., Moraxella catarrhalis, a Morganella sp., a Mobiluncus sp., a Micrococcus sp., a Mycobacterium sp., a Mycoplasma sp., a Nocardia sp., a Neisseria sp., Pasteurella multocida, Plesiomonas shigelloides, a Prevotella sp., a Porphyromonas sp., Prevotella melaninogenica, a Proteus sp., a Providencia sp., Pseudomonas aeruginosa, Propionibacterium acnes, Rhodococcus equi, a Rickettsia sp., a Rhodococcus sp., Serratia marcescens, Stenotrophomonas maltophilia, Salmonella sp., a Serratia sp., a Shigella sp., a Staphylococcus sp., a Streptococcus sp., Spirillum minus, Streptobacillus moniliformi, a Treponema sp., Tropheryma whippelii, Ureaplasma urealyticum, a Veillonella sp., a Vibrio sp., a Yersinia sp. and Xanthomonas maltophilia.

18. The method of claim 17, wherein the bacteria is a Streptococcus sp. or S. pneumoniae.

19. (canceled)

20. (canceled)

21. (canceled)

22. The method of claim 21, wherein the antigen is lipoteichoic acid (LTA) or lipopolysaccharide (LPS).

23. (canceled)

24. (canceled)

25. (canceled)

26. The method of claim 1, wherein the vaccine induces an immune response against a fungus, wherein the fungus is Trichophyton rubrum, T. mentagrophytes, Epidermophyton floccosum, Microsporum canis, Pityrosporum orbiculare, a Candida sp., an Aspergillus sp.), a Cryptococcus sp., a Histoplasma sp., a Pneumocystis sp., or a Stachybotrys.

27. The method of claim 1, wherein the vaccine induces an immune response against a parasite, wherein the parasite is a Plasmodium, a Schistosome, a Trypanosome, a filarial nematodes, trichomoniasis, sarcosporidiasis, Taenia, Leishmania, Toxoplasma gondii, Trichinelosis or Coccidiosis.

28. The method of claim 1, wherein the vaccine induces a therapeutic immune response against a tumor cell in the subject, and wherein the therapeutic response is a reduction in tumor volume, tumor metastasis, or tumor number.

29. The method of claim 1, wherein if the subject is infected with a pathogen to which the vaccine induces a protective immune response, the subject experiences increased IFNγ and/or IL-23 production compared to if the subject had received the vaccine without MBV.

30. (canceled)

31. The method of claim 1, wherein the matrix bound vesicles
(a) contain miR-145 and miR-181;
(b) do not comprise alkaline phosphatase; and/or
(c) do not comprise or have barely detectable levels of EpCAM, ANXA5, TSG101, GM130, FLOT1, ICAM1, and/or ALIX1.

32. The method of claim 1, wherein the matrix bound vesicles comprise:
(a) a phospholipid content comprising at least 55% phosphatidylcholine (PC) and phosphatidyl inositol (PI) in combination;
(b) a phospholipid content comprising 10% or less sphingomyelin (SM);
(c) a phospholipid content comprising 20% or less phosphatidylethanolamine (PE); and/or
(d) a phospholipid content comprising 15% or greater phosphatidylinositol (PI).

33. (canceled)

34. The method of claim 1, wherein the vaccine and the MBV are each administered separately to the subject.

35. (canceled)

36. The method of claim 1, wherein the vaccine and the MBV are administered to the subject on a first date and the subject subsequently receives, on one or more subsequent dates, a further administration of an effective amount of MBV without further administration of vaccine.

37. (canceled)

38. (canceled)

39. The method of claim 1, wherein the vaccine and/or the MBV are administered by intramuscular injection.

40.

41. The method of claim 1, wherein the MBV are administered in an amount selected from 1×10⁶ to 1×10²⁰, 1×10⁶ to 1×10¹², or 1×10⁹ to 1×10¹⁴ MBV per kg of body weight per administration.

42. (canceled)

43. (canceled)

44. The method of claim 1, wherein the MBV are derived from extracellular matrix of urinary bladder, small intestine, heart, dermis, liver, kidney, uterus, brain, blood vessel, lung, bone, muscle, pancreas, placenta, stomach, spleen, colon, adipose tissue, or esophagus.

45. The method of claim 1, wherein the MBV are derived from urinary bladder matrix (UBM), small intestinal submucosa (SIS), or urinary bladder submucosa (UBS)

46. The method of claim 1, wherein the mammal is a pig, cow, or sheep.

47. The method of claim 1, further comprising administering an effective amount of an adjuvant, wherein the adjuvant is interleukin (IL)-12.

48. The method of claim 1, wherein the method induces an immune response to an antigen that is not the vaccine antigen and/or induces production of IFNγ and/or IL-23.

49. (canceled)

50. (canceled)

51. (canceled)

52. (canceled)

53. (canceled)

54. A pharmaceutical composition comprising an effective amount of isolated mammalian extracellular matrix bound vesicles (MBV) that do not express CD63 and CD81 or are CD63^{lo}CD81^{lo}, an effective amount of a vaccine comprising or encoding a vaccine antigen, and a pharmaceutically acceptable carrier.

55-75. (canceled)


SEQUENCE LISTING (7 sequences)

SEQ ID NO: 1 - RNA, 88 nt, Homo sapiens
  source 1..88: mol_type = other RNA
caccttgtcctcacggtccagttttcccaggaatcccttagatgctaagatggggattcctggaaatactgttcttgaggtcatggtt

SEQ ID NO: 2 - RNA, 110 nt, Homo sapiens
  source 1..110: mol_type = other RNA
agaagggctatcaggccagccttcagaggactccaaggaacattcaacgctgtcggtgagtttgggatttgaaaaaaccactgaccgttgactgtaccttggggtcctta

SEQ ID NO: 3 - RNA, 106 nt, Homo sapiens
  source 1..106: mol_type = other RNA
gcgcagcgccctgtctcccagcctgaggtgcagtgctgcatctctggtcagttgggagtctgagatgaagcactgtagctcaggaagagagaagttgttctgcagc

SEQ ID NO: 4 - RNA, 129 nt, synthetic construct
  source 1..129: mol_type = other RNA
tctcaacacaacatatacaaaacaaacgaatctcaagcaatcaagcattctacttctattgcagcaatttaaatcatttcttttaaagcaaaagcaattttctgaaaattttcaccatttacgaacgat

SEQ ID NO: 5 - RNA, 49 nt, synthetic construct
  source 1..49: mol_type = other RNA
cgaactagtattcttctggtccccacagactcagagagaacccgccacc

SEQ ID NO: 6 - RNA, 15 nt, synthetic construct
  source 1..15: mol_type = other RNA
aggagggtttttacc

SEQ ID NO: 7 - RNA, 122 nt, synthetic construct
  source 1..122: mol_type = other RNA
aaccagcctcaagaacacccgaatggagtctctaagctacataataccaacttacacttacaaaatgttgtcccccaaaatgtagccattcgtatctgctcctaataaaaagaaagtttctt